(19) United States
(12) Patent Application Publication
(10) Pub. No.: US 2024/0237529 A1
(43) Pub. Date: Jul. 11, 2024

(54) NOVEL COMPOUND, AND PHOTOELECTRIC DEVICE, IMAGE SENSOR, AND ELECTRONIC DEVICE INCLUDING SAME

(71) Applicants: Samsung Electronics Co., Ltd., Suwon-si (KR); Sogang University Research & Business Development Foundation, Seoul (KR)

(72) Inventors: Sangdong KIM, Suwon-si (KR); Ohkyu KWON, Suwon-si (KR); Rae Sung KIM, Suwon-si (KR); Sang Ho PARK, Suwon-si (KR); Insun PARK, Suwon-si (KR); Duck-hyung LEE, Seoul (KR); Dong-Seok LEEM, Suwon-si (KR); Chan-ju JEONG, Seoul (KR); Eun-gyeong CHOI, Daegu (KR); Mi-hyeon CHOO, Seoul (KR)

(73) Assignees: Samsung Electronics Co., Ltd., Suwon-si (KR); Sogang University Research & Business Development Foundation, Seoul (KR)

(21) Appl. No.: 18/515,647

(22) Filed: Nov. 21, 2023

(30) Foreign Application Priority Data
Nov. 21, 2022 (KR) 10-2022-0156802

(51) Int. Cl. H10K 85/60; C07C 25/22; C07D 339/06; H10K 30/30; H10K 30/40; H10K 39/32

(52) U.S. Cl. CPC H10K 85/6576; C07C 25/22; C07D 339/06; H10K 85/615; H10K 85/623; H10K 85/624; C07C 2603/52; C07C 2603/54; H10K 30/30; H10K 30/40; H10K 39/32

(57) ABSTRACT

Provided are a compound represented by Chemical Formula 1, a photoelectric device, an image sensor, and an electronic device including the same.

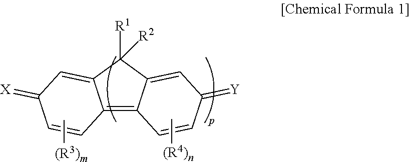

In Chemical Formula 1, the definition of each substituent is as described in the specification.

### CROSS-REFERENCE TO RELATED APPLICATION

This application claims priority to and the benefit of Korean Patent Application No. 10-2022-0156802 filed in the Korean Intellectual Property Office on Nov. 21, 2022, the entire contents of which are incorporated herein by reference.

### BACKGROUND

### 1. Field

A novel compound, and a photoelectric device, an image sensor, and an electronic device including the compound are disclosed.

### 2. Description of the Related Art

A photoelectric device may convert light into an electrical signal using photoelectric effects. A photoelectric device may include a photodiode, a phototransistor, etc., and may be applied to an image sensor, etc.

An image sensor including a photodiode requires high resolution and thus a small pixel. At present, a silicon photodiode is widely used. In some cases, a silicon photodiode exhibits a problem of deteriorated sensitivity because of a relatively small absorption area due to relatively small pixels. Accordingly, an organic material that is capable of replacing silicon has been researched.

### SUMMARY

Some example embodiments provide a novel compound. The novel compound may include an organic material that may have a relatively high extinction coefficient and may selectively absorb light in a particular wavelength region depending on a molecular structure, and thus may simultaneously replace a photodiode and a color filter and resultantly improve sensitivity and contribute to relatively high integration.

Some example embodiments also provide a near-infrared absorbing and/or blocking film including the novel compound.

Some example embodiments also provide an photoelectronic device including the novel compound.

Some example embodiments also provide an image sensor including the photoelectric device.

Some example embodiments also provide an organic sensor including the image sensor.

Some example embodiments also provide an electronic device including the organic sensor.

The compound according to some example embodiments may be represented by Chemical Formula 1:

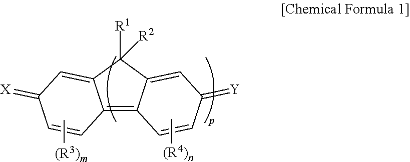

In Chemical Formula 1,
R¹ and R² may each independently be hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³ and R⁴ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
m and n may each independently be one of integers of 0 to 3,
p may be one of integers of 1 to 5, and
X and Y may each independently be an electron-acceptor moiety, an electron-donor moiety, or any combination thereof,
when p is 1 and X and Y are the same electron-acceptor moiety, R¹ and R² are not simultaneously a substituted or unsubstituted C1 to C30 saturated aliphatic hydrocarbon group (e.g., are not both substituted or unsubstituted C1 to C30 saturated aliphatic hydrocarbon groups),
when p is 1 and one of X or Y is an electron-acceptor moiety and another one of X or Y is an electron-donor moiety, both R¹ and R² are not hydrogens at the same time, and/or are not methyl groups at the same time (e.g., are not both hydrogens and are not both methyl groups), and
when p is 2, X and Y do not include a same electron-acceptor moiety.

The electron-acceptor moiety may be represented by (CN)₂—C═*, wherein * is a portion linked to a position of X in Chemical Formula 1 or a position of Y in Chemical Formula.

The electron-donor moiety may be represented by Chemical Formula 2:

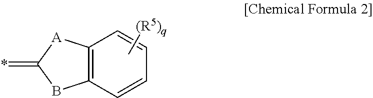

wherein, in Chemical Formula 2,
A and B may each independently be oxygen (O), sulfur (S), selenium (Se), or tellurium (Te),
R⁵ may be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q may be one of integers of 0 to 4, and
* may be a portion linked to a position of X in Chemical Formula 1 or a position of Y in Chemical Formula 1.

In Chemical Formula 1,
R¹ to R⁴ may each independently be deuterium, a halogen, a cyano group, a nitro group, a substituted or unsubstituted C1 to C10 alkyl group, a substituted or unsubstituted C6 to C20 aryl group, or any combination thereof,
m and n are each independently an integer of 0 or 1, and
p is one of integers of 1 to 4.

In Chemical Formula 1,
when p is 1 and one of X or Y is an electron-acceptor moiety and another one of X or Y is an electron-donor moiety, R¹ and R² may each independently be a halogen, a cyano group, a nitro group, a substituted or unsubstituted C2 to C20 alkyl group, a substituted or unsubstituted C6 to C20 aryl group, or any combination thereof.

In Chemical Formula 1,
when p is 1 and X and Y are each a same electron-donor moiety, R¹ and R² may each independently be hydrogen, a halogen, a cyano group, a nitro group, a substituted or unsubstituted C1 to C20 alkyl group, a substituted or unsubstituted C6 to C20 aryl group, or any combination thereof, provided that R¹ and R² are not simultaneously a substituted or unsubstituted C1 to C20 alkyl group.

In Chemical Formula 1,
when p is 2, X may be represented by (CN)₂—C═* (wherein * is a portion linked to a position of X in Chemical Formula 1), and Y may be represented by Chemical Formula 2.

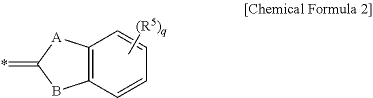

In Chemical Formula 2,
A and B are each independently oxygen (O), sulfur (S), selenium (Se), or tellurium (Te),
R⁵ is deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to a position of Y in Chemical Formula 1.

In Chemical Formula 1,
when p is 2, both X and Y may be represented by Chemical Formula 2.

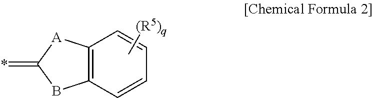

In Chemical Formula 2,
A and B are each independently oxygen (O), sulfur (S), selenium (Se), or tellurium (Te),
R⁵ is deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to a position of X in Chemical Formula 1 or a position of Y in Chemical Formula 1.

In Chemical Formula 1,
R¹ and R² are each independently a halogen, a substituted or unsubstituted C1 to C5 alkyl group, or any combination thereof,
m and n are each independently 0 or 1,
p is one of integers of 1 to 4,

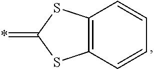 [SMILES: *=C1Sc2ccccc2S1]

X and Y may each independently be (CN)₂—C═*, s, or any combination thereof, wherein * is a portion linked to the X or Y position of Chemical Formula 1,
when p is 1, at least one of R¹ or R² may be a halogen, and
when p is 2, X and Y may be different from each other.

Chemical Formula 1 is represented by one or more of Chemical Formula 1-1 to Chemical Formula 1-3:

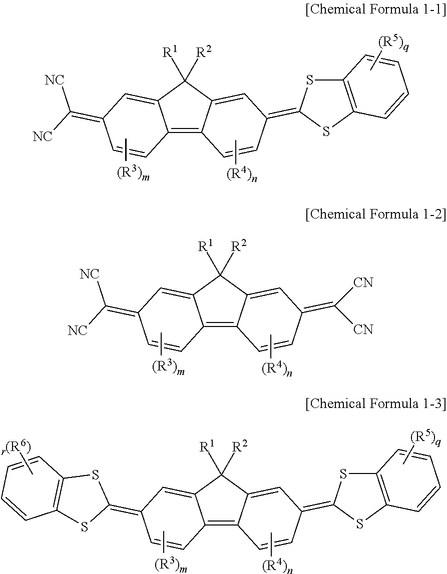

wherein, in Chemical Formulas 1-1 to 1-3,
R¹ and R² may each independently be hydrogen, deuterium, a halogen, or a substituted or unsubstituted C1 to C30 alkyl group,
R³ and R⁴ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
m and n may each independently be one of integers of 0 to 3,
R⁵ and R⁶ may each independently be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q and r may each independently be one of integers from 0 to 4,
in Chemical Formula 1-1, R¹ and R² may not be simultaneously hydrogen or a methyl group, and
in Chemical Formula 1-2, R¹ and R² may not be simultaneously a substituted or unsubstituted C1 to C30 alkyl group.

Chemical Formula 1 may be represented by Chemical Formula 1-4 or Chemical Formula 1-5:

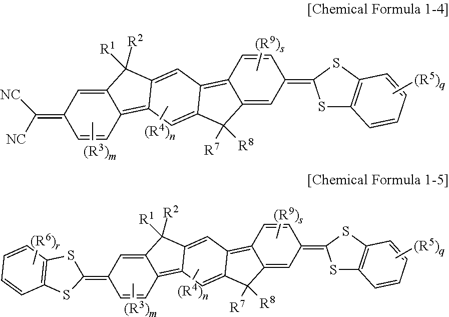

wherein, in Chemical Formula 1-4 and Chemical Formula 1-5,
R¹, R², R¹, and R^{a} may each independently be hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³, R⁴, and R⁹ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R⁵ and R⁶ may each independently be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
m and s may each independently be one of integers of 0 to 3,
n is one of integers of 0 to 2, and
q and r may each independently be one of integers of 0 to 4.

Chemical Formula 1 may be represented by Chemical Formula 1-6 or Chemical Formula 1-7:

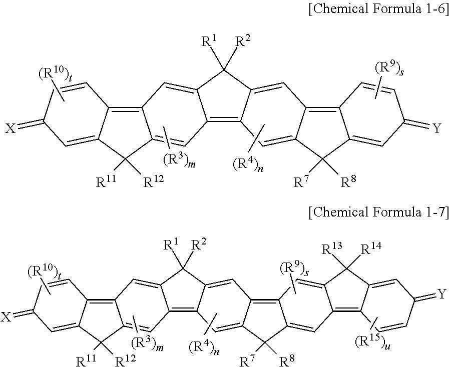

wherein, in Chemical Formula 1-6 and Chemical Formula 1-7,
R¹, R², R⁷, R⁸, R¹¹, R¹², R¹³, and R¹⁴ may each independently be hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³, R⁴, R⁹, R¹⁰, and R¹⁵ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
X and Y may each independently be an electron-acceptor moiety, an electron-donor moiety, or any combination thereof,
in Chemical Formula 1-6, m and n may each independently be one of integers of 0 to 2, and s and t may each independently be one of integers of 0 to 3, and

In Chemical Formula 1-7, m, n, and s may each independently be one of integers of 0 to 2, and t and u may each independently be one of integers of 0 to 3.

In Chemical Formula 1-6 and Chemical Formula 1-7, the electron-acceptor moiety is represented by (CN)₂—C═*, wherein * is a portion linked to a position of X in Chemical Formula 1-6 or Chemical Formula 1-7 or a position of Y in Chemical Formula 1-6 or Chemical Formula 1-7, and the electron-donor moiety is represented by Chemical Formula 2:

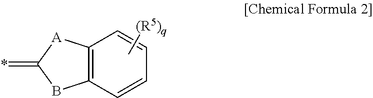

wherein, in Chemical Formula 2,
A and B may each independently be oxygen (O), sulfur (S), selenium (Se), or tellurium (Te),
R⁵ may be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to the position of X in Chemical Formula 1-6 or Chemical Formula 1-7 or the position of Y in Chemical Formula 1-6 or Chemical Formula 1-7.

A molecular weight of the compound may range from about 250 g/mol to about 1,000 g/mol.

A peak absorption wavelength of the compound may fall within the wavelength range of about 750 nm to about 3,000 nm.

A near-infrared absorbing and/or blocking film according to some example embodiments includes the aforementioned compound.

A photoelectric device according to some example embodiments includes a first electrode and a second electrode facing each other, and an active layer between the first electrode and the second electrode, wherein the active layer includes the compound represented by Chemical Formula 1.

A peak absorption wavelength of the active layer ranges from about 750 nm to about 3,000 nm.

A photoelectric device according to some example embodiments includes a first electrode and a second electrode facing each other, an active layer between the first electrode and the second electrode, and at least one charge auxiliary layer between the active layer and the first electrode and/or between the active layer and the second electrode, and the charge auxiliary layer includes the compound represented by Chemical Formula 1.

An image sensor according to some example embodiments includes a photoelectric device according to some example embodiments.

An electronic device according to some example embodiments includes the photoelectric device.

An electronic device according to some example embodiments includes the image sensor.

The compound according to some example embodiments exhibits good light absorption characteristics in the near-infrared region, and for example, it exhibits excellent light absorption characteristics in the near-infrared wavelength of a longer wavelength region. Therefore, the compound according to some example embodiments can be used as a near-infrared absorber, and can be advantageously applied to near-infrared absorbing and/or blocking films, photoelectric devices, image sensors, electronic devices, and the like, including the compound.

### BRIEF DESCRIPTION OF THE DRAWINGS

FIG. 1 is a cross-sectional view showing a photoelectric device according to some example embodiments,

FIG. 2 is a cross-sectional view showing a photoelectric device according to some example embodiments,

FIG. 3 is a cross-sectional view showing an organic sensor according to some example embodiments,

FIG. 4 is a cross-sectional view showing an organic sensor according to some example embodiments,

FIG. 5 is a cross-sectional view showing an organic sensor according to some example embodiments,

FIG. 6 is a schematic view showing an example of a pixel array of an organic sensor according to some example embodiments,

FIG. 7 is a cross-sectional view showing an organic sensor according to some example embodiments,

FIG. 8 is a cross-sectional view showing an organic sensor according to some example embodiments,

FIG. 9 is a cross-sectional view showing an organic sensor according to some example embodiments,

FIG. 10 is a cross-sectional view showing an organic sensor according to some example embodiments,

FIG. 11 is a cross-sectional view showing an organic sensor according to some example embodiments,

FIG. 12 is a block diagram of a digital camera including an organic sensor according to some example embodiments,

FIG. 13 is a schematic view of an electronic device according to some example embodiments,

FIG. 14 shows absorption spectra of Compound 1, Compound 2, and Compound a prepared in Synthesis Example 1, Synthesis Example 2, and Comparative Synthesis Example 1, respectively, in a solution state.

### DETAILED DESCRIPTION

Hereinafter, some example embodiments will hereinafter be described in detail, and may be easily performed by a person having an ordinary skill in the related art. However, the inventive concepts may be embodied in many different forms and are not to be construed as limited to the example embodiments set forth herein.

In the drawings, the thickness of layers, films, panels, regions, etc., are exaggerated for clarity. In the drawings, parts having no relationship with the description are omitted for clarity of the embodiments, and the same or similar constituent elements are indicated by the same reference numeral throughout the specification.

It will be understood that when an element such as a layer, film, region, or substrate is referred to as being “on” another element, it may be directly on the other element or intervening elements may also be present. In contrast, when an element is referred to as being “directly on” another element, there are no intervening elements present. When a component is referred to as being “on” another component, it means that it may be in contact with the upper or lower portion of the other component.

As used herein, when specific definition is not otherwise provided, “substituted” refers to replacement of a hydrogen of a compound by a substituent of a halogen atom (F, Br, Cl, or I), a hydroxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a C1 to C20 alkyl group, a C1 to C20 alkoxy group, a C2 to C20 alkenyl group, a C2 to C20 alkynyl group, a C6 to C30 aryl group, a C7 to C30 arylalkyl group, a C2 to C20 heteroaryl group, C3 to C20 heteroarylalkyl group, a C3 to C30 cycloalkyl group, a C3 to C15 cycloalkenyl group, a C6 to C15 cycloalkynyl group, a C2 to C20 heterocycloalkyl group, or any combination thereof.

As used herein, when specific definition is not otherwise provided, “hetero” refers to one including 1 to 3 heteroatoms of N, O, S, P, or S.

As used herein, “alkyl group” includes, for example, a methyl group, an ethyl group, a propyl group, an isopropyl group, an n-butyl group, an isobutyl group, a t-butyl group, a pentyl group, a hexyl group, and the like.

As used herein, “cycloalkyl group” refers to a cyclopropyl group, a cyclobutyl group, a cyclopentyl group, a cyclohexyl group, and the like.

As used herein, “aryl group” refers to a substituent including all element of the functional group having p-orbitals which form conjugation, and may be a monocyclic, polycyclic or fused-ring polycyclic (e.g., rings sharing adjacent pairs of carbon atoms) functional group.

As used herein, when a definition is not otherwise provided, “cyano-containing group” refers to a monovalent group such as a C1 to C30 alkyl group, a C2 to C30 alkenyl group, or a C2 to C30 alkynyl group where at least one hydrogen is substituted with a cyano group. As used herein, when a definition is not otherwise provided, the cyano-containing group also refers to a divalent functional group such as a dicyanoalkenyl group represented by ═CR^{x′}—(CR^{x}R^{y})ₚ—CR^{y′}(CN)₂ wherein R^{x}, R^{y}, R^{x′}, and R^{y′} are the same or different and are each independently hydrogen or a C1 to C10 alkyl group and p is an integer of 0 to 10 (or 1 to 10). Examples of the cyano-containing group may be a dicyanomethyl group, a dicyanovinyl group, a cyanoethynyl group, and the like.

Unless otherwise defined herein, “heterocycle” refers to a ring including at least one hetero atom, wherein the hetero atom may be selected from N, O, S, P and Si, and the ring is an aliphatic ring, aromatic rings, or fused rings thereof.

As used herein, when a definition is not otherwise provided, “combination thereof” refers to at least two substituents bound to each other by a single bond or a C1 to C10 alkylene group, or at least two fused substituents.

As used herein, “5-membered aromatic ring” refers to a 5-membered cyclic group (e.g., C5 aryl group) providing a conjugated structure or a 5-membered heterocyclic group (e.g., C2 to C4 heteroaryl group) providing a conjugated structure) but is not limited thereto. As used herein, “6-membered aromatic ring” refers to a 6-membered cyclic group (e.g., a C6 aryl group) providing a conjugated structure or a 6-membered heterocyclic group (e.g., a C2 to C5 heteroaryl group) providing a conjugated structure), but is not limited thereto. In addition, the aromatic ring may include the 5-membered aromatic ring or the 6-membered aromatic ring, but is not limited thereto.

It will further be understood that when an element is referred to as being “on” another element, it may be above or beneath or adjacent (e.g., horizontally adjacent) to the other element. It will be understood that elements and/or properties thereof (e.g., structures, surfaces, directions, or the like), which may be referred to as being “perpendicular,” “parallel,” “coplanar,” or the like with regard to other elements and/or properties thereof (e.g., structures, surfaces, directions, or the like) may be “perpendicular,” “parallel,” “coplanar,” or the like or may be “substantially perpendicular,” “substantially parallel,” “substantially coplanar,” respectively, with regard to the other elements and/or properties thereof. Elements and/or properties thereof (e.g., structures, surfaces, directions, or the like) that are “substantially perpendicular” with regard to other elements and/or properties thereof will be understood to be “perpendicular” with regard to the other elements and/or properties thereof within manufacturing tolerances and/or material tolerances and/or have a deviation in magnitude and/or angle from “perpendicular,” or the like with regard to the other elements and/or properties thereof that is equal to or less than 10% (e.g., a tolerance of ±10%). Elements and/or properties thereof (e.g., structures, surfaces, directions, or the like) that are “substantially parallel” with regard to other elements and/or properties thereof will be understood to be “parallel” with regard to the other elements and/or properties thereof within manufacturing tolerances and/or material tolerances and/or have a deviation in magnitude and/or angle from “parallel,” or the like with regard to the other elements and/or properties thereof that is equal to or less than 10% (e.g., a tolerance of ±10%). Elements and/or properties thereof (e.g., structures, surfaces, directions, or the like) that are “substantially coplanar” with regard to other elements and/or properties thereof will be understood to be “coplanar” with regard to the other elements and/or properties thereof within manufacturing tolerances and/or material tolerances and/or have a deviation in magnitude and/or angle from “coplanar,” or the like with regard to the other elements and/or properties thereof that is equal to or less than 10% (e.g., a tolerance of ±10%). It will be understood that elements and/or properties thereof may be recited herein as being “identical” to, “the same” or “equal” as other elements, and it will be further understood that elements and/or properties thereof recited herein as being “identical” to, “the same” as, or “equal” to other elements may be “identical” to, “the same” as, or “equal” to or “substantially identical” to, “substantially the same” as or “substantially equal” to the other elements and/or properties thereof. Elements and/or properties thereof that are “substantially identical” to, “substantially the same” as or “substantially equal” to other elements and/or properties thereof will be understood to include elements and/or properties thereof that are identical to, the same as, or equal to the other elements and/or properties thereof within manufacturing tolerances and/or material tolerances. Elements and/or properties thereof that are identical or substantially identical to and/or the same or substantially the same as other elements and/or properties thereof may be structurally the same or substantially the same, functionally the same or substantially the same, and/or compositionally the same or substantially the same. It will be understood that elements and/or properties thereof described herein as being “substantially” the same and/or identical encompasses elements and/or properties thereof that have a relative difference in magnitude that is equal to or less than 10%. Further, regardless of whether elements and/or properties thereof are modified as “substantially,” it will be understood that these elements and/or properties thereof should be construed as including a manufacturing or operational tolerance (e.g., ±10%) around the stated elements and/or properties thereof. While the term “same,” “equal” or “identical” may be used in description of some example embodiments, it should be understood that some imprecisions may exist. Thus, when one element is referred to as being the same as another element, it should be understood that an element or a value is the same as another element within a desired manufacturing or operational tolerance range (e.g., ±10%). When the terms “about” or “substantially” are used in this specification in connection with a numerical value, it is intended that the associated numerical value includes a manufacturing or operational tolerance (e.g., ±10%) around the stated numerical value. Moreover, when the words “about” and “substantially” are used in connection with geometric shapes, it is intended that precision of the geometric shape is not required but that latitude for the shape is within the scope of the inventive concepts. Further, regardless of whether numerical values or shapes are modified as “about” or “substantially,” it will be understood that these values and shapes should be construed as including a manufacturing or operational tolerance (e.g., ±10%) around the stated numerical values or shapes. When ranges are specified, the range includes all values therebetween such as increments of 0.1%.

Hereinafter, a compound according to some example embodiments (also referred to herein interchangeably as a novel compound) is described.

The compound according to some example embodiments may be represented by Chemical Formula 1:

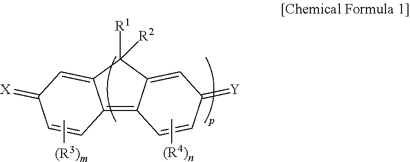

In Chemical Formula 1,
R¹ and R² may each independently be hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³ and R⁴ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
m and n may each independently be one of integers of 0 to 3,
p may be one of integers of 1 to 5, and
X and Y may each independently be an electron-acceptor moiety, an electron-donor moiety, or any combination thereof,
when p is 1 and X and Y are the same electron-acceptor moiety, R¹ and R² are not simultaneously substituted or unsubstituted C1 to C30 saturated aliphatic hydrocarbon groups (e.g., are not simultaneously a substituted or unsubstituted C1 to C30 saturated aliphatic hydrocarbon group, are not both substituted or unsubstituted C1 to C30 saturated aliphatic hydrocarbon groups, are not both a substituted or unsubstituted C1 to C30 saturated aliphatic hydrocarbon group, are not each a substituted or unsubstituted C1 to C30 saturated aliphatic hydrocarbon group, etc.),
when p is 1 and one of X or Y is an electron-acceptor moiety and another one of X or Y is an electron-donor moiety, both R¹ and R² are not hydrogens at the same time, and/or are not methyl groups at the same time (e.g., are not both hydrogens and are not both methyl groups, are not simultaneously a hydrogen or a methyl group, are not both a hydrogen and are not both a methyl group, are not each a hydrogen and are not each a methyl group, etc.), and
when p is 2, X and Y do not include the same electron-acceptor moiety (e.g., X and Y do not include a same electron-acceptor moiety).

The compound represented by Chemical Formula 1 according to some example embodiments is a quinoid-type compound based on a fluorene structure, and the number of repeating units indicated by ‘p’ in Chemical Formula 1 may be changed to adjust a conjugation length and a maximum absorption wavelength of the compound represented by Chemical Formula 1. In addition, the compound represented by Chemical Formula 1 includes an electron-acceptor moiety or an electron-donor moiety at X and Y present at both (e.g., opposite) terminal ends thereof, respectively, thereby further enhancing charge transfer characteristics of the compound. The compound represented by Chemical Formula 1 has a low bandgap energy and can effectively absorb light in the near-infrared wavelength region, and can be advantageously applied to a deposition process by adjusting the molecular weight of the compound to less than or equal to about 1,000 gram/mole.

Materials that absorb long-wavelength light, such as near-infrared rays, should have a small HOMO-LUMO bandgap energy. In order to have a small bandgap energy, a conjugate length may be long. However, if the conjugate length gets longer, it may be disadvantageous for application to the deposition process, and aggregation of compounds may occur during deposition. The compound represented by Chemical Formula 1 can easily control the conjugate length by adjusting the number of repeating units, and thus can easily control the achievement of a small bandgap energy that is advantageous for absorption of the near-declination wavelength. The addition of this conjugated structure may be appropriately adjusted within the range available for deposition. For example, the molecular weight may be adjusted to a range of less than or equal to about 1,000 g/mol, so that it can be advantageously applied to deposition. In addition, the compound of Chemical Formula 1 according to some example embodiments is expected to absorb long wavelengths of greater than or equal to about 800 nm, greater than or equal to about 900 nm, greater than or equal to about 1,000 nm, greater than or equal to about 1,100 nm, greater than or equal to about 1,200 nm, or greater than or equal to about 1,300 nm in a single molecule state without aggregation phenomenon.

The compound according to some example embodiments has a specific structure represented by Chemical Formula 1, and may have a molecular weight of less than or equal to about 1,000 g/mol, for example, a molecular weight of less than or equal to about 900 g/mol, a molecular weight of less than or equal to about 800 g/mol, and a molecular weight of less than or equal to about 750 g/mol, for example, a molecular weight of about 250 g/mol to about 1,000 g/mol, about 300 g/mol to about 950 g/mol, etc. Therefore, the compound can be easily deposited and has an improved absorption ability for light in a long wavelength range, for example, greater than or equal to about 750 nm, for example greater than or equal to about 800 nm, for example greater than or equal to about 850 nm, for example greater than or equal to about 900 nm, for example greater than or equal to about 950 nm, for example greater than or equal to about 1,000 nm, greater than or equal to about 1,100 nm, greater than or equal to about 1,200 nm, or greater than or equal to about 1,300 nm. Accordingly, layers and/or structures comprising the compound as a near-infrared absorber may have improved sensitivity and/or absorbance to light at near-infrared wavelengths. A device (e.g., a sensor) configured to selectively absorb and/or convert near-infrared light (e.g., into an electrical signal, e.g., photoelectrically convert) may, for example, include a near-infrared absorber in an active layer configured to selectively absorb and/or convert the near-infrared light (convert into an electrical signal, for example, photoelectric conversion) to achieve improved performance and efficiency and thus may have improved functionality with regard to being configured to generate higher quality images and/or to generate images with reduced power consumption without compromising generated image quality.

The compound represented by Chemical Formula 1 is a novel compound that exhibits light absorption properties of absorbing light in the wavelength range of, for example, about 750 nm to about 3,000 nm, for example, a wavelength range of about 750 nm to about 2,500 nm, a wavelength range of about 800 nm to about 2,000 nm, a wavelength range of about 800 nm to about 1,800 nm, a wavelength range of about 800 nm to about 1,600 nm, a wavelength range of about 800 nm to about 1,500 nm, a wavelength range of about 800 nm to about 1,300 nm, a wavelength range of about 800 nm to about 1,200 nm, and/or a wavelength range of about 800 nm to about 1,000 nm.

In Chemical Formula 1, the electron-acceptor moiety may be represented by ((CN)₂—C═*, wherein * is a portion linked to the X or Y position of Chemical Formula 1 (e.g., a portion linked to a position of X in Chemical Formula 1 or a position of Y in Chemical Formula 1, a linking point with a position of X in Chemical Formula 1 or a position of Y in Chemical Formula 1, etc.).

In Chemical Formula 1, the electron-donor moiety may be represented by Chemical Formula 2:

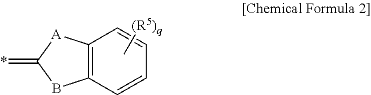

In Chemical Formula 2,
A and B may each independently be oxygen (O), sulfur (S), selenium (Se), or tellurium (Te),
R⁵ may be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q may be one of integers of 0 to 4, and
* may be a portion linked to the X or Y position of Chemical Formula 1 (e.g., a portion linked to a position of X in Chemical Formula 1 or a position of Y in Chemical Formula 1, a linking point with a position of X in Chemical Formula 1 or a position of Y in Chemical Formula 1, etc.).

In Chemical Formula 2, A and B may each independently be sulfur (S), selenium (Se), or tellurium (Te). For example, A and B may each independently be sulfur (S), or selenium (Se), and for example, A and B may both be sulfur (S).

In Chemical Formula 2, R⁵ may be deuterium, or a substituted or unsubstituted C1 to C10 saturated or unsaturated aliphatic hydrocarbon group. For example, in Chemical Formula 2, q may be 0, in which case, Chemical Formula 2 may not have a substituent R⁵.

In some example embodiments, Chemical Formula 2 may be represented by Chemical Formula 2-1, for example, Chemical Formula 3:

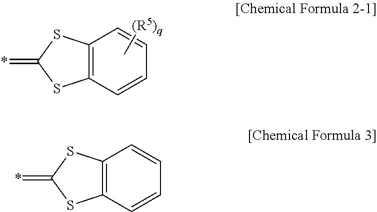

In Chemical Formula 2-1 and Chemical Formula 3,
R⁵, q, and * are each the same as R⁵, q, and *, respectively, as defined in Chemical Formula 2.

As an example of the electron-donor moiety and electron-acceptor moiety represented by the X and Y of Chemical Formula 1, the groups represented by chemical formula (CN)₂—C═* and Chemical Formula 2 are explained, respectively. However, the electron-donor moiety and electron-acceptor moiety present at both terminal ends (e.g., opposite terminal ends) of Chemical Formula 1 are not limited to these groups, and may include various types of electron-donor moieties and electron-acceptor moieties known in the art.

In some example embodiments, X and Y present at both terminal ends of Chemical Formula 1 may be both electron-donor moieties, X and Y may be both electron-acceptor moieties, or X may be an electron-donor moiety and Y may be an electron-acceptor moiety, or X may be an electron-acceptor moiety and Y may be an electron-donor moiety.

In Chemical Formula 1, R¹ to R⁴ may each independently be deuterium, a halogen, a cyano group, a nitro group, a substituted or unsubstituted C1 to C10 alkyl group, a substituted or unsubstituted C6 to C20 aryl group, or any combination thereof, and m and n may each independently be an integer of 0 or 1.

In Chemical Formula 1, p may be one of the integers from 1 to 4. When p is one of the integers of 1 to 4, it is advantageous to control the molecular weight of the compound represented by Chemical Formula 1 to less than or equal to about 1,000 g/mol. In this case, the compound represented by Chemical Formula 1 is advantageous for the deposition process.

In some example embodiments, when p in Chemical Formula 1 is 1, one of X or Y is an electron-acceptor moiety, and another one of X or Y is an electron-donor moiety, R¹ and R² may each independently be a halogen, a cyano group, a nitro group, a substituted or unsubstituted C2 to C20 alkyl group, a substituted or unsubstituted C6 to C20 aryl group, or any combination thereof. For example, R¹ and R² may each independently be a halogen, a cyano group, a nitro group, a substituted or unsubstituted C2 to C10 alkyl group, a substituted or unsubstituted C6 to C20 aryl group, or any combination thereof.

In some example embodiments, when p in Chemical Formula 1 is 1 and X and Y are the same electron-donor moiety (e.g., same electron-donor moiety, each a same electron-donor moiety, etc.), R¹ and R² in Chemical Formula 1 may each independently be hydrogen, a halogen, cyano group, nitro group, substituted or an unsubstituted C1 to C20 alkyl group, a substituted or unsubstituted C6 to C20 aryl group, or any combination thereof, but R¹ and R² are not simultaneously substituted or unsubstituted C1 to C20 alkyl groups (e.g., are not both substituted or unsubstituted C1 to C20 alkyl groups, are not each a simultaneously substituted or unsubstituted C1 to C20 alkyl group, are not simultaneously a substituted or unsubstituted C1 to C20 alkyl group).

In some example embodiments, when p of Chemical Formula 1 is 1, at least one of R¹ or R² may be a halogen, for example, one of R¹ or R² may be a halogen and the another one of R¹ or R² may be a substituted or unsubstituted C1 to C10 alkyl group, or both R¹ and R² may be a halogen. Herein, the halogen may be fluorine (F), and the substituted or unsubstituted C1 to C10 alkyl group may be an isopropyl group.

In some example embodiments, p in Chemical Formula 1 is 2, and in this case, X in Chemical Formula 1 may be represented by (CN)₂—C═* (wherein * is a portion linked to the X position of Chemical Formula 1, a linking point with the X position in Chemical Formula 1, etc.), and Y in Chemical Formula 1 may be represented by the following Chemical Formula 2:

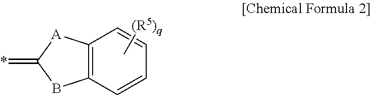

In Chemical Formula 2, A, B, R⁵, and q are the same as A, B, R⁵, and q, respectively, as defined in Chemical Formula 2. For example,
A and B may each independently be oxygen (O), sulfur (S), selenium (Se), or tellurium (Te),
R⁵ may be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q may be one of integers of 0 to 4, and
* may be a portion linked to the X or Y position of Chemical Formula 1 (e.g., a portion linked to a position of X in Chemical Formula 1 or a position of Y in Chemical Formula 1, a linking point with a position of X in Chemical Formula 1 or a position of Y in Chemical Formula 1, etc.).

In some example embodiments, p in Chemical Formula 1 is 2, and both X and Y in Chemical Formula 1 may be represented by Chemical Formula 2.

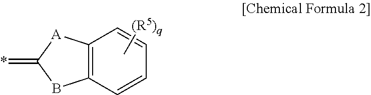

In Chemical Formula 2,
A and B are each independently oxygen (O), sulfur (S), selenium (Se), or tellurium (Te),
R⁵ is deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to a position of X in Chemical Formula 1 or a position of Y in Chemical Formula 1.

In Chemical Formula 1,
R¹ and R² may each independently be a halogen, a substituted or unsubstituted C1 to C5 alkyl group, or any combination thereof,
m and n are each independently 0 or 1,
p is one of integers of 1 to 4,
X and Y may each independently be (CN)₂—C═*,

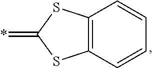 [SMILES: *=C1Sc2ccccc2S1]

or any combination thereof, wherein * is a portion linked to the X or Y position of Chemical Formula 1 (e.g., a position of X in Chemical Formula 1 or a position of Y in Chemical Formula 1),
when p is 1, at least one of R¹ or R² may be a halogen, and
when p is 2, X and Y may not be the same as each other (e.g., may be different from each other).

In some example embodiments, the halogen may be fluorine (F) or chlorine (Cl), and the C1 to C5 alkyl group may be a methyl group, an ethyl group, an n-propyl group, an isopropyl group, an n-butyl group, isobutyl, tert-butyl group, n-pentyl group, an isopentyl group, a tert-pentyl group, or any combination thereof.

In Chemical Formula 1,
when p is 3 or 4, X and Y may be the same or different from each other, and

R¹ and R² may each independently be hydrogen, a halogen, a substituted or unsubstituted C1 to C10 alkyl group, or any combination thereof.

Chemical Formula 1 may be represented by one or more of Chemical Formula 1-1 to Chemical Formula 1-3:

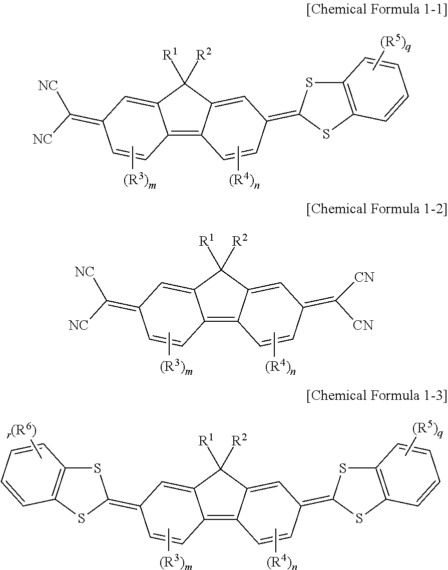

In Chemical Formulas 1-1 to 1-3,
R¹ and R² may each independently be hydrogen, deuterium, a halogen, or a substituted or unsubstituted C1 to C30 alkyl group,
R³ and R⁴ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
m and n may each independently be one of integers of 0 to 3,
R⁵ and R⁶ may each independently be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q and r may each independently be one of integers from 0 to 4,

In Chemical Formula 1-1, R¹ and R² may not be simultaneously hydrogens or methyl groups (e.g., may not be simultaneously hydrogen and may not be simultaneously a methyl group, may not simultaneously be hydrogen or a methyl group, etc.), and
in Chemical Formula 1-2, R¹ and R² may not be simultaneously substituted or unsubstituted C1 to C30 alkyl groups (may not simultaneously be a substituted or unsubstituted C1 to C30 alkyl group).

In Chemical Formulas 1-1 to 1-3,
R¹ and R² may each independently be a halogen, for example fluorine (F), or may be a substituted or unsubstituted C1 to C5 alkyl group, wherein at least one of R¹ and R² is halogen, for example fluorine (F),
R³ to R⁶ may each independently be deuterium, a halogen, a hydroxy group, a cyano group, a nitro group, a substituted or unsubstituted C1 to C5 alkyl group, a substituted or unsubstituted C3 to C10 cycloalkyl group, a substituted or unsubstituted C6 to C10 aryl group, or any combination thereof, and

m, n, q, and r may each independently be one of the integers from 0 to 2.

In Chemical Formulas 1-1 to 1-3, R¹ and R² are each independently fluorine or a C1 to C5 alkyl group, wherein at least one of R¹ or R² may be fluorine, and m, n, q, and r may be all 0.

Chemical Formula 1 may be represented by Chemical Formula 1-4 or Chemical Formula 1-5:

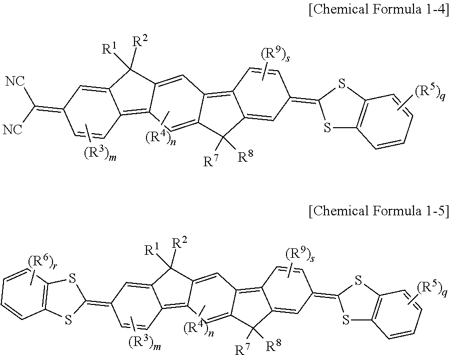

In Chemical Formula 1-4 and Chemical Formula 1-5,
R¹, R², R⁷, and R⁸ may each independently be hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³, R⁴, and R⁹ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R⁵ and R⁶ may each independently be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
m and s may each independently be one of integers of 0 to 3,
n is one of integers of 0 to 2, and
q and r may each independently be one of integers of 0 to 4.

In Chemical Formula 1-4 and Chemical Formula 1-5,
R¹, R², R⁷, and R^{a} may each independently be hydrogen, deuterium, a halogen, a hydroxy group, a cyano group, a nitro group, a substituted or unsubstituted C1 to C10 alkyl group, a substituted or unsubstituted C3 to C10 cycloalkyl group, a substituted or unsubstituted C6 to C10 aryl group, a substituted or unsubstituted C3 to C30 heteroaryl group, a substituted or unsubstituted C1 to C30 alkoxy group, a substituted or unsubstituted C7 to C30 aryloxy group, or any combination thereof,
R³, R⁴, and R⁹ may each independently be deuterium, a halogen, a hydroxy group, a cyano group, a nitro group, a substituted or unsubstituted C1 to C10 alkyl group, a substituted or unsubstituted C3 to C10 cycloalkyl group, a substituted or unsubstituted C6 to C10 aryl group, a substituted or unsubstituted C3 to C30 heteroaryl group, a substituted or unsubstituted C1 to C30 alkoxy group, a substituted or unsubstituted C7 to C30 aryloxy group, or any combination thereof,
R⁵ and R⁶ may each independently be deuterium, a substituted or unsubstituted C1 to C10 alkyl group, a substituted or unsubstituted C3 to C10 cycloalkyl group, a substituted or unsubstituted C6 to C30 aryl group, a substituted or unsubstituted C3 to C30 heteroaryl group, or any combination thereof,
m and s may each independently be one of integers of 0 to 2,
n may be 0 or 1,
q and r may each independently be one of the integers of 0 to 2.

In Chemical Formula 1-4 and Chemical Formula 1-5,
R¹, R², R⁷, and R^{a} may each independently be hydrogen, deuterium, a halogen, a substituted or unsubstituted C1 to C5 alkyl group, a substituted or unsubstituted C3 to C6 cycloalkyl group, a substituted or unsubstituted C6 to C10 aryl group, or any combination thereof,
R³, R⁴, R⁵, R⁶, and R⁹ may each independently be deuterium, a substituted or unsubstituted C1 to C5 alkyl group, a substituted or unsubstituted C3 to C6 cycloalkyl group, a substituted or unsubstituted C6 to C10 aryl group, or any combination thereof, and
m, n, s, q, and r may each independently be an integer of 0 or 1.

In Chemical Formula 1-4 and Chemical Formula 1-5,
R¹, R², R⁷, and R⁸ may each independently be hydrogen, deuterium, a halogen, a substituted or unsubstituted C1 to C5 alkyl group, or any combination thereof, and
m, n, s, q, and r may be all 0 (e.g., may each be 0).

Chemical Formula 1 may be represented by Chemical Formula 1-6 or Chemical Formula 1-7:

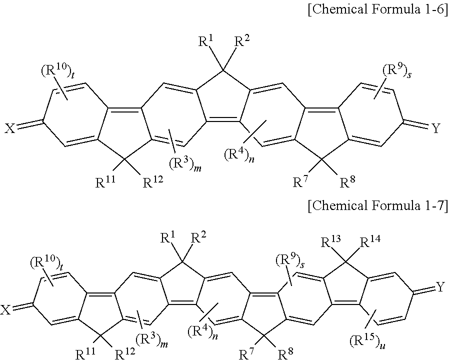

In Chemical Formula 1-6 and Chemical Formula 1-7,
R¹, R², R⁷, R⁸, R¹¹, R¹², R¹³, and R¹⁴ may each independently be hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³, R⁴, R⁹, R¹⁰, and R¹⁵ may each independently be deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
X and Y may each independently be an electron-acceptor moiety, an electron-donor moiety, or any combination thereof,
in Chemical Formula 1-6, m and n may each independently be one of integers of 0 to 2, and s and t may each independently be one of integers of 0 to 3, and
in Chemical Formula 1-7, m, n, and s may each independently be one of integers of 0 to 2, and t and u may each independently be one of integers of 0 to 3.

In Chemical Formula 1-6 and Chemical Formula 1-7, the electron-acceptor moiety may be represented by (CN)₂—C═*, wherein * is a portion linked to the X or Y position of Chemical Formula 1-6 or Chemical Formula 1-7 (e.g., a position of X in Chemical Formula 1-6 or Chemical Formula 1-7 or a position of Y in Chemical Formula 1-6 or Chemical Formula 1-7), and the electron-donor moiety may be represented by Chemical Formula 2:

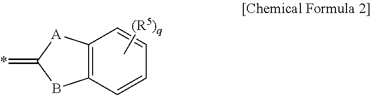

wherein, in Chemical Formula 2,
A and B may each independently be oxygen (O), sulfur (S), selenium (Se), or tellurium (Te),
R⁵ may be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q may be one of integers of 0 to 4,
   * is a portion linked to (e.g., a linking point with) the X or Y position of Chemical Formula 1-6 or Chemical Formula 1-7 (e.g., the position of X in Chemical Formula 1-6 or Chemical Formula 1-7 or the position of Y in Chemical Formula 1-6 or Chemical Formula 1-7).

In Chemical Formula 1-6 and Chemical Formula 1-7, the X may be represented by (CN)₂—C═*, wherein * is a portion linked to the X or Y position of Chemical Formula 1-6 or Chemical Formula 1-7, and the Y may be represented by Chemical Formula 2-1:

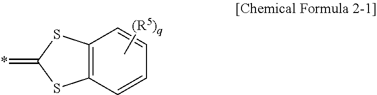

In Chemical Formula 2-1,
R⁵ may be deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q may be one of the integers from 0 to 4, and
* is a portion linked to the Y position of Chemical Formula 1-6 or Chemical Formula 1-7.

In Chemical Formula 1-6 and Chemical Formula 1-7, both X and Y may be represented by (CN)₂—C═*, wherein * is a portion linked to the X or Y position of Chemical Formula 1-5 or Chemical Formula 1-6, or both X and Y may be represented by Chemical Formula 2-1:

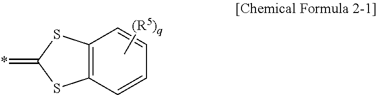

R⁵, q, and * in Chemical Formula 2-1 are the same as R⁵, q, and *, respectively, as defined above.

In Chemical Formula 1-6 and Chemical Formula 1-7,
R¹, R², R⁷, R⁸, R¹¹, R¹², R¹³, and R¹⁴ may each independently be hydrogen, deuterium, a halogen, a hydroxy group, a cyano group, a nitro group, a substituted or unsubstituted C1 to C10 alkyl group, a substituted or unsubstituted C3 to C10 cycloalkyl group, a substituted or unsubstituted C6 to C10 aryl group, a substituted or unsubstituted C3 to C10 heteroaryl group, a substituted or unsubstituted C1 to C10 alkoxy group, a substituted or unsubstituted C7 to C10 aryloxy group, or any combination thereof,
R³, R⁴, R⁹, R¹⁰, and R¹⁵ may each independently be deuterium, a halogen, a hydroxy group, a cyano group, a nitro group, a substituted or unsubstituted C1 to C10 alkyl group, a substituted or unsubstituted C3 to C10 cycloalkyl group, a substituted or unsubstituted C6 to C10 aryl group, a substituted or unsubstituted C3 to C10 heteroaryl group, a substituted or unsubstituted C1 to C10 alkoxy group, a substituted or unsubstituted C7 to C10 aryloxy group, or any combination thereof,
X and Y may each independently be (CN)₂—C═* (wherein, * is a portion linked to the X or Y position of Chemical Formula 1-5 or Chemical Formula 1-6), or represented by Chemical Formula 2-1, and
m, n, s, t, and u may each independently be one of the integers of 0 to 2.

In Chemical Formula 1-6 and Chemical Formula 1-7,
R¹, R², R⁷, R⁸, R¹¹, R¹², R¹³, and R¹⁴ may each independently be hydrogen, deuterium, a halogen, a substituted or unsubstituted C1 to C5 alkyl group, a substituted or unsubstituted C3 to C6 cycloalkyl group, a substituted or unsubstituted C6 to C10 aryl group, a substituted or unsubstituted C3 to C10 heteroaryl group, a substituted or unsubstituted C1 to C5 alkoxy group, a substituted or unsubstituted C7 to C10 aryloxy group, or any combination thereof,
R³, R⁴, R⁹, R¹⁰, and R¹⁵ may each independently be deuterium, a halogen, a substituted or unsubstituted C1 to C5 alkyl group, a substituted or unsubstituted C3 to C6 cycloalkyl group, a substituted or unsubstituted C6 to C10 aryl group, a substituted or unsubstituted C3 to C10 heteroaryl group, a substituted or unsubstituted C1 to C5 alkoxy group, a substituted or unsubstituted C7 to C10 aryloxy group, or any combination thereof,
X and Y may each independently be (CN)₂—C═*, wherein, * is a portion linked to the X or Y position of Chemical Formula 1-5 or Chemical Formula 1-6, or may be represented by Chemical Formula 2-1, and
m, n, s, t, and u may each independently be an integer of 0 or 1.

In Chemical Formula 1-6 and Chemical Formula 1-7,
R¹, R², R⁷, R⁸, R¹¹, R¹², R¹³, and R¹⁴ may each independently be hydrogen, deuterium, a halogen, a substituted or unsubstituted C1 to C5 alkyl group, a substituted or unsubstituted C3 to C6 cycloalkyl group, a substituted or unsubstituted C6 to C10 aryl group, a substituted or unsubstituted C3 to C10 heteroaryl group, or any combination thereof,
X and Y may each independently be (CN)₂—C═* (wherein, * is a portion linked to the X or Y position of Chemical Formula 1-5 or Chemical Formula 1-6), or may be represented by Chemical Formula 2-1, and
m, n, s, t, and u may be all 0.

In Chemical Formula 1-1 to Chemical Formula 1-7, the halogen may be fluorine (F) or chlorine (Cl), and the C1 to C5 alkyl group may be a methyl group, an ethyl group, an n-propyl group, an isopropyl group, an n-butyl group, isobutyl, tert-butyl group, n-pentyl group, an isopentyl group, a tert-pentyl group, or any combination thereof.

Examples of the compounds according to some example embodiments include the following compounds, but are not limited thereto:

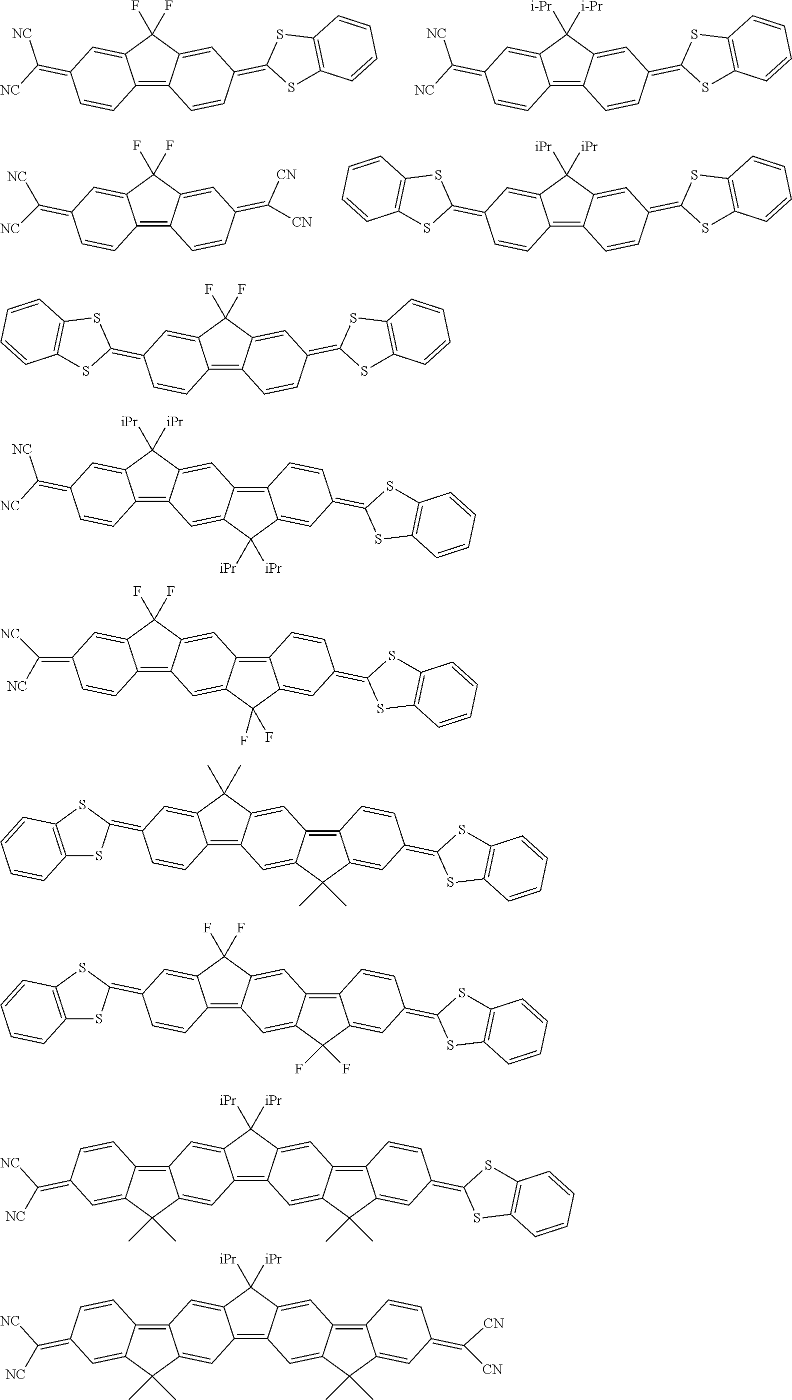

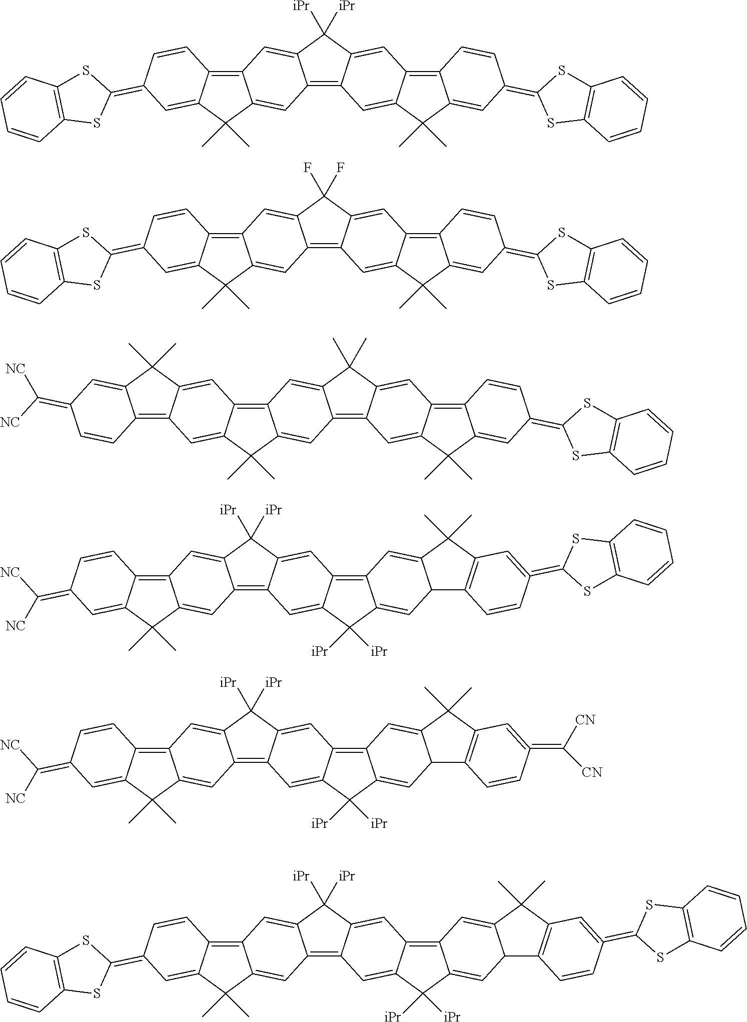

As described above, the molecular weight of the compound according to some example embodiments may be in the range of about 250 g/mol to about 1,000 g/mol, which is an example range when the compound is applied using a deposition process. The molecular weight range of the compound is not necessarily limited to the above range.

In addition, a peak absorption wavelength (λmax) of the compound may be greater than or equal to about 750 nm, for example greater than or equal to about 770 nm, for example greater than or equal to about 780 nm, for example greater than or equal to about 790 nm, for example greater than or equal to about 800 nm, for example greater than or equal to about 810 nm, for example greater than or equal to about 820 nm, or for example greater than or equal to about 830 nm. For example, the peak absorption wavelength of the compound may be in the wavelength region of, e.g., about 750 nm to about 3,000 nm, within this range, e.g., about 750 nm to about 2,500 nm, for example about 780 nm to about 2,200 nm, for example about 790 nm to about 2,100 nm, for example about 800 nm to about 2,000 nm, for example about 810 nm to about 2,000 nm, for example about 820 nm to about 2,000 nm or for example about 830 nm to about 2,000 nm, for example, about 850 nm to about 2,000 nm, for example, about 900 nm to about 2,000 nm, for example, about 950 nm to about 1,800 nm, for example, about 1,000 nm to about 1,800 nm, for example, about 1,100 nm to about 1,800 nm, for example, about 1,150 nm to about 1,800 nm, for example, about 1,200 nm to about 1,800 nm, for example, about 1,250 nm to about 1,700 nm, for example, about 1,300 nm to about 1,700 nm, for example, about 1,300 nm to about 1,600 nm, for example, about 1,300 nm to about 1,500 nm.

Therefore, the compound according to some example embodiments may be configured to absorb light in the near-infrared wavelength region.

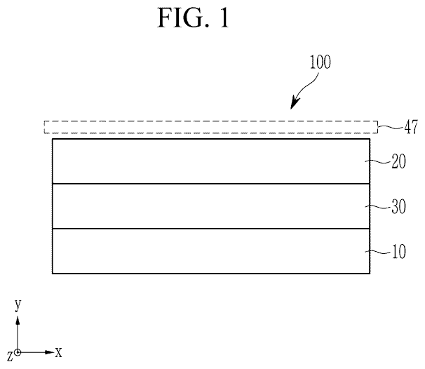

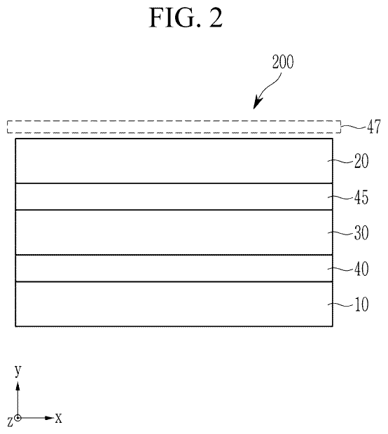

In addition, the compound according to some example embodiments may exhibit good charge transfer characteristics, and thus has good photoelectric conversion characteristics of absorbing (e.g., selectively absorbing) light and converting it (the absorbed light) into an electrical signal (e.g., photoelectric conversion), and thus it (the compound) may be effectively used as a photoelectric conversion material for photoelectric devices. Therefore, for example, a photovoltaic device including the compound according to some example embodiments may be applied to the active layer and/or charge auxiliary layer (e.g., active layer 30 and/or charge auxiliary layer 40, 45 shown in FIGS. 1 and 2) of a photovoltaic device may have improved performance and/or efficiency, and for example, may have improved performance and/or efficiency with respect to implementing photoelectric conversion of incident near-infrared rays by including the compound.

The compound according to some example embodiments has good heat resistance, and thus thermal decomposition can be prevented or reduced during deposition, and can be deposited repeatedly. The compound may be thermally or vacuum deposited, for example by sublimation. For example, deposition by sublimation can be confirmed by thermogravimetric analysis (TGA), and when thermogravimetric analysis is performed at a pressure of less than or equal to about 10 Pascal (Pa), for example, a weight loss of 10% compared to the initial weight occurs. The temperature may be less than or equal to about 400° C., for example, less than or equal to about 390° C., less than or equal to about 380° C., less than or equal to about 370° C. For example, when the compound is subjected to thermogravimetric analysis at a pressure of less than or equal to about 10 Pa, the temperature at which a weight loss of 10% compared to an initial weight occurs may be about 230° C. to about 400° C.

Some example embodiments provide a near-infrared absorbing/blocking film including the compound according to the example embodiments (e.g., the compound represented by Chemical Formula 1).

The near-infrared absorbing/blocking film may be applied to (e.g., included in) various fields that require light absorption characteristics (e.g., peak absorption wavelength) in the near-infrared wavelength region.

The compound according to some example embodiments has both light absorption characteristics and photoelectric characteristics in the near-infrared wavelength region, and thus can be effectively used as a photoelectric conversion material.

Hereinafter, a photoelectric device, an image sensor, and an electronic device including a compound according to some example embodiments (e.g., the compound represented by Chemical Formula 1) will be described with reference to the drawings.

FIG. 1 is a cross-sectional view illustrating an photoelectronic device according to some example embodiments.

Referring to FIG. 1, a photoelectric device 100 according to some example embodiments includes a first electrode 10 and a second electrode 20 facing each other, and an active layer 30 disposed between the first electrode 10 and the second electrode 20.

A substrate (not shown) may be disposed on the first electrode 10 side or on the second electrode 20 side. The substrate may be for example an inorganic substrate such as a glass plate or silicon wafer or an organic substrate made of an organic material such as polycarbonate, polymethylmethacrylate, polyethyleneterephthalate, polyethylenenaphthalate, polyamide, polyethersulfone, or any combination thereof. The substrate may be omitted.

One of the first electrode 10 or the second electrode 20 may be an anode and the other may be a cathode. For example, the first electrode 10 may be an anode and the second electrode 20 may be a cathode.

At least one of the first electrode 10 or the second electrode 20 may be a light-transmitting electrode and the light-transmitting electrode may be for example made of a conductive oxide such as an indium tin oxide (ITO), indium zinc oxide (IZO), zinc oxide (ZnO), tin oxide (SnO), aluminum tin oxide (AITO), and/or fluorine doped tin oxide (FTO), or a metal thin layer of a single layer or a multilayer. When one of the first electrode 10 or the second electrode 20 is a non-light-transmitting electrode, it may be made of, for example, an opaque conductor such as aluminum (Al), silver (Ag), or gold (Au). For example, the first electrode 10 and the second electrode 20 may be all light-transmitting electrodes. For example, the second electrode 20 may be a light receiving electrode disposed at a light receiving side.

The active layer 30 is a layer including a p-type semiconductor and an n-type semiconductor configured to provide a pn junction, which is a layer that may produce excitons by receiving light from outside (e.g., an exterior of the active layer 30) and then separating holes and electrons from the produced excitons.

The p-type semiconductor and the n-type semiconductor may be independently a light absorbing material that is configured to absorb (e.g., selectively absorb) light in at least one portion of a wavelength region and the compound according to some example embodiments may be a p-type semiconductor or an n-type semiconductor. For example, the aforementioned compound according to some example embodiments may be used (e.g., included in the active layer 30) as a p-type semiconductor and fullerene or a fullerene derivative may be included (e.g., included in the active layer 30) as an n-type semiconductor. Accordingly, it will be understood that the active layer 30 may at least partially comprise the aforementioned compound according to some example embodiments, for example as a near-infrared absorber (e.g., may include the compound according to some example embodiments and may further include either fullerene or a fullerene derivative). Additionally, the active layer 30 may have a peak absorption wavelength (λmax) of, for example, greater than or equal to about 750 nm, greater than or equal to about 770 nm, greater than or equal to about 780 nm, greater than or equal to about 790 nm, greater than or equal to about 800 nm, greater than or equal to about 810 nm, greater than or equal to about 820 nm, or greater than or equal to about 830 nm. The peak absorption wavelength of the aforementioned compound, for example where the aforementioned compound is included in the active layer 30 as a near-infrared absorber, may fall within a wavelength region of, for example, about 750 nm to about 3000 nm, and within this range, for example, about 750 nm to about 2500 nm, for example about 780 nm to about 2200 nm, for example about 790 nm to about 2100 nm, for example about 800 nm to about 2000 nm, for example about 810 nm to about 2000 nm, for example about 820 nm to about 2000 nm or for example about 830 nm to about 2000 nm. The active layer 30, and thus the photoelectric device 100 may have improved near-infrared light absorption characteristics (e.g., may have improved sensitivity to light in a near-infrared wavelength region, improved absorbance of light in the near-infrared wavelength region, a peak absorbance wavelength in the near-infrared wavelength region, etc.) and thus improved photoelectric conversion performance and/or efficiency (e.g., configuration to perform photoelectric conversion of incident light with reduced power consumption and without compromising photoelectric conversion performance) and/or improved thermal stability based on the active layer including the aforementioned compound (e.g., the compound represented by Chemical Formula 1) according to some example embodiments.

In some example embodiments, the active layer 30 may be a near-infrared absorbing/blocking film that includes the compound according to some example embodiments. The active layer 30 may include an intrinsic layer in which the aforementioned near-infrared absorber (e.g., p-type semiconductor) and fullerene or a fullerene derivative (e.g., n-type semiconductor) are co-deposited. Herein, the p-type semiconductor and the n-type semiconductor may be included in a volume ratio of about 1:9 to about 9:1, for example about 2:8 to about 8:2, about 3:7 to about 7:3, about 4:6 to about 6:4, or about 5:5.

The active layer 30 may further include a p-type layer and/or an n-type layer in addition to the intrinsic layer. The p-type layer may include the aforementioned compound according to some example embodiments, which may serve as a near-infrared absorber (e.g., p-type semiconductor) described above, and the n-type layer may include the n-type semiconductor described above. For example, the active layer 30 may include the aforementioned compound according to some example embodiments and the aforementioned n-type semiconductor in various combinations of p-type layer/I layer, I layer/n-type layer, p-type layer/I layer/n-type layer, and the like.

The photoelectric device 100 may further include an auxiliary layer (not shown) between the first electrode 10 and the active layer 30 and/or between the second electrode 20 and the active layer 30. The auxiliary layer may be a charge auxiliary layer or an optical auxiliary layer. Such a photoelectric device is shown in FIG. 2.

FIG. 2 is a cross-sectional view showing a photoelectric device according to some example embodiments. Referring to FIG. 2, the photoelectric device 200 includes a first electrode 10 and a second electrode 20 facing each other, and an active layer 30 between the first electrode 10 and the second electrode 20, a first auxiliary layer 40 between the first electrode 10 and the active layer 30, and a second auxiliary layer 45 between the second electrode 20 and the active layer 30. In some example embodiments, one alone of the first auxiliary layer 40 or the second auxiliary layer 45 may be included in the photoelectric device 200 and the other one of the of the first auxiliary layer 40 or the second auxiliary layer 45 may be omitted from the photoelectric device 200.

The first auxiliary layer 40 and the second auxiliary layer 45 are charge auxiliary layers, and can facilitate the movement of holes and electrons separated from the active layer 30 to increase efficiency. The first auxiliary layer 40 and the second auxiliary layer 45 may each be referred to herein interchangeably as a charge auxiliary layer.

The charge auxiliary layers 40 and/or 45 may be at least one selected from a hole injection layer (HIL) for facilitating hole injection, a hole transport layer (HTL) for facilitating hole transport, an electron blocking layer (EBL) for preventing electron transport, an electron injection layer (EIL) for facilitating electron injection, an electron transport layer (ETL) for facilitating electron transport, and a hole blocking layer (HBL) for preventing hole transport.

The charge auxiliary layers 40 and/or 45 may include, for example, an organic material, an inorganic material, or an organic/inorganic material. The organic material may be an organic compound having hole or electron characteristics, and the inorganic material may be, for example, a metal oxide such as molybdenum oxide, tungsten oxide, nickel oxide, and the like.

The charge auxiliary layer may include, for example, the compound according to some example embodiments (e.g., the compound represented by Chemical Formula 1). In some example embodiments, the charge auxiliary layers 40 and/or 45 may include the compound according to some example embodiments, and the active layer 30 may also include the compound according to some example embodiments. In some example embodiments, the charge auxiliary layers 40 and/or 45 may include the compound according to some example embodiments, and the active layer 30 may not include the compound according to some example embodiments. The charge auxiliary layers 40 and/or 45, and thus the photoelectric device 200, may have improved near-infrared light absorption characteristics (e.g., may have improved sensitivity to light in a near-infrared wavelength region, improved absorbance of light in the near-infrared wavelength region, etc.) and thus improved photoelectric conversion performance and/or efficiency, and/or improved thermal stability based on the charge auxiliary layers 40 and/or 45 including the compound according to some example embodiments.

The optical auxiliary layer may be disposed in a light incident direction of the photoelectric device and may be for example disposed on the active layer 30 when the second electrode 20 is a light receiving electrode (e.g., an electrode close to the position where light enters the photoelectric device 200). For example, the optical auxiliary layer may be disposed between the second electrode 20 and the active layer 30.

The photoelectric devices 100 and 200 may further include an anti-reflection layer 47 on one surface of the first electrode 10 or the second electrode 20. The anti-reflection layer 47 is disposed at a light incidence side and lowers reflectance of light of incident light and thereby light absorbance is further improved. For example, when light is incident on the first electrode 10, the anti-reflection layer may be disposed on (e.g., disposed at an exposed surface of) one surface of the first electrode 10, and when light is incident on the second electrode 20, the anti-reflection layer may be disposed on (e.g., disposed at an exposed surface of) one surface of the second electrode 20.

The anti-reflection layer 47 may include, for example a material having a refractive index of about 1.6 to about 2.5 and may include for example at least one of a metal oxide, a metal sulfide, and an organic material having a refractive index within the above ranges. The anti-reflection layer 47 may include, for example a metal oxide such as an aluminum-containing oxide, a molybdenum-containing oxide, a tungsten-containing oxide, a vanadium-containing oxide, a rhenium-containing oxide, a niobium-containing oxide, a tantalum-containing oxide, a titanium-containing oxide, a nickel-containing oxide, a copper-containing oxide, a cobalt-containing oxide, a manganese-containing oxide, a chromium-containing oxide, a tellurium-containing oxide, or any combination thereof; a metal sulfide such as zinc sulfide; or an organic material such as an amine derivative, but is not limited thereto.

When light is incident from the first electrode 10 or the second electrode 20 and the active layer 30 absorbs light in a specific (or predetermined) wavelength range, excitons are generated inside the photoelectric devices 100 and 200. The excitons are separated into holes and electrons in the active layer 30, and the separated holes are transported to an anode that is one of the first electrode 10 and the second electrode 20 and the separated electrons are transported to the cathode that is the other of the first electrode 10 and the second electrode 20 so as to flow a current.

The photoelectric devices 100 and 200 may be applied to (e.g., included in) a solar cell, an image sensor, a photodetector, a photosensor, and an organic light emitting diode (OLED), but are not limited thereto.

The photoelectric devices 100 and 200 may be applied to an organic sensor. The organic sensor may be an organic CMOS sensor, for example, an organic CMOS infrared light sensor or an organic CMOS image sensor. In some example embodiments, the photoelectric device 100 may include the compound according to some example embodiments in any of the elements thereof, including, in addition to or alternative to the active layer 30, one or more of the first electrode 10 or the second electrode 20. In some example embodiments, the photoelectric device 200 may include the compound according to some example embodiments in any of the elements thereof, including, in addition to or alternative to the active layer 30 and/or one or more of the charge auxiliary layers 40/45, one or more of the first electrode 10 or the second electrode 20.

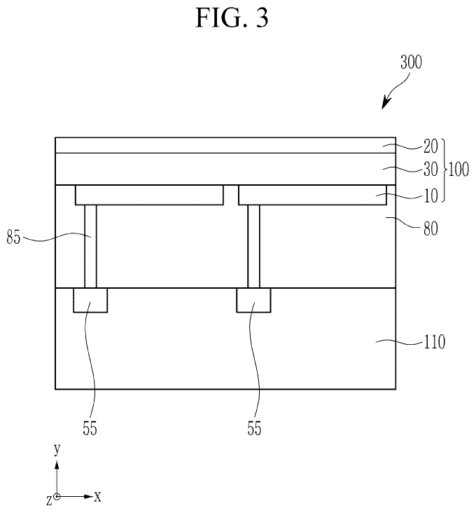

FIG. 3 is a cross-sectional view showing an organic sensor according to some example embodiments. As described herein, an organic sensor according to any of the example embodiments may be an image sensor.

The organic sensor 300 according to some example embodiments includes a semiconductor substrate 110, an insulation layer 80, and a photoelectric device 100.

The semiconductor substrate 110 may be a silicon substrate, and a transmission transistor (not shown) and a charge storage 55 are integrated therein. The charge storage 55 may be integrated for each pixel. The charge storage 55 is electrically connected to the photoelectric device 100, and information in the charge storage 55 may be transferred by a transmission transistor.

A metal wire (not shown) and a pad (not shown) are formed on the semiconductor substrate 110. In order to decrease signal delay, the metal wire and pad may be made of a metal having low resistivity, for example, aluminum (Al), copper (Cu), silver (Ag), and alloys thereof, but is not limited thereto. However, the structure is not limited thereto, and the metal wire and pad may be disposed under the semiconductor substrate 110.

An insulation layer 80 is formed on the metal wire and pad. The insulation layer 80 may be made of an inorganic insulating material such as a silicon oxide and/or a silicon nitride, or a low dielectric constant (low K) material such as SiC, SiCOH, SiCO, and/or SiOF. The insulation layer 80 has a trench 85 exposing the charge storage 55. The trench may be filled with fillers.

The aforementioned photoelectric device 100 is formed on the insulation layer 80. As described above, the photoelectric device 100 includes a first electrode 10, an active layer 30, and a second electrode 20. Even though a structure in which the first electrode 10, the active layer 30, and the second electrode 20 are sequentially stacked is shown as an example in the drawing, the present inventive concepts are not limited to this structure, and the second electrode 20, the active layer 30, and the first electrode 10 may be arranged in this order.

Both the first electrode 10 and the second electrode 20 may be transparent electrodes, and the active layer 30 is as described with reference to FIGS. 1 and 2. The active layer 30 can selectively absorb light in the near-infrared wavelength region. Incident light from the side of the second electrode 20 may be photoelectrically converted by mainly absorbing light in a near-infrared wavelength region in the active layer 30. As noted above with reference to FIG. 1, the active layer 30 may include the compound according to some example embodiments and thus may have improved sensitivity to and/or improved absorbance of near-infrared light, such that the operational performance and/or efficiency of the organic sensor 300 in absorbing and/or converting incident near-infrared light into electrical signals (e.g., photoelectric conversion performance and/or efficiency) may be improved. As a result, the functionality of the organic sensor 300 with regard to sensing (e.g., absorbing and/or photoelectrically converting incident light in the near-infrared wavelength region) may be improved based on including the compound in the active layer 30.

Focusing lens (not shown) may be further formed on the photoelectric device 100. The focusing lens may control a direction of incident light and gather the light in one region. The focusing lens may have a shape of, for example, a cylinder or a hemisphere, but is not limited thereto.

Although the organic sensor to which the photoelectric device 100 of FIG. 1 is applied is illustrated in FIG. 3, the photoelectric device 200 according to FIG. 2 may be equally applied (e.g., included in place of photoelectric device 100 in the organic sensor 300).

The organic sensor according to some example embodiments may be an organic infrared light sensor, for example an iris sensor or a depth sensor.

The iris sensor identifies a person by using unique iris characteristics of every person and specifically, taking an image of an eye of a user within an appropriate distance, processing the image, and comparing it with his/her stored image.

The depth sensor identifies a shape and a location of an object from its three-dimensional information by taking an image of the object within an appropriate distance with a user and processing the image. This depth sensor may be for example used as a face recognition sensor.

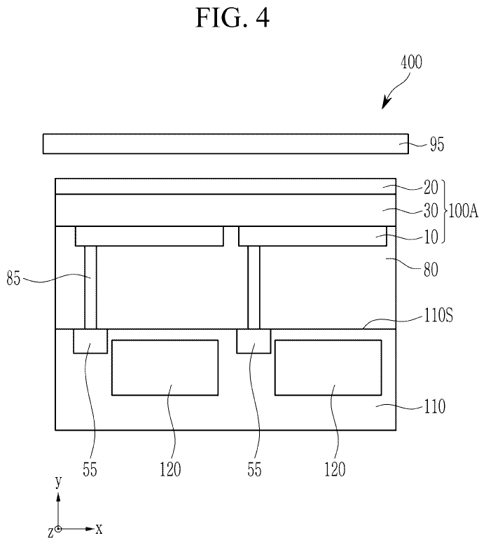

FIG. 4 is a cross-sectional view showing an organic sensor according to some example embodiments.

The organic sensor according to some example embodiments may include a plurality of sensors having different functions. For example, at least one of the plurality of sensors having different functions may be a biometric sensor, and the biometric sensor may be for example an iris sensor, a depth sensor, a fingerprint sensor, a blood vessel distribution sensor, and the like, but is not limited thereto. As an example, one of the plurality of sensors with different functions may be an iris sensor and the other may be a distance sensor.

A plurality of sensor may include, for example, a first infrared light sensor configured to sense (e.g., selectively absorb and/or convert (convert into an electrical signal, e.g., photoelectric conversion)) light in an infrared region having a first wavelength (λ1) in an infrared wavelength region and a second infrared light sensor configured to sense (e.g., selectively absorb and/or convert (convert into an electrical signal, e.g., photoelectric conversion)) light in an infrared region having a second wavelength (λ2) (an infrared wavelength region that is the same as or different from the infrared wavelength region including the first wavelength (λ1)) in an infrared wavelength region.

The first wavelength (λ1) and the second wavelength (λ2) may be for example different in a wavelength region of about 750 nm to about 3000 nm, and for example a difference between the first wavelength (λ1) and the second wavelength (λ2) may be greater than or equal to about 30 nm, greater than or equal to about 50 nm, greater than or equal to about 70 nm, greater than or equal to about 80 nm, or greater than or equal to about 90 nm.

For example, one of the first wavelength (λ1) or the second wavelength (λ2) may belong to a wavelength region of about 780 nm to about 900 nm and the other of the first wavelength (λ1) or the second wavelength (λ2) may belong to a wavelength region of greater than about 900 nm and less than or equal to about 1000 nm.

For example, one of the first wavelength (λ1) or the second wavelength (λ2) may belong to a wavelength region of about 780 nm to about 840 nm and the other of the first wavelength (λ1) or the second wavelength (λ2) may belong to a wavelength region of about 910 nm to about 970 nm.

For example, one of the first wavelength (λ1) or the second wavelength (λ2) may belong to a wavelength range of about 800 nm to about 830 nm, and the other of the first wavelength (λ1) or the second wavelength (λ2) may belong to a wavelength range of about 930 nm to about 950 nm.

For example, one of the first wavelength (λ1) or the second wavelength (λ2) may belong to a wavelength range of about 805 nm to about 815 nm, and the other of the first wavelength (λ1) or the second wavelength (λ2) may belong to a wavelength range of about 935 nm to about 945 nm.

For example, one of the first wavelength (λ1) or the second wavelength (λ2) may be about 810 nm, and the other of the first wavelength (λ1) or the second wavelength (λ2) may be about 940 nm.

The organic sensor 400 according to some example embodiments includes a dual bandpass filter 95, a first infrared light sensor 100A, an insulating layer 80, and a semiconductor substrate 110 integrated with a second infrared light sensor 120. The second infrared light sensor 120 may be at least partially embedded within the semiconductor substrate 110. As shown in FIG. 4, the first infrared light sensor 100A and the second infrared light sensor 120 may be stacked, e.g., may overlap in a vertical direction that is perpendicular to the upper surface 110S of the semiconductor substrate 110.

As shown in FIG. 4, dual bandpass filter 95 may be disposed on a front side of the organic sensor 400, and may selectively transmit infrared light including the first wavelength (λ1) (e.g., light in the infrared wavelength region) and infrared light including the second wavelength (λ2) and may block and/or absorb other light. Herein, other light may include light in an ultraviolet (UV) and visible region.

The first infrared light sensor 100A includes a first electrode 10, an active layer 30, and a second electrode 20. As shown in FIG. 4, the first infrared light sensor 100A may be the same as the photoelectric device 100 of some example embodiments, including the example embodiments described above with reference to FIG. 1. In some example embodiments, the first infrared light sensor 100A may be the photoelectric device 200 shown in FIG. 2.

As shown in FIG. 4, the second infrared light sensor 120 may be integrated within the semiconductor substrate 110 and may be a light sensing device. The semiconductor substrate 110 may be for example a silicon substrate and may be integrated with the second infrared light sensor 120, the charge storage 55, and a transmission transistor (not shown).

The second infrared light sensor 120 may be a photodiode (e.g., silicon-based photodiode) and may sense (e.g., absorb) entered light, and sensed information is transferred by the transmission transistor. Herein, the light entered into the second infrared light sensor 120 is light that passes (e.g., selectively transmits) the dual bandpass filter 95 and the first infrared light sensor 100A and may be infrared light in a certain (or predetermined) region including the second wavelength (λ2). All infrared light in a predetermined region including the first wavelength (λ1) may be absorbed by the active layer 30 and may not reach the second infrared light sensor 120. In this case, a separate filter for wavelength selectivity with respect to the light entered into the second infrared light sensor 120 is not separately needed and may be omitted from the organic sensor 400. However, for the time when all infrared light in a certain (predetermined) region including the first wavelength (λ1) is not absorbed by the active layer 30, a filter between the first infrared light sensor 100A and the second infrared light sensor 120 may be further disposed.

Accordingly, in the organic sensor 400, the first infrared light sensor 100A may be understood to include a photoelectric device (e.g., photoelectric device 100 and/or 200) configured to sense (e.g., selectively absorb and/or convert into electrical signals (e.g., photoelectrically convert)) light in a first near-infrared wavelength region of incident light (e.g., a first near-infrared wavelength region including the first wavelength (λ1)), and the second infrared light sensor 120 may be understood to be an additional sensor configured to selectively absorb and/or convert into electrical signals (e.g., photoelectrically convert) light in a separate wavelength region of incident light (e.g., a second near-infrared wavelength region that is different from the first near-infrared wavelength region and includes the second wavelength (λ2) and excludes the first wavelength (λ1)).

The sensor according to some example embodiments may include two infrared light sensors respectively performing separately functions and thus may work as a combination sensor. In addition, two sensors performing separately functions are stacked in each pixel, and thus the number of pixel performing functioning of each sensor is twice increased while maintaining a size and resultantly, sensitivity may be much improved.

As noted above with reference to FIG. 1, the active layer 30, or any portion of the photoelectric device 100 and/or 200, may include the compound according to some example embodiments and thus may have improved sensitivity to and/or absorbance of near-infrared light, such that the operational performance and/or efficiency of the organic sensor 400 in absorbing and/or photoelectrically converting incident near-infrared light into electrical signals (e.g., photoelectric conversion performance and/or efficiency) may be improved. In some example embodiments, the second infrared light sensor 120 may include the compound according to some example embodiments and thus may have improved sensitivity to and/or absorbance of near-infrared light, such that the operational performance and/or efficiency of the organic sensor 400 in absorbing and/or converting incident near-infrared light into electrical signals (e.g., photoelectric conversion performance and/or efficiency) may be improved. As a result, the functionality of the organic sensor 400 with regard to sensing (e.g., absorbing and/or photoelectrically converting incident light in the near-infrared wavelength region) may be improved based on including the compound in the active layer 30.

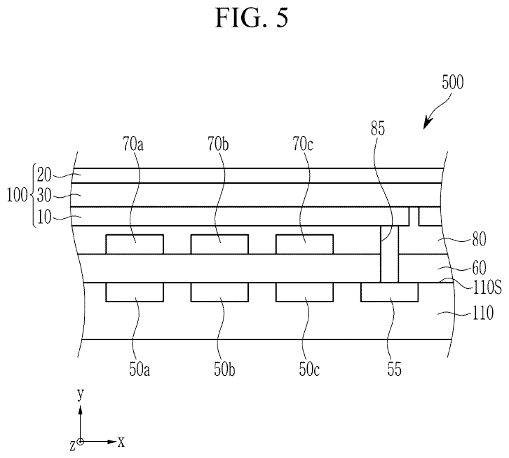

FIG. 5 is a cross-sectional view showing an example of an organic sensor according to some example embodiments.

An organic sensor according to some example embodiments may be an organic CMOS image sensor.

Referring to FIG. 5, an organic sensor 500 according to some example embodiments includes a semiconductor substrate 110 integrated with photo-sensing devices (e.g., photodiodes, including silicon-based photodiodes) 50a, 50b, and 50c, a transmission transistor (not shown), and a charge storage 55, a lower insulation layer 60, color filters 70a, 70b, and 70c, an insulation layer 80 (also referred to herein as an upper insulation layer), and a photoelectric device 100.

The semiconductor substrate 110 may be integrated with photo-sensing devices 50a, 50b, and 50c, such that the photo-sensing devices 50a, 50b, and 50c are at least partially embedded within the semiconductor substrate 110 and are vertically overlapped by the photoelectric device 100 in the vertical direction that is perpendicular to the upper surface 110S, a transmission transistor (not shown), and a charge storage 55. The photo-sensing devices 50a, 50b, and 50c may be photodiodes (e.g., silicon-based photodiodes) that may be configured to sense (e.g., selectively absorb and/or convert (e.g., photoelectrically convert)) light in different visible wavelength regions.

The photo-sensing devices 50a, 50b, and 50c, the transmission transistor, and/or the charge storage 55 may be integrated in each pixel. For example, the blue photo-sensing device 50a may be included in a blue pixel, the red photo-sensing device 50b may be included in a red pixel, and the green photo-sensing device 50c may be included in a green pixel.

The photo-sensing devices 50a, 50b, and 50c sense (e.g., selectively absorb and/or convert (into electrical signals, e.g., photoelectrically convert)) incident light, the information sensed by the photo-sensing devices may be transferred by the transmission transistor, the charge storage 55 is electrically connected to the photoelectric device 100, and the information of the charge storage 55 may be transferred by the transmission transistor.

A metal wire (not shown) and a pad (not shown) are formed on the semiconductor substrate 110. In order to decrease signal delay, the metal wire and pad may be made of a metal having low resistivity, for example, aluminum (Al), copper (Cu), silver (Ag), and alloys thereof, but are not limited thereto. However, the present inventive concepts are not limited to the above structure, and metal wire and pad may be disposed under the photo-sensing devices 50a, 50b, and 50c.

The lower insulation layer 60 is formed on the metal wire and the pad. The lower insulation layer 60 may include a same or different material composition as the insulation layer 80. Color filters 70a, 70b, and 70c are formed on the lower insulation layer 60. The color filters 70a, 70b, and 70c includes a blue filter 70a formed in a blue pixel, a red filter 70b formed in a red pixel, and a green filter 70c formed in a green pixel.

The insulation layer 80 (also referred to as an upper insulation layer) is formed on the color filters 70a, 70b, and 70c. The insulation layer 80 eliminates steps caused by the color filters 70a, 70b, and 70c and planarizes the surface.

The aforementioned photoelectric device 100 is formed on the insulation layer 80. As described above, the photoelectric device 100 includes a first electrode 10, an active layer 30, and a second electrode 20. Even though a structure in which the first electrode 10, the active layer 30 and the second electrode 20 are sequentially stacked is shown as an example in the drawing, the present inventive concepts are not limited to this structure, and the second electrode 20, the active layer 30, and the first electrode 10 may be arranged in this order.

The aforementioned photoelectric device 100 is formed on the insulation layer 80. As described above, the photoelectric device 100 includes a first electrode 10, an active layer 30, and a second electrode 20.

The first electrode 10 and the second electrode 20 may both be transparent electrodes, and the active layer 30 is the same as described above. The active layer 30 may selectively absorb and/or convert (into electrical signals, e.g., photoelectrically convert) light in a near-infrared wavelength region. As noted above with regard to photoelectric devices 100 and 200, any portion of the photoelectric device 100 (e.g., first electrode 10, second electrode 20, and/or active layer 30) may include the compound according to some example embodiments.

Incident light from the side of the second electrode 20 may be photoelectrically converted by mainly absorbing light in a near-infrared wavelength region in the active layer 30. Light in the remaining wavelength region may pass through the first electrode 10 and the color filters 70a, 70b, and 70c, the light in a blue wavelength region passing through the blue filter 70a may be sensed by the blue photo-sensing device 50a, the light in a red wavelength region passing through the red filter 70b may be sensed by the red photo-sensing device 50b, and the light in a green wavelength region passing through the green filter 70c may be sensed by the green photo-sensing device 50c.

As noted above with reference to FIG. 1, the active layer 30 may include the compound according to some example embodiments and thus may have improved sensitivity to near-infrared light, such that the operational performance and/or efficiency (e.g., photoelectric conversion performance and/or efficiency) of the organic sensor 500 in absorbing and/or converting incident near-infrared light (into electrical signals) may be improved. As a result, the functionality of the organic sensor 500 with regard to sensing (e.g., absorbing and/or photoelectrically converting incident light in the near-infrared wavelength region) may be improved based on including the compound in the active layer 30.

Accordingly, where an organic sensor includes a photoelectric device that includes the near-infrared absorber and is configured to selectively absorb and/or convert (e.g., photoelectrically convert) light in a first near-infrared wavelength region, the organic sensor may include an additional sensor that includes a plurality of photodiodes (e.g., photo-sensing devices 50a, 50b, 50c) at least partially embedded within the semiconductor substrate and configured to selectively absorb and/or convert (into electrical signals, e.g., photoelectrically convert) light in separate visible wavelength regions (e.g., blue, red, and/or green light).

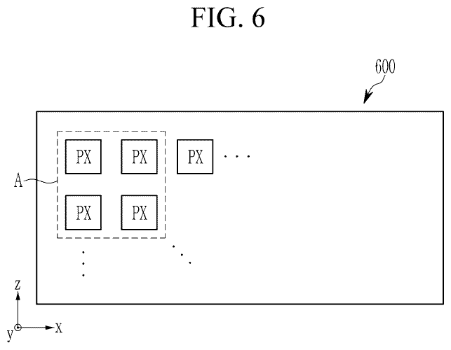

FIG. 6 is a schematic view showing an example of a pixel array of an organic sensor according to some example embodiments.

Referring to FIG. 6, an organic sensor 600 according to some example embodiments includes a plurality of pixels (PX) and the plurality of pixels (PX) may have a matrix array repeatedly arranged along rows and columns. The plurality of pixels (PX) may form (“at least partially include”) a unit pixel group (A) of for example a 2×2 array of pixels as shown in FIG. 6. However, an arrangement of the pixels are not limited thereto but variously modified, and the unit pixel group (A) may be variously modified into different arrays of pixels, including a 3×3 array, a 4×4 array, or the like, besides the 2×2 array.

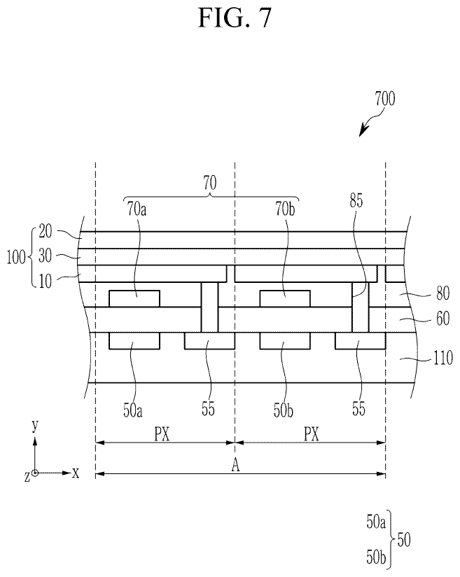

At least a part of the pixels may include a plurality of sensors having different functions inside one pixel, and the plurality of sensors may be stacked therein. In some example embodiments, each pixel (PX) may include two or more organic sensors that are configured to sense (e.g., absorb) light in different wavelength regions (“wavelength spectra of light”) in relation to each other. The organic sensors configured to sense the light in different wavelength regions each other may be stacked in a direction that is perpendicular (e.g., perpendicular within manufacturing tolerances and/or material tolerances) to an upper surface 110S of a substrate of the organic sensor 600, as shown in at least FIG. 7 (e.g., a y direction). Herein, the light of the different wavelength regions may be respectively selected from a visible wavelength region; an infrared wavelength region including a near-infrared wavelength region; and an ultraviolet (UV) wavelength region.

It will be understood that any of the organic sensors according to any of the example embodiments herein may have the pixel array structure of organic sensor 600 as shown in FIG. 6.

FIG. 7 is a cross-sectional view showing an organic sensor according to some example embodiments.

Referring to FIG. 7, an organic sensor 700 according to some example embodiments includes a semiconductor substrate 110 integrated with a visible light sensor 50 that includes photo-sensing devices 50a and 50b, a transmission transistor (not shown), and a charge storage 55; a lower insulation layer 60; a color filter layer 70; an insulation layer 80 (also referred to as an upper insulation layer when present with the lower insulation layer 60 in a same organic sensor); and a photoelectric device 100.

The semiconductor substrate 110 may be a silicon substrate, and is integrated with the photo-sensing devices 50a and 50b, the transmission transistor (not shown), and the charge storage 55. The photo-sensing devices 50a and 50b may be photodiodes (e.g., silicon-based photodiodes).

The photo-sensing devices 50a and 50b may sense light, the information sensed by the photo-sensing devices may be transferred by the transmission transistor, the charge storage 55 is electrically connected to the photoelectric device 100, and the information of the charge storage 55 may be transferred by the transmission transistor.

A metal wire (not shown) and a pad (not shown) are formed on the semiconductor substrate 110. In order to decrease signal delay, the metal wire and pad may be made of a metal having low resistivity, for example, aluminum (Al), copper (Cu), silver (Ag), and alloys thereof, but is not limited thereto. However, the inventive concepts are not limited to such structure, and the metal wire and pad may be disposed under the photo-sensing devices 50a and 50b.

The lower insulation layer 60 is formed on the metal wire and the pad. The lower insulation layer 60 may be made of an inorganic insulating material such as a silicon oxide and/or a silicon nitride, or a low dielectric constant (low K) material such as SiC, SiCOH, SiCO, and SiOF. The lower insulation layer 60 has a trench 85 exposing the charge storage 55. The trench 85 may be filled with fillers.

A color filter layer 70 is formed on the lower insulation layer 60. The color filter layer 70 includes a blue filter 70a formed in the blue pixel and a red filter 70b formed in the red pixel. In FIG. 7, a green filter is not included, but a green filter may be further included.

The insulation layer 80 is formed on the color filter layer 70. The insulation layer 80 eliminates a step caused by the color filter layer 70 and smoothens the surface. The insulation layer 80 and lower insulation layer 60 may include a contact hole (not shown) exposing a pad, and a through hole (e.g., trench 85) exposing the charge storage 55 of a pixel (e.g., a green pixel).

The aforementioned photoelectric device 100 is formed on the insulation layer 80. As described above, the photoelectric device 100 includes a first electrode 10, an active layer 30, and a second electrode 20. The photoelectric device 100 may be the same as the photoelectric device 100 of FIG. 1. In some example embodiments, the photoelectric device 100 of FIG. 7 may be replaced with the photoelectric device 200 of FIG. 2.

The first electrode 10 and the second electrode 20 may be all light-transmitting electrodes and the active layer 30 may selectively absorb and/or convert (into electrical signals, e.g., photoelectrically convert) light in a near-infrared wavelength region. In some example embodiments, including the example embodiments shown in FIG. 7, the active layer 30 may additionally selectively absorb and/or convert (into electrical signals, e.g., photoelectrically convert) light in a visible wavelength region (e.g., green light).

Focusing lens (not shown) may be further formed on the photoelectric device 100. The focusing lens may control a direction of incident light and gather the light in one region. The focusing lens may have a shape of, for example, a cylinder or a hemisphere, but is not limited thereto.

In FIG. 7, a structure where the photoelectric device 100 selectively absorbing light in a near-infrared wavelength region is stacked on the semiconductor substrate 110 is illustrated, but the present inventive concepts are not limited thereto. Among the light incident on the organic sensor 700 at an upper surface of the photoelectric device 100, at least light in a near-infrared wavelength region may be mainly absorbed in the active layer 30 and photoelectrically converted, and light in a visible (e.g., blue, green, and/or red) wavelength region may pass through the first electrode 10 and be sensed by the photo-sensing devices 50a and 50b.

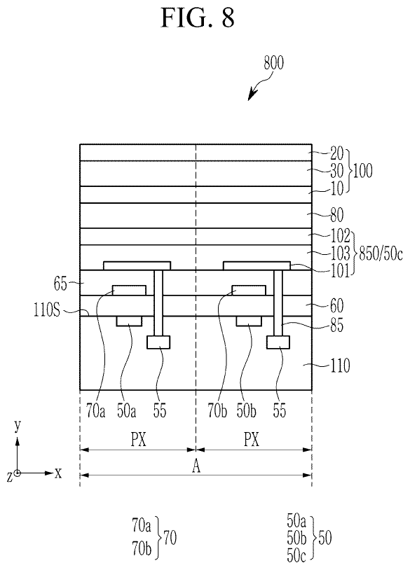

FIG. 8 is a cross-sectional view showing an organic sensor according to some example embodiments.

Referring to FIG. 8, the organic sensor 800 according to some example embodiments includes a visible light sensor 50, and the photoelectric device 100 as described above.

Referring to FIG. 8, in the organic sensor 800 according to some example embodiments, the visible light sensor 50 may be a combination of a photodiode integrated in the semiconductor substrate 110 and a photoelectric device disposed on the semiconductor substrate 110, and the photoelectric device 100 may be a separate photoelectric device.

Accordingly, where an organic sensor includes a photoelectric device (e.g., 100) that includes the near-infrared absorber and is configured to selectively absorb and/or convert (e.g., photoelectrically convert) light in a first near-infrared wavelength region, and an additional sensor (e.g., 50a and/or 50b) configured to selectively absorb and/or convert (e.g., photoelectrically convert) light in a separate wavelength region of incident light, the organic sensor may further include an additional photoelectric device (e.g., 50c) on the semiconductor substrate, the additional photoelectric device being between the photoelectric device 100 and the semiconductor substrate 110, the additional photoelectric device configured to selectively absorb and/or convert (e.g., photoelectrically convert) light in an additional wavelength region of incident light that is different from the first near-infrared wavelength region and different from the separate wavelength region(s) absorbed by the additional sensor (e.g., 50a and/or 50b).

In the semiconductor substrate 110, the blue photo-sensing device 50a, the red photo-sensing device 50b, the charge storage 55, and a transmission transistor (not shown) are integrated. The blue photo-sensing device 50a and the red photo-sensing device 50b are photodiodes (e.g., silicon-based photodiodes) and spaced apart from each other in a horizontal direction of the semiconductor substrate 110. The blue photo-sensing device 50a is integrated in a blue pixel, and the red photo-sensing device 50b is integrated in a red pixel.

On the semiconductor substrate 110, the lower insulation layer 60 and the color filter layer 70 are formed. The color filter layer 70 includes a blue filter 70a overlapped with the blue photo-sensing device 50a and a red filter 70b overlapped with the red photo-sensing device 50b.

An intermediate insulation layer 65 is formed on the color filter layer 70. The lower insulation layer 60 and the intermediate insulation layer 65 may have a through hole (e.g., trench 85) exposing the charge storage 55. The through hole (e.g., trench 85) may be filled with fillers. At least one of the lower insulation layer 60 or intermediate insulation layer 65 may be omitted.

On the intermediate insulation layer 65, the additional photoelectric device 850 is formed. In the example embodiments shown in FIG. 8, the additional photoelectric device 850 is also the green photo-sensing device 50c, but it will be understood that in some example embodiments the additional photoelectric device 850 may be configured to sense (e.g., selectively absorb and/or convert (into electrical signals, e.g., photoelectrically convert)) light in a wavelength region that is different from the green wavelength region and may be a non-visible wavelength region (e.g., a second near-infrared wavelength region) that is different from the first near-infrared wavelength region sensed by the photoelectric device 100. The additional photoelectric device 850 includes a first electrode 101 (lower electrode) and a second electrode 102 (upper electrode) facing each other, and an active layer 103 between the first electrode 101 and the second electrode 102. One of the first electrode 101 or the second electrode 102 is an anode and the other is a cathode.

Both of the first electrode 101 and the second electrode 102 may be a light-transmitting electrode, and the light-transmitting electrode may be made of, in some example embodiments, a transparent conductor such as indium tin oxide (ITO) or indium zinc oxide (IZO), or may be a metal thin layer having a thin thickness of several nanometers to several tens of nanometers or a metal thin layer having a thin thickness of several nanometers to several tens of nanometers doped with a metal oxide.

The active layer 103 may have a composition similar to that of the active layer 30 of photoelectric device 100 and/or 200, and may include the near-infrared absorber. The active layer 103 may be a photoelectric conversion layer configured to selectively absorb and/or convert (into electrical signals) (e.g., photoelectrically convert) light in at least a portion of a wavelength region (e.g., wavelength spectrum of the light). The active layer 103 may for example convert at least a portion of light in a green wavelength region (hereinafter, referred to as “green light”), light in a blue wavelength region (hereinafter, referred to as “blue light”), light in a red wavelength region (hereinafter, referred to as “red light”), light in an infrared wavelength region (hereinafter, referred to as “infrared light”), light in an ultraviolet wavelength region (hereinafter, referred to as “ultraviolet light”), or any combination thereof, or the like, into an electrical signal.

For example, the active layer 103 may be configured to selectively absorb and/or convert (into electrical signals, e.g., photoelectrically convert) at least one of the green light, the blue light, the red light, the infrared light, or the ultraviolet light. Herein, the selective absorption of at least one from the green light, the blue light, the red light, the infrared light, or the ultraviolet light means that a light-absorption spectrum has a peak absorption wavelength (λₘₐₓ) in one of about 500 nm to about 600 nm, greater than or equal to about 380 nm and less than about 500 nm, greater than about 600 nm and less than or equal to about 700 nm, or greater than about 700 nm and less than or equal to about 3000 nm and a light-absorption spectrum in the corresponding wavelength region is remarkably higher than those in the other wavelength regions.

The active layer 103 may include at least one p-type semiconductor and at least one n-type semiconductor which form a pn junction and may produce excitons by receiving light from outside and then separate the produced excitons into holes and electrons. The p-type semiconductor and the n-type semiconductor may be independently light-absorbing materials, and for example at least one of the p-type semiconductor or the n-type semiconductor may be an organic light-absorbing material. For example, at least one of the p-type semiconductor or the n-type semiconductor may be a wavelength-selective light-absorbing material that selectively absorbs light in a particular (or, predetermined) wavelength region, and for example at least one of the p-type semiconductor or the n-type semiconductor may be a wavelength-selective organic light-absorbing material. The p-type semiconductor and the n-type semiconductor may have a peak absorption wavelength (λₘₐₓ) in the same wavelength region or in a different wavelength region, among a green wavelength region, a blue wavelength region, a red wavelength region, and an infrared wavelength region. For example, the p-type semiconductor may be an organic material having a core structure including an electron-donor moiety, a pi conjugation linking group, and an electron-acceptor moiety. The p-type semiconductor may be for example represented by Chemical Formula 4, but is not limited thereto.

EDG-HA-EAG [Chemical Formula 4]

In Chemical Formula 4,

HA may be a C2 to C30 heterocyclic group having at least one of S, Se, Te, or Si, EDG may be an electron-donating group, and EAG may be an electron accepting group. For example, the p-type semiconductor represented by Chemical Formula 4 may be for example represented by Chemical Formula 4A.

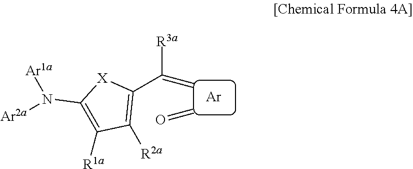

In Chemical Formula 4A,

X may be S, Se, Te, SO, SO₂, or SiR^{a}R^{b}, Ar may be a substituted or unsubstituted C6 to C30 arylene group, a substituted or unsubstituted C3 to C30 heterocyclic group, or a fused ring of the foregoing two or more, Ar^{1a} and Ar^{2a} may each independently be a substituted or unsubstituted C6 to C30 aryl group or a substituted or unsubstituted C3 to C30 heteroaryl group, Ar^{1a} and Ar^{2a} may each independently be present alone or may be linked with each other to form a fused ring, and R^{1a} to R^{3a}, R^{a}, and R^{b} may each independently be hydrogen, deuterium, a substituted or unsubstituted C1 to C30 alkyl group, a substituted or unsubstituted C6 to C30 aryl group, a substituted or unsubstituted C3 to C30 heteroaryl group, a substituted or unsubstituted C1 to C6 alkoxy group, a halogen, or a cyano group.

For example, in Chemical Formula 4A, Ar^{1a} and Ar^{2a} may each independently be a substituted or unsubstituted phenyl group, a substituted or unsubstituted naphthyl group, a substituted or unsubstituted anthracenyl group, a substituted or unsubstituted phenanthrenyl group, a substituted or unsubstituted pyridinyl group, a substituted or unsubstituted pyridazinyl group, a substituted or unsubstituted pyrimidinyl group, a substituted or unsubstituted pyrazinyl group, a substituted or unsubstituted quinolinyl group, a substituted or unsubstituted isoquinolinyl group, a substituted or unsubstituted naphthyridinyl group, a substituted or unsubstituted cinnolinyl group, a substituted or unsubstituted quinazolinyl group, a substituted or unsubstituted phthalazinyl group, a substituted or unsubstituted benzotriazinyl group, a substituted or unsubstituted pyridopyrazinyl group, a substituted or unsubstituted pyridopyrimidinyl group, or a substituted or unsubstituted pyridopyridazinyl group. For example, Ar^{1a} and Ar^{2a} of Chemical Formula 4A may be linked with each other to form a ring or for example, Ar^{1a} and Ar^{2a} may be linked with each other by one of a single bond, —(CR^{g}R^{h})ₙ₂— (n2 is 1 or 2), —O—, —S—, —Se—, —N═, —NRⁱ—, —SiR^{j}R^{k}—, or —GeR^{l}R^{m}— to form a ring. Herein, R^{g} to R^{m} may each independently be hydrogen, a substituted or unsubstituted C1 to C30 alkyl group, a substituted or unsubstituted C6 to C30 aryl group, a substituted or unsubstituted C3 to C30 heteroaryl group, a substituted or unsubstituted C1 to C6 alkoxy group, a halogen, or a cyano group.

For example, the p-type semiconductor represented by Chemical Formula 4 may be for example represented by Chemical Formula 4B.

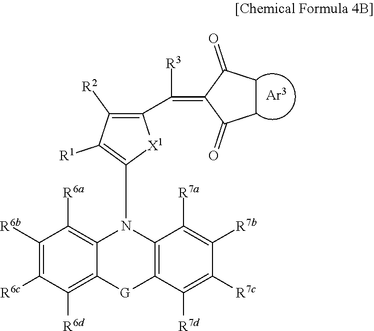

In Chemical Formula 4B3,

X¹ may be Se, Te, O, S, SO, or SO₂, Ar³ may be a substituted or unsubstituted C6 to C30 arylene group, a substituted or unsubstituted C3 to C30 heterocyclic group, or a fused ring of the foregoing two or more, R¹ to R³ may each independently be one of hydrogen, deuterium, a substituted or unsubstituted C1 to C30 alkyl group, a substituted or unsubstituted C1 to C30 alkoxy group, a substituted or unsubstituted C6 to C30 aryl group, a substituted or unsubstituted C3 to C30 heteroaryl group, a halogen, a cyano group, a cyano-containing group, or any combination thereof, G may be one of a single bond, —O—, —S—, —Se—, —N═, —(CR^{f}R^{g})ₖ—, —NR^{h}—, —SiRⁱR^{j}—, —GeR^{k}R^{l}—, —(C(R^{m})═C(Rⁿ))—, or SnR^{o}R^{p}, wherein R^{f}, R^{g}, R^{h}, Rⁱ, R^{j}, R^{k}, R^{l}, R^{m}, Rⁿ, R^{o}, and R^{p} may each independently be one of hydrogen, a halogen, a substituted or unsubstituted C1 to C10 alkyl group, a substituted or unsubstituted C1 to C10 alkoxy group, and a substituted or unsubstituted C6 to C12 aryl group, R^{f} and R^{g}, Rⁱ and R^{j}, R^{k} and R^{l}, R^{m} and Rⁿ, and R^{o} and R^{p} may each independently be present alone or may be linked with each other to provide a ring, and k may be 1 or 2, R^{6a} to R^{6d} and R^{7a} to R^{7d} may each independently be one of hydrogen, a substituted or unsubstituted C1 to C30 alkyl group, a substituted or unsubstituted C6 to C30 aryl group, a substituted or unsubstituted C3 to C30 heteroaryl group, a halogen, a cyano group, a cyano-containing group, or any combination thereof, R^{6a} to R^{6d} may each independently be present alone or adjacent two thereof may be linked with each other to form a fused ring, and R^{7a} to R^{7d} may each independently be present alone or adjacent two thereof may be linked with each other to form a fused ring.

For example, Ar³ of Chemical Formula 4B may be a benzene ring, a naphthylene ring, an anthracene ring, a thiophene ring, a selenophene ring, a tellurophene ring, a pyridine ring, a pyrimidine ring, or a fused ring of the foregoing two or more. The n-type semiconductor may be for example fullerene or a fullerene derivative, but is not limited thereto.

The active layer 103 may be an intrinsic layer (an I layer) wherein the p-type semiconductor and the n-type semiconductor are blended as a bulk heterojunction. Herein, the p-type semiconductor and the n-type semiconductor may be blended in a volume ratio of about 1:9 to about 9:1, for example about 2:8 to about 8:2, about 3:7 to about 7:3, about 4:6 to about 6:4, or about 5:5. The active layer 103 may include a bi-layer including a p-type layer including the aforementioned p-type semiconductor and an n-type layer including the aforementioned n-type semiconductor.

Herein, a thickness ratio of the p-type layer and the n-type layer may be about 1:9 to about 9:1, for example about 2:8 to about 8:2, about 3:7 to about 7:3, about 4:6 to about 6:4, or about 5:5. The active layer 103 may further include a p-type layer and/or an n-type layer in addition to the intrinsic layer. The p-type layer may include the aforementioned p-type semiconductor (e.g., the aforementioned compound according to some example embodiments) and the n-type layer may include the aforementioned n-type semiconductor. For example, the active layer 103 may include the aforementioned p-type semiconductor and the aforementioned n-type semiconductor in various combinations of p-type layer/I layer, I layer/n-type layer, p-type layer/I layer/n-type layer, and the like.

In the example embodiments shown in FIG. 8, the active layer 103 is configured to selectively absorb and/or convert (into electrical signals, e.g., photoelectrically convert) green light, but example embodiments are not limited thereto, and in some example embodiments the active layer 103 may selectively absorb and/or convert (into electrical signals) (e.g., photoelectrically convert) blue light, red light, or any visible wavelength region of light, or any non-visible wavelength region of light (e.g., a second wavelength region of near-infrared light that is selectively transmitted by the photoelectric device 100).

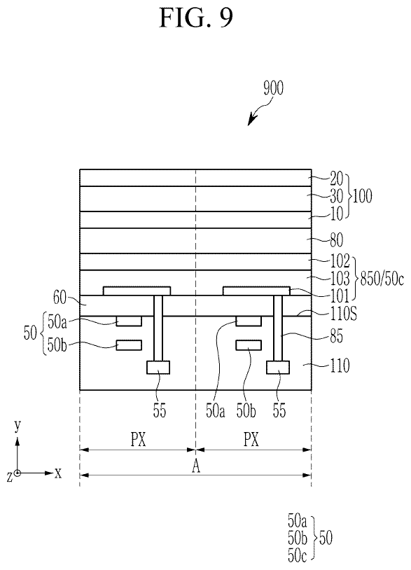

FIG. 9 is a cross-sectional view showing an organic sensor according to some example embodiments.

Referring to FIG. 9, the organic sensor 900 according to some example embodiments includes the visible light sensor 50, and the photoelectric device 100 like that of some example embodiments. The visible light sensor 50 includes the blue photo-sensing device 50a and the red photo-sensing device 50b integrated in the semiconductor substrate 110 and an additional photoelectric device 850 that includes a green photo-sensing device 50c disposed on the semiconductor substrate 110, wherein the blue photo-sensing device 50a and the red photo-sensing device 50b may be photodiodes (e.g., silicon-based photodiodes), and the additional photoelectric device 850 may be a green photo-sensing device 50c that may be the same as, or different than, the green photo-sensing device 50c shown in FIG. 8. The additional photoelectric device 850 includes a first electrode 101, active layer 103, and a second electrode (upper electrode) 102, and the photoelectric device 100 includes a first electrode 10, an active layer 30, and a second electrode 20.

However, in the organic sensor 900 according to some example embodiments, the blue photo-sensing device 50a and the red photo-sensing device 50b integrated in the semiconductor substrate 110 are stacked in a vertical direction (e.g., perpendicular to the upper surface 110S of the semiconductor substrate 110). The blue photo-sensing device 50a and the red photo-sensing device 50b may be configured to selectively absorb and/or convert (e.g., photoelectrically convert) light in each wavelength region depending on a stacking depth and thus sense it. In other words, the red photo-sensing device 50b configured to selectively absorb and/or convert (into electrical signals) (e.g., photoelectrically convert) red light in a long wavelength region is disposed deeper from the surface of the semiconductor substrate 110 than the blue photo-sensing device 50a configured to selectively absorb and/or convert (into electrical signals) (e.g., photoelectrically convert) blue light in a short wavelength region. In this way, the color filter layer 70 may be omitted by separating absorption wavelengths depending on the stacking depth.

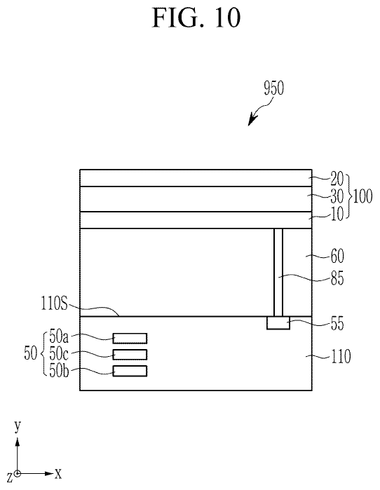

FIG. 10 is a cross-sectional view showing an organic sensor according to some example embodiments.

Referring to FIG. 10, the organic sensor 950 according to some example embodiments includes the visible light sensor 50, and the photoelectric device 100 like that of some example embodiments. The visible light sensor 50 includes the blue photo-sensing device 50a, the green photo-sensing device 50c, and the red photo-sensing device 50b integrated in the semiconductor substrate 110, wherein the blue photo-sensing device 50a, the green photo-sensing device 50c, and the red photo-sensing device 50b may be photodiodes. In the organic sensor 950 according to some example embodiments, the blue photo-sensing device 50a, the green photo-sensing device 50c, and the red photo-sensing device 50b integrated in the semiconductor substrate 110 are stacked in a vertical direction. The blue photo-sensing device 50a, the green photo-sensing device 50c, and the red photo-sensing device 50b may be configured to selectively absorb and/or convert (into electrical signals, e.g., photoelectrically convert) light in each wavelength region depending on a stacking depth from the upper surface 110S and thus sense it. In other words, the red photo-sensing device 50b configured to selectively absorb and/or convert (into electrical signals, e.g., photoelectrically convert) red light in a long wavelength region is disposed deeper from the upper surface 110S of the semiconductor substrate 110 than the blue photo-sensing device 50a configured to selectively absorb and/or convert (into electrical signals) (e.g., photoelectrically convert) blue light in a short wavelength region, and the green photo-sensing device 50c configured to selectively absorb and/or convert (into electrical signals, e.g., photoelectrically convert) green light in a medium wavelength region is disposed deeper from the upper surface 110S of the semiconductor substrate 110 than the blue photo-sensing device 50a and closer to the upper surface 110S of the semiconductor substrate 110 than the red photo-sensing device 50b. In this way, the color filter layer 70 may be omitted by separating absorption wavelengths depending on the stacking depth.

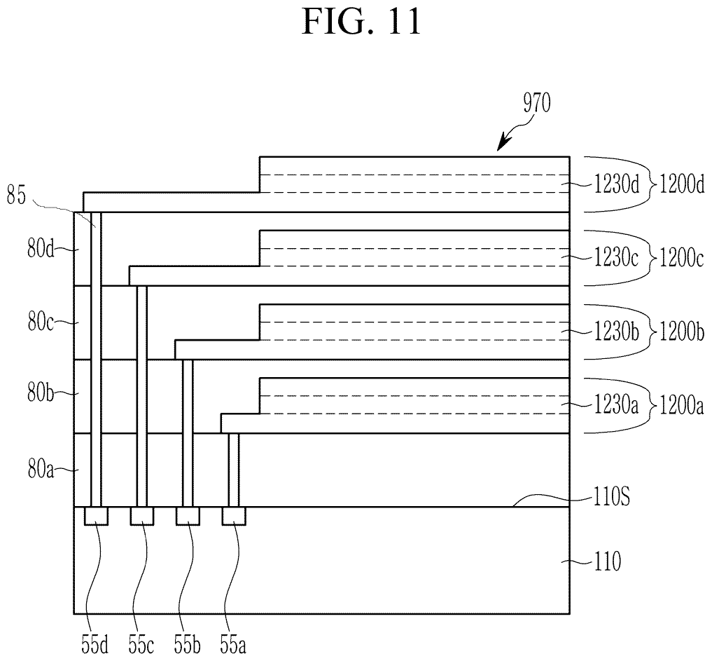

FIG. 11 is a cross-sectional view showing an organic sensor according to some example embodiments.

Referring to FIG. 11, the organic sensor 970 according to some example embodiments includes a first photoelectric device (e.g., infrared/near-infrared photoelectric device 1200d) configured to selectively absorb and/or convert (e.g., photoelectrically convert) light in an infrared/near-infrared wavelength spectrum of incident light (e.g., a first near-infrared wavelength region), and at least one additional photoelectric device (e.g., 1200a to 1200c) vertically stacked between the first photoelectric device and a semiconductor substrate 110, each separate photoelectric device of the at least one additional photoelectric device including a separate photoelectric conversion layer and configured to selectively absorb and/or convert (e.g., photoelectrically convert) light in a separate (e.g., respective) wavelength region of incident light that is different from the first near-infrared wavelength region and which may be a separate visible and/or non-visible wavelength region. For example, as shown in FIG. 11, the organic sensor 970 may include additional photoelectric devices that include a red photoelectric device configured to selectively absorb and/or convert (into electrical signals) (e.g., photoelectrically convert) light in a red wavelength spectrum of incident light, a green photoelectric device configured to selectively absorb and/or convert (into electrical signals) light in a green wavelength spectrum of incident light, and a blue photoelectric device configured to selectively absorb and/or convert (into electrical signals) light in a blue wavelength spectrum of incident light, and they are stacked in the vertical direction (e.g., z-direction, a direction extending perpendicular to an upper surface 110S of the semiconductor substrate 110, etc.).

Accordingly, it will be understood that, as shown in FIG. 11, the organic sensor 970 may include a plurality of photoelectric devices 1200a to 1200d that are stacked vertically on the semiconductor substrate 110, such that the plurality of photoelectric devices 1200a to 1200d overlap each other in a direction extending perpendicular to an upper surface 110S of the semiconductor substrate 110. While the organic sensor 970 includes multiple additional photoelectric devices 1200a-1200c in addition to the first photoelectric device (e.g., fourth photoelectric device 1200d) configured to selectively absorb and/or convert light in the first near-infrared wavelength region, it will be understood that in some example embodiments the organic sensor 970 may be limited to a single additional photoelectric device (e.g., any of 1200a to 1200c) between the photoelectric device 1200d and the semiconductor substrate 110.

The organic sensor 970 according to some example embodiments includes a semiconductor substrate 110, a lower insulation layer 80a, an intermediate insulation layer 80b, another intermediate insulation layer 80c, an upper insulation layer 80d, a first photoelectric device 1200a, a second photoelectric device 1200b, a third photoelectric device 1200c, and a fourth photoelectric device 1200d. In some example embodiments, the fourth photoelectric device 1200d may be referred to as a first photoelectric device configured to selectively absorb and/or convert (e.g., photoelectrically convert) light in a first near-infrared wavelength region, and the first to third photoelectric devices 1200a to 1200c may be collectively referred to as at least one additional photoelectric device configured to selectively absorb and/or convert (e.g., photoelectrically convert) light in one or more separate wavelength regions different from the first near-infrared wavelength region. As shown, the first to fourth photoelectric devices 1200a to 1200d are stacked vertically on the semiconductor substrate 110, such that the first to fourth photoelectric devices 1200a to 1200d overlap each other in a direction extending perpendicular to an upper surface 110S of the semiconductor substrate 110. The semiconductor substrate 110 may be a silicon substrate, and is integrated with the transmission transistor (not shown) and charge storages.

The first through third photoelectric devices 1200a to 1200c may have a same structure as the additional photoelectric devices 850 shown in FIGS. 8 and 9. However, each separate photoelectric device 1200a to 1200c may be configured to photoelectrically convert a separate wavelength region of visible and/or non-visible (e.g., near-infrared) light, and the photoelectric conversion layers 1230a to 1230c may have the same structure and/or composition as various example embodiments (e.g., different example embodiments) of the active layer 103 and/or active layer 30 as described herein so as to be configured to selectively absorb and/or convert (into electrical signals) (e.g., photoelectrically convert) different visible and/or non-visible wavelength regions of light, and may include the aforementioned compound according to some example embodiments.

The first photoelectric device 1200a is formed on the lower insulation layer 80a. The first photoelectric device 1200a includes a photoelectric conversion layer 1230a. The first photoelectric device 1200a may be any one of the photoelectric devices described herein according to any of the example embodiments. The photoelectric conversion layer 1230a may selectively absorb and/or convert (into electrical signals) (e.g., photoelectrically convert) light in one of infrared, red, blue, or green wavelength spectra of incident light. For example, the first photoelectric device 1200a may be a blue photoelectric device.

The intermediate insulation layer 80b is formed on the first photoelectric device 1200a.

The second photoelectric device 1200b is formed on the intermediate insulation layer 80b. The second photoelectric device 1200b includes a photoelectric conversion layer 1230b. The second photoelectric device 1200b may be any one of the photoelectric devices described herein according to any of the example embodiments. The photoelectric conversion layer 1230b may selectively absorb and/or convert (into electrical signals) (e.g., photoelectrically convert) light in one of infrared, red, blue, or green wavelength spectra of incident light. For example, the second photoelectric device 1200b may be a green photoelectric device. Another intermediate insulation layer 80c is formed on the second photoelectric device 1200b.

The third photoelectric device 1200c is formed on the intermediate insulation layer 80c. The third photoelectric device 1200c includes a photoelectric conversion layer 1230c. The third photoelectric device 1200c any one of the photoelectric devices described herein according to any of the example embodiments. The photoelectric conversion layer 1230c may selectively absorb and/or convert (into electrical signals) (e.g., photoelectrically convert) light in one of infrared, red, blue, or green wavelength spectra of incident light. For example, the third photoelectric device 1200c may be a red photoelectric device.

The upper insulation layer 80d is formed on the third photoelectric device 1200c.

The lower insulation layer 80a, the intermediate insulation layers 80b and 80c, and the upper insulation layer 80d have a plurality of through holes exposing the charge storages 55a, 55b, 55c, and 55d.

The fourth photoelectric device 1200d is formed on the upper insulation layer 80d. The fourth photoelectric device 1200d includes a photoelectric conversion layer 1230d. The fourth photoelectric device 1200d may be any one of the photoelectric devices described herein according to any of the example embodiments. The photoelectric conversion layer 1230d may selectively absorb and/or convert (into electrical signals) (e.g., photoelectrically convert) light in one of infrared, red, blue, or green wavelength spectra of light. For example, the fourth photoelectric device 1200d may be an infrared/near-infrared photoelectric device that may include the near-infrared absorber. In the drawing, the first photoelectric device 1200a, the second photoelectric device 1200b, the third photoelectric device 1200c, and the fourth photoelectric device 1200d are sequentially stacked, but the present inventive concepts are not limited thereto, and they may be stacked in various orders.

As described above, the first photoelectric device 1200a, the second photoelectric device 1200b, the third photoelectric device 1200c, and the fourth photoelectric device 1200d have a stack structure, and thus the size of an organic sensor may be reduced to realize a down-sized organic sensor.

The aforementioned organic sensor may be applied to various electronic devices, for example and the electronic devices may include for example a camera, a camcorder, a mobile phone internally having them, a display device, a security device, or a medical device, but are not limited thereto.

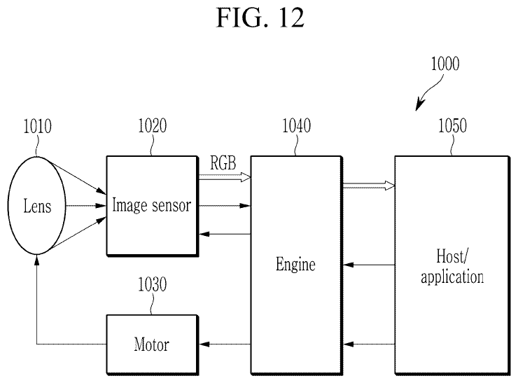

FIG. 12 is a block diagram of a digital camera including an image sensor according to some example embodiments.

Referring to FIG. 12, a digital camera 1000 includes a lens 1010, an image sensor 1020, a motor 1030, and an engine 1040. The image sensor 1020 may be one of image sensors according to embodiments shown in FIGS. 3 to 11.

The lens 1010 concentrates incident light on the image sensor 1020. The image sensor 1020 generates RGB data for received light through the lens 1010.

In some example embodiments, the image sensor 1020 may interface with engine 1040. The motor 1030 can adjust the focus of the lens 1010 or adjust the shutter in response to a control signal received from the engine 1040. The engine 1040 may control the image sensor 1020 and the motor 1030.

The engine 1040 may be connected to a host/application 1050.

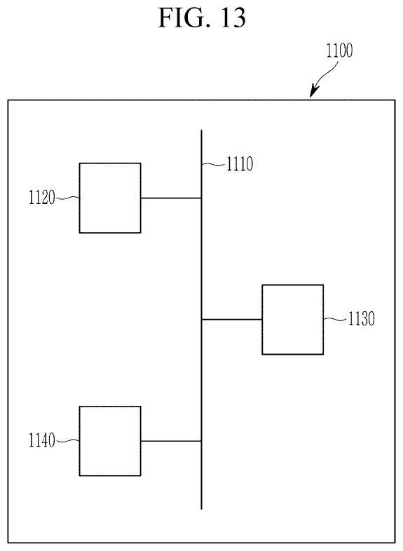

FIG. 13 is a schematic view of an electronic device according to some example embodiments.

Referring to FIG. 13, an electronic device 1100 may include a processor 1120, a memory 1130, and an image sensor 1140 that are electrically coupled together via a bus 1110.

The image sensor 1140 may be an image sensor and/or an organic sensor according to the aforementioned implementation examples. The memory 1130 may be a non-transitory computer-readable medium and may store an instruction program. The memory 1130 may be a nonvolatile memory, such as a flash memory, a phase-change random access memory (PRAM), a magneto-resistive RAM (MRAM), a resistive RAM (ReRAM), or a ferro-electric RAM (FRAM), or a volatile memory, such as a static RAM (SRAM), a dynamic RAM (DRAM), or a synchronous DRAM (SDRAM). Processor 1120 may execute stored instruction programs to perform one or more functions. For example, the processor 1120 may be configured to process electrical signals generated by the image sensor 1140. The processor 1120 may include processing circuitry, such as hardware, including logic circuitry; a hardware/software combination, such as a processor that runs the software; or any combination thereof. For example, the processing circuitry may include a central processing unit (CPU), an application processor (AP), an arithmetic logic unit (ALU), a graphic processing unit (GPU), a digital signal processor, a microcomputer, a field programmable gate array (FPGA), a System-on-Chip (SoC) a programmable logic unit, a microprocessor, or an application-specific integrated circuit (ASIC), etc. The processor 1120 may be configured to generate output (e.g., an image to be displayed on a display interface) based on this processing.

As described herein, any devices, systems, modules, portions, units, controllers, circuits, and/or portions thereof according to any of the example embodiments, and/or any portions thereof (including, without limitation, the digital camera 1000, the image sensor 1020, the motor 1030, the engine 1040, the host/application 1050, the electronic device 1100, the processor 1120, the memory 1130, the image sensor 1140, any portion thereof, or the like) may include, may be included in, and/or may be implemented by one or more instances of processing circuitry such as hardware including logic circuits; a hardware/software combination such as a processor executing software; or a combination thereof. For example, the processing circuitry more specifically may include, but is not limited to, a central processing unit (CPU), an arithmetic logic unit (ALU), a graphics processing unit (GPU), an application processor (AP), a digital signal processor (DSP), a microcomputer, a field programmable gate array (FPGA), and programmable logic unit, a microprocessor, application-specific integrated circuit (ASIC), a neural network processing unit (NPU), an Electronic Control Unit (ECU), an Image Signal Processor (ISP), and the like. In some example embodiments, the processing circuitry may include a non-transitory computer readable storage device (e.g., a memory), for example a solid state drive (SSD), storing a program of instructions, and a processor (e.g., CPU) configured to execute the program of instructions to implement the functionality and/or methods performed by some or all of any devices, systems, modules, portions, units, controllers, circuits, and/or portions thereof according to any of the example embodiments.

Hereinafter, some example embodiments are illustrated in more detail with reference to examples. However, the present scope of the inventive concepts is not limited to these examples.

### EXAMPLES

### Synthesis Example 1: Synthesis of Compound 1

Compound 1 is synthesized through the reaction process shown in Reaction Scheme 1.

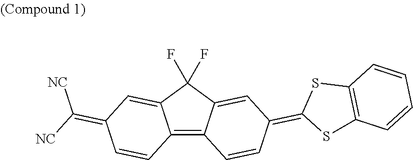 [SMILES: N#CC(C#N)=c1ccc2c(c1)C(F)(F)c1cc(=C3Sc4ccccc4S3)ccc1=2]

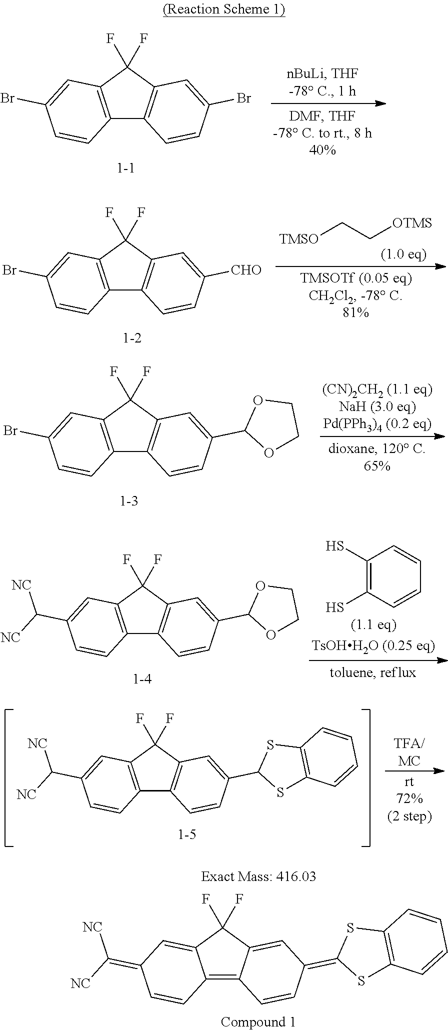

As shown in Reaction Scheme 1, Compound 1-1 (QP8094, Combi-Block Inc.) is used as a starting material, and an equivalent amount and a dropping rate of n-BuLi are controlled in a THE solvent to synthesize a product indicated by 1-2 (Compound 1-2) with a yield of about 40%, and then, acetal indicated by 1-3 (Compound 1-3) is synthesized with a yield of about 81% according to the above reaction scheme. Subsequently, Compound 1-4 is synthesized with a yield of about 65% through a coupling reaction with malononitrile with a catalyst amount of Pd(PPh₃)₄, and an acetal structure of Compound 1-4 is substituted with of benzene-1,2-dithiol to synthesize Compound 1-5. Subsequently, Compound 1-5 in the reaction mixture state is treated with TFA in two steps at room temperature (rt.) to synthesize Compound 1 with a yield of about 72%.

Hereinafter, each reaction step of Reaction Scheme 1 is described in detail.

(1) Synthesis Step of Compound 1-2 from Compound 1-1

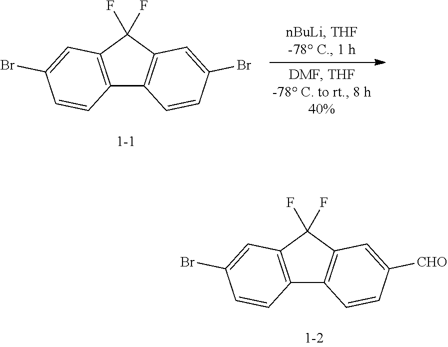 [SMILES: FC1(F)c2cc(Br)ccc2-c2ccc(Br)cc21 ; O=Cc1ccc2c(c1)C(F)(F)c1cc(Br)ccc1-2]

In a 100 ml round-bottomed flask dried with anhydrous flame, 2.04 g (5.68 mmol, 1.00 eq) of Compound 1-1 as a starting material is dissolved in THE (50 ml) and then, cooled to −78° C., and 5.43 ml (6.248 mmol, 1.10 eq) of 1.15 M n-BuLi is slowly added thereto and then, stirred at −78° C. for 1 hour. Subsequently, 0.88 ml (11.360 mmol, 2.00 eq) of DMF is added thereto and then, slowly heated to room temperature from −78° C. and stirred overnight. After checking the reaction progress by TLC (EA:Hex=1:20), the reaction is terminated by using an HCl aqueous solution. Subsequently, an organic layer is extracted therefrom with ethyl acetate and H₂O and dried with MgSO₄. Then, column chromatography (Hex to EA:Hex=1:20) is performed to obtain 527 mg (30%) of Compound 1-2 of monoaldehyde.

(2) Synthesizing Compound 1-3 from Compound 1-2

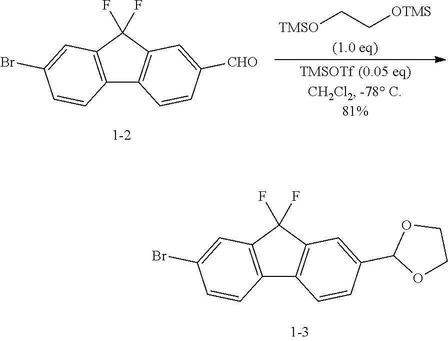

In a 100 ml round-bottomed flask dried with anhydrous flame, Compound 1-2 (205 mg, 0.6632 mmol, 1.00 eq) is dissolved in CH₂Cl₂ (3 ml) and then, cooled to −78° C., and (TMSOCH₂)₂ (0.163 ml, 0.6640 mmol, 1.00 eq), and TMSOTf (7.4 ul, 0.0332 mmol, 0.05 eq) are sequentially added thereto and then, stirred at −78° C. for 6 hours. After checking the reaction progress with TLC (EA:Hex=1:20) and then, terminating the reaction by using triethylamine, an organic layer is extracted therefrom with CH₂Cl₂ and H₂O and then, dried with Na₂SO₄. 190 mg (81%) of Compound 1-3 is obtained therefrom without further purification.

(3) Synthesizing Compound 1-4 from Compound 1-3

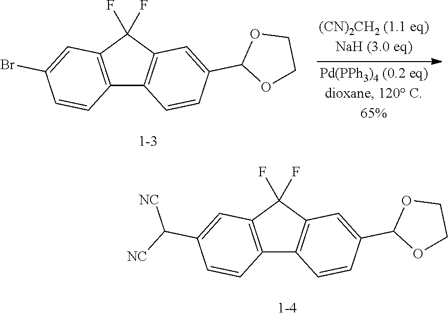 [SMILES: FC1(F)c2cc(Br)ccc2-c2ccc(C3OCCO3)cc21 ; N#CC(C#N)c1ccc2c(c1)C(F)(F)c1cc(C3OCCO3)ccc1-2]

In a 100 ml round-bottomed flask dried with anhydrous flame, (CN)₂CH₂ (46 mg, 0.6951 mmol, 1.20 eq) is dissolved in 1,4-dioxane (3 ml). After cooling to 0° C., 60% NaH (71 mg, 1.7379 mmol, 3.00 eq) is added thereto. The mixture is stirred for 30 minutes, while slowly heating to room temperature. Subsequently, Pd(PPh₃)₄ (134 mg, 0.1159 mmol, 0.20 eq) and Compound 1-3 (204.6 mg, 0.5793 mmol, 1.00 eq) are added thereto. The obtained mixture is heated to 120° C. and stirred overnight. After checking the reaction progress with TLC (EA:Hex=1:20), the reaction is terminated with a NH₄Cl aqueous solution. An organic layer is extracted with CH₂Cl₂ and an aqueous solution of NH₄Cl, and dried with MgSO₄. A product therefrom is filtered with Celite and concentrated under a reduced pressure. 127 mg (65%) of Compound 1-4 is obtained through column chromatography (EA:Hex=1:10 to 1:5).

(4) Synthesizing Compound 1 from Compound 1-4

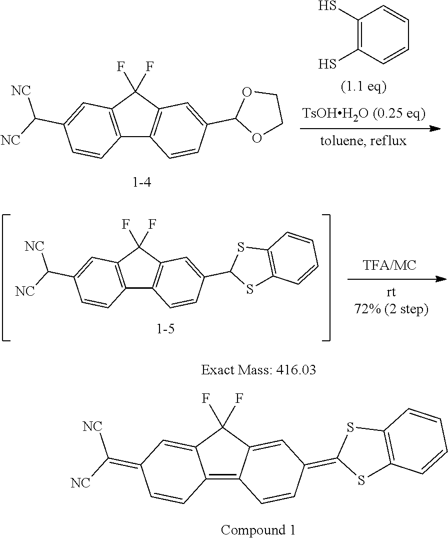

In a 10 ml round-bottomed flask dried with anhydrous flame, Compound 1-4 (61.44 mg, 0.1816 mmol, 1.00 eq) is dissolved in toluene (2 ml). Subsequently, benzenedithiol (28 mg, 0.1997 mmol, 1.10 eq) and TsOH·H₂O (8 mg, 0.0454 mmol, 0.25 eq) are sequentially added to toluene (0.5 ml) at room temperature. The obtained mixture is slowly heated from room temperature to 120° C. and stirred for 4 hours. The reaction solution including Compound 1-5 in the crude state is cooled to room temperature, and 0.5 ml of TFA is added thereto and then, stirred for 1 hour. An organic layer is extracted with CH₂Cl₂ and an aqueous solution of NaHCO₃, dried with MgSO₄, and concentrated under a reduced pressure. A crude material therefrom is washed with ether to obtain 54.45 mg (72%) of Compound 1.

Compound 1 exhibits the following ¹H NMR result and a molecular weight of 415.893 by HRMS (MALDI-TOF):

¹H NMR (400 MHz, TFA-d) δ 8.70-8.67 (m, 2H), 8.58-8.55 (m, 2H), 8.23-8.20 (m, 3H), 8.16-8.11 (m, 2H), 8.23 (d, J=7.6 Hz, 1H); HRMS (MALDI-TOF) found 415.893.

### Synthesis Example 2: Synthesis of Compound 2

Compound 2 is synthesized in the same manner as described in Synthesis Example 1 except that Compound 2-1 is used instead of Compound 1-1 of Synthesis Example 1 as a starting material.

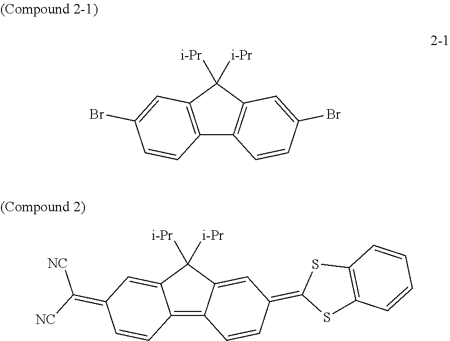 [SMILES: CC(C)C1(C(C)C)c2cc(Br)ccc2-c2ccc(Br)cc21 ; CC(C)C1(C(C)C)c2cc(=C(C#N)C#N)ccc2=c2ccc(=C3Sc4ccccc4S3)cc21]

Herein, Compound 2-1 may be synthesized according to the following reaction scheme by using fluorene as a starting material with reference to the disclosure of Beilstein J. Org. Chem. 2020, 16, 1683 to 1692 and Martin S.; Peter S. Chem. Mater. 2004, 16, 4736 to 4742:

(Reaction Scheme for the Preparation of Compound 2-1)

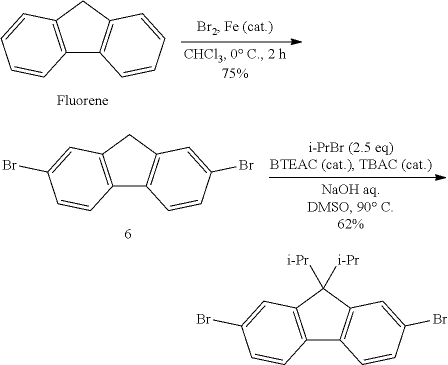

Specifically, in a 250 ml round-bottomed flask dried with anhydrous flame, fluorene (4.0 g, 24.06 mmol, 1.00 eq) and Fe (134 mg, 2.41 mmol, 0.1 eq) are dissolved in 60 ml of CHCl₃ and then, cooled to 0° C., and Br₂ (1.0 M in CHCl₃, 50.52 ml, 50.52 mmol, 2.10 eq) is added thereto in a dropwise fashion and then, stirred for 2 hours. After checking the reaction progress through NMR, the reaction is terminated by using 150 ml of a Na₂S₂O₅ saturated aqueous solution. Subsequently, an organic layer is extracted therefrom by using CH₂Cl₂ and an aqueous solution of Na₂S₂O₅, dried with MgSO₄, and concentrated under a reduced pressure. 8 g of a crude solution obtained therefrom is filter-washed by using EtOH at 0° C. 6.0 g (75%) of 2,7-dibromo-9H-fluorene of a white solid (Compound 6 in the above reaction scheme) as an intermediate is obtained. Compound 6 has the following ¹H NMR result:

¹H NMR (400 MHz, CDCl₃) δ 7.64 (s, 2H), 7.56 (d, J=8.1 Hz, 2H), 7.48 (d, J=8.1 Hz, 2H), 3.82 (s, 2H); ¹³C NMR (100 MHz, CDCl₃) δ 144.75, 139.64, 130.10, 128.29, 128.23, 121.15, 120.90, 36.53.

Subsequently, Compound 6 (2,7-dibromo-9H-fluorene) (390 mg, 1.203 mmol, 1.00 eq) is put in a 25 ml round-bottomed flask and then, dissolved in DMSO (4 ml). Then, 26 mg of bexzyltriethylammonium chloride, 26 mg of tetrabutylammonium chloride, and 2 ml of a 25N NaOH aqueous solution are sequentially added thereto at room temperature, and i-PrBr (0.33 ml, 3.008 mmol, 2.50 eq) is added thereto in a dropwise fashion. The reactant is heated to 100° C. and stirred for 2 hours. Subsequently, after checking the reaction progress through TLC (hexane) and cooling to room temperature, an organic layer is extracted therefrom with CH₂Cl₂ and H₂O, dried with MgSO₄, and concentrated under a reduced pressure. A product therefrom is recrystallized by using EtOH to obtain 310 mg of Compound 2-1 with a yield of 62%. Compound 2-1 has the following ¹H NMR result:
R_{f}: 0.6 (SiO₂, hexane); ¹H NMR (400 MHz, CDCl₃) δ 7.54-7.50 (m, 4H), 7.46 (dd, J=8.1, 1.4 Hz, 2H), 2.55-2.43 (m, 2H), 0.71 (d, J=6.8 Hz, 12H); ¹³C NMR (100 MHz, CDCl₃) δ 151.19, 139.71, 130.05, 127.41, 127.35, 121.11, 120.74, 61.91, 32.96, 17.68.

### Comparative Synthesis Example 1: Synthesis of Compound a

Compound a is synthesized according to Reaction Scheme 2.

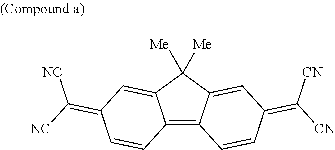 [SMILES: CC1(C)c2cc(=C(C#N)C#N)ccc2=c2ccc(=C(C#N)C#N)cc21]

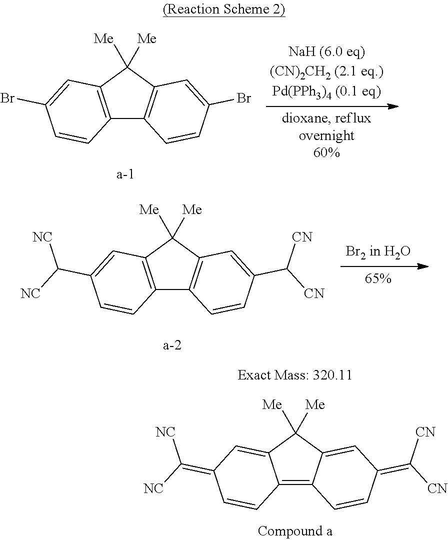 [SMILES: CC1(C)c2cc(Br)ccc2-c2ccc(Br)cc21 ; CC1(C)c2cc(C(C#N)C#N)ccc2-c2ccc(C(C#N)C#N)cc21 ; CC1(C)c2cc(=C(C#N)C#N)ccc2=c2ccc(=C(C#N)C#N)cc21]

Compound a-2 is synthesized with a yield of 60% by using Compound a-1 shown in Reaction Scheme 2 as a starting material through a coupling reaction with 2.1 equivalent of malononitrile under a catalyst amount of Pd(PPh₃)₄ in a dioxane solvent. An oxidation reaction using a Br₂ aqueous solution proceeds, and a solid precipitated therein is extracted, filtered, and washed with ether to synthesize Compound a with a yield of 65%.

Hereinafter, each synthesis process step in Reaction Scheme 2 will be more specifically described.

(1) Synthesis Step of Compound a-2 from Compound a-1

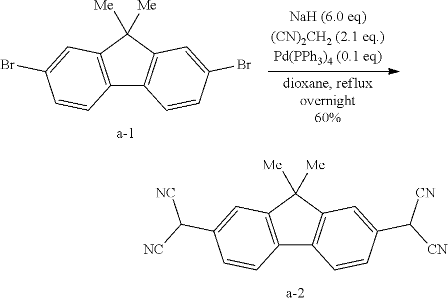 [SMILES: CC1(C)c2cc(Br)ccc2-c2ccc(Br)cc21 ; CC1(C)c2cc(C(C#N)C#N)ccc2-c2ccc(C(C#N)C#N)cc21]

In a 100 ml round-bottomed flask dried with anhydrous flame, (CN)₂CH₂ (0.463 ml, 8.349 mmol, 2.10 eq) is dissolved in dioxane (21 ml) and cooled to 0° C., and 60% NaH (0.954 g, 23.859 mmol, 6.00 eq) is added thereto. The obtained mixture is slowly to room temperature and stirred for 30 minutes. Subsequently, Pd(PPh₃)₄ (0.459 g, 0.397 mmol, 0.10 eq) and Compound a-1 (1.4 g, 3.976 mmol, 1.00 eq) as a starting material are added thereto and then, heated to 120° C. and stirred overnight. After checking the reaction progress with TLC (EA:Hex=1:20, 1:1), the reaction solution is cooled to room temperature. After terminating the reaction with a NH₄Cl aqueous solution, an organic layer is extracted therefrom by using CH₂Cl₂ and an aqueous solution of NH₄Cl, and dried with MgSO₄. The dried product is filtered with Celite, and concentrated under a reduced pressure. Subsequently, the obtained product is treated through column chromatography (MC) and recrystallized with ether/Hex to obtain 650 mg (51%) of Compound a-2.

(2) Synthesis Step of Compound a from Compound a-2

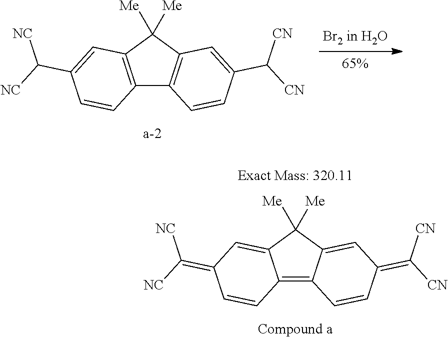 [SMILES: CC1(C)c2cc(C(C#N)C#N)ccc2-c2ccc(C(C#N)C#N)cc21 ; CC1(C)c2cc(=C(C#N)C#N)ccc2=c2ccc(=C(C#N)C#N)cc21]

After putting a Br₂ saturated aqueous solution (5 ml) in a 10 ml round-bottomed flask, Compound a-2 (150 mg, 0.4653 mmol, 1.00 eq) is added thereto at room temperature. After reacting them at room temperature for 3 hours and then, checking the reaction progress with TLC (MC), a solid precipitated therein is filtered and sequentially washed with H₂O and diethyl ether. Subsequently, the product is vacuum-dried to obtain 95 mg (50%) of Compound a. Compound a has the following ¹H NMR result and a molecular weight of about 319.697 by MALDI-TOF:

¹H NMR (400 MHz, TFA-d) δ 7.72 (d, J=9.2 Hz, 2H), 7.57 (d, J=8.9 Hz, 2H), 7.40 (s, 2H), 1.51 (s, 6H); HRMS (MALDI-TOF) found 319.697.

### Synthesis Example 3: Synthesis of Compound 3

Compound 3 is synthesized through a reaction process shown in Reaction Scheme 3.

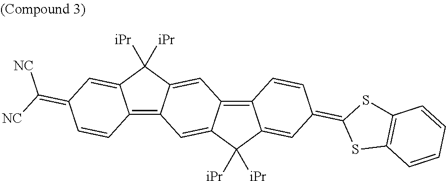 [SMILES: CC(C)C1(C(C)C)c2cc(=C(C#N)C#N)ccc2=c2cc3c(cc21)=c1ccc(=C2Sc4ccccc4S2)cc1C3(C(C)C)C(C)C]

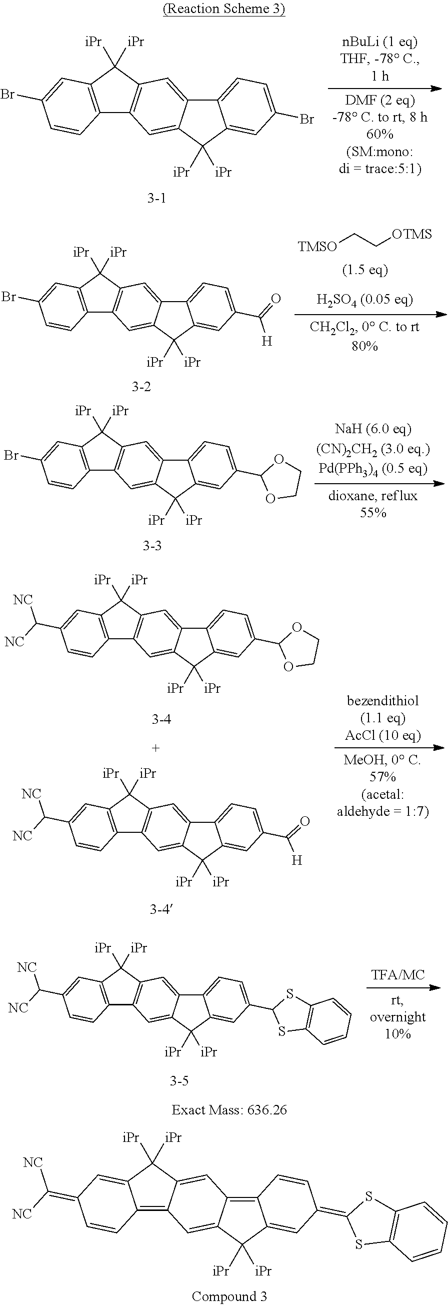

A product 3-2 with a yield of 60% is synthesized by using Compound 3-1 shown in Reaction Scheme 3 as a starting material and controlling an equivalent and a dropping rate of n-BuLi in a THE solvent. Through the above synthesis scheme, Compound 3-3 of acetal is synthesized with a yield of 80%, and a mixed product of Compound 3-4 with an acetal structure and Compound 3-4′ with an deprotected aldehyde structure in a ratio of 1:7 is synthesized with a yield of 55% through a coupling reaction of with malononitrile under a catalyst amount of Pd(PPh₃)₄. It is confirmed that Compound 3-5 is synthesized with a yield of 57% from the mixture of the compound 3-4 and the compound 3-4′ through substitution and protection reaction with benzene-1,2-dithiol. The compound 3-5 is TFA-treated at room temperature to synthesize Compound 3 with a yield of 60%.

Hereinafter, the reaction of each step in Reaction Scheme 3 is described in detail.

(0) Synthesis Step of Compound 3-1 as Starting Material

In Reaction Scheme 3, Compound 3-1 as a starting material is synthesized according to the following reaction scheme with reference to Advanced Synthesis & Catalysis (2010), 352(18), 3267 to 3274, Organic Letters (2005), 7(5), 795 to 797, and Chemistry of Materials (2004), 16(23), 4736 to 4742.

(Synthesis Reaction Scheme of Compound 3-1)

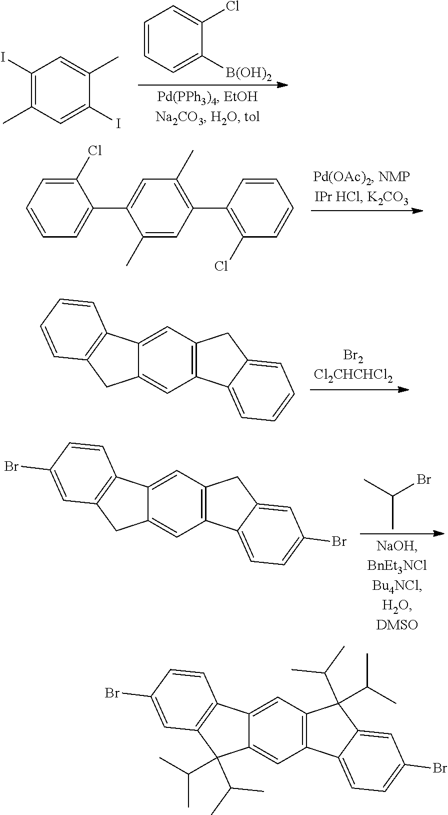

(1) Synthesis step of Compound 3-2 from Compound 3-1

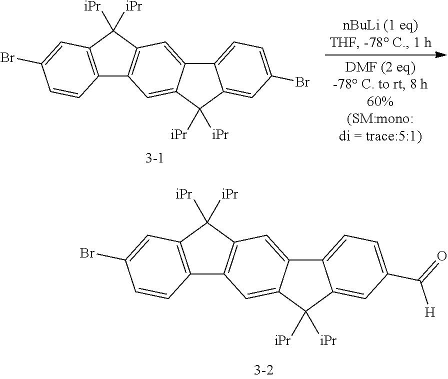 [SMILES: CC(C)C1(C(C)C)c2cc(Br)ccc2-c2cc3c(cc21)-c1ccc(Br)cc1C3(C(C)C)C(C)C ; CC(C)C1(C(C)C)c2cc(Br)ccc2-c2cc3c(cc21)-c1ccc(C=O)cc1C3(C(C)C)C(C)C]

In a 250 ml round-bottomed flask dried with anhydrous flame, Compound 3-1 (3.0 g, 5.168 mmol, 1.00 eq) is dissolved in THE (75 ml). After cooling to −78° C., 1.16 M n-BuLi (4.45 ml, 5.168 mmol, 1.00 eq) is slowly (0.01 ml/min) is added thereto. After stirring at −78° C. for 1 hour, DMF (0.80 ml, 10.336 mmol, 2.00 eq) is added thereto. The obtained mixture is slowly heated to room temperature from −78° C. and stirred overnight. After checking the reaction progress with TLC (EA:Hex=1:20), the reaction is terminated by using a NH₄Cl aqueous solution. Subsequently, an organic layer is extracted therefrom with CH₂Cl₂ and an aqueous solution of NH₄Cl, and dried with Na₂SO₄. Then, 1.62 g of Compound 3-2 of monoaldehyde is obtained with a yield of 60% through column chromatography (Hex to EA:Hex=1:20). R_{f}: 0.3 (SiO₂, hexane/EtOAc=20:1). Compound 3-2 has the following ¹H NMR result:

¹H NMR (400 MHz, CDCl₃) δ 10.05 (s, 1H), 7.93 (s, 1H), 7.87 (q, J=8.1 Hz, 2H), 7.76 (t, J=6.8 Hz, 1H), 7.70 (s, 1H), 7.64-7.59 (m, 1H), 7.57 (s, 1H), 7.49 (d, J=8.1 Hz, 1H), 2.69-2.52 (m, 4H), 0.76 (s, 24H).

(2) Synthesis step of Compound 3-3 from Compound 3-2

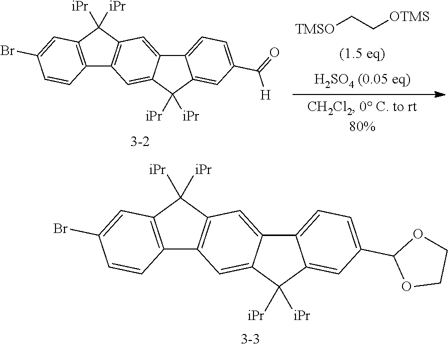

In a 25 ml round-bottomed flask dried with anhydrous flame, Compound 3-2 (750 mg, 1.4177 mmol, 1.00 eq) is dissolved in MC (10 ml), and H₂SO₄ (15 ul, 0.2835 mmol, 0.20 eq) and (TMSOCH₂)₂ (1.79 ml, 5.671 mmol, 4.00 eq) are sequentially added thereto at room temperature. After stirring at room temperature for 10 minutes and then, checking the reaction progress with TLC(EA:Hex=1:20), the reaction is terminated by using triethylamine. An organic layer is extracted therefrom with CH₂Cl₂ and H₂O and then dried with MgSO₄. Subsequently, 650 mg of Compound 3-3 is obtained with a yield of 80% through column chromatography (EA:Hex=1:20). R_{f}: 0.25 (SiO₂, hexane/EtOAc=20:1). Compound 3-3 has the following ¹H NMR result:

¹H NMR (400 MHz, CDCl₃) δ 7.72 (d, J=8.0 Hz, 1H), 7.68 (s, 1H), 7.65 (s, 1H), 7.58 (d, J=7.9 Hz, 1H), 7.54 (s, 1H), 7.51 (s, 1H), 7.49-7.43 (m, 2H), 5.87 (s, 1H), 4.23-4.13 (m, 2H), 4.13-4.01 (m, 2H), 2.65-2.50 (m, 4H), 0.79-0.65 (m, 24H).

(3) Synthesis Step of Compound 3-4 and Compound 3-4′ from Compound 3-3

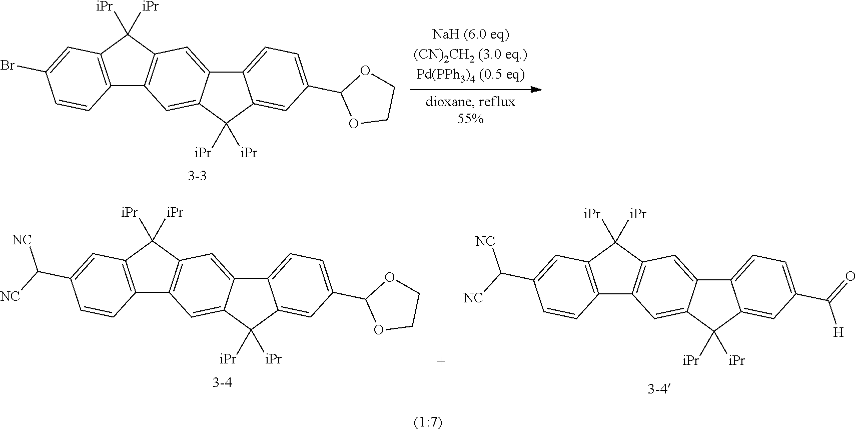 [SMILES: CC(C)C1(C(C)C)c2cc(Br)ccc2-c2cc3c(cc21)-c1ccc(C2OCCO2)cc1C3(C(C)C)C(C)C ; CC(C)C1(C(C)C)c2cc(C(C#N)C#N)ccc2-c2cc3c(cc21)-c1ccc(C2OCCO2)cc1C3(C(C)C)C(C)C ; CC(C)C1(C(C)C)c2cc(C=O)ccc2-c2cc3c(cc21)-c1ccc(C(C#N)C#N)cc1C3(C(C)C)C(C)C]

In a 100 ml round-bottomed flask dried with anhydrous flame, (CN)₂CH₂ (138 mg, 2.0919 mmol, 3.00 eq) is dissolved in 1,4-dioxane (20 ml). After cooling to 0° C., 60% NaH (167 mg, 4.1839 mmol, 6.00 eq) is added thereto and then stirred for 20 minutes, while slowly heating to room temperature. Subsequently, Pd(PPh₃)₄ (403 mg, 0.3486 mmol, 0.50 eq) and Compound 3-3 (400 mg, 0.6973 mmol, 1.00 eq) are added thereto and then, heated to 120° C. and stirred for 5 hours. After checking the reaction progress with TLC (EA:Hex=1:20, 1:5), the reaction is terminated by using H₂O. Subsequently, an organic layer is extracted therefrom with CH₂Cl₂ and a NH₄Cl aqueous solution, dried with MgSO₄, and concentrated under a reduced pressure. 199 mg of Compound 3-4 and Compound 3-4′ as a mixture of an acetal structure and an aldehyde structure in a ratio of 1:7 is obtained with a yield of 55% through column chromatography (MC:Hex=1:1 to 3:1). R_{f}: 0.25 (SiO₂, hexane/EtOAc=5:1)

(4) Synthesis Step of Compound 3-5 from a Mixture of Compound 3-4 and Compound 3-4′

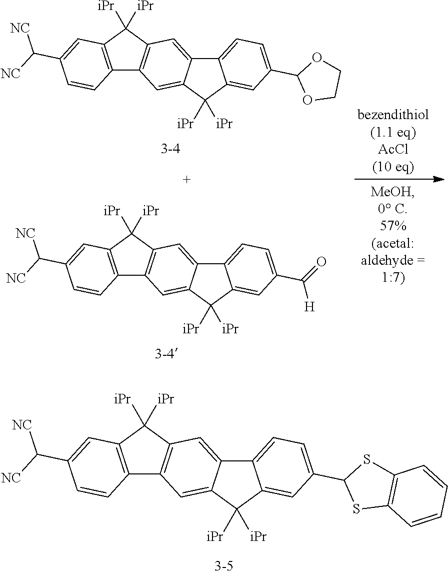 [SMILES: CC(C)C1(C(C)C)c2cc(C(C#N)C#N)ccc2-c2cc3c(cc21)-c1ccc(C2OCCO2)cc1C3(C(C)C)C(C)C ; CC(C)C1(C(C)C)c2cc(C=O)ccc2-c2cc3c(cc21)-c1ccc(C(C#N)C#N)cc1C3(C(C)C)C(C)C ; CC(C)C1(C(C)C)c2cc(C(C#N)C#N)ccc2-c2cc3c(cc21)-c1ccc(C2Sc4ccccc4S2)cc1C3(C(C)C)C(C)C]

MeOH (2.3 ml) is put in a 5 ml round-bottomed flask dried with anhydrous flame and cooled to −20° C. Subsequently, acetyl chloride (0.23 ml, 3.3203 mmol, 10 eq) is added thereto in a dropwise fashion and then stirred for 5 minutes. The mixture (173 mg, 0.3320 mmol 1.00 eq) of Compound 3-4 and Compound 3-4′ in a ratio of 1:7 ratio and benzenedithiol (52 mg, 0.3652 mmol, 1.10 eq) are sequentially added thereto. Subsequently, after slowly heating from −20° C. to 0° C., stirring for 2 hours, and checking the reaction progress with TLC (EA:Hex=1:3), the reaction is terminated with a NaHCO₃ aqueous solution. An organic layer is extracted therefrom by using CH₂Cl₂ and a NaHCO₃ aqueous solution (R_{f}: 0.3 (SiO₂, hexane/EtOAc=5:1), dried with Na₂SO₄, and concentrated under a reduced pressure. 121 mg of Compound 3-5 is obtained with a yield of 57% through recrystallization with ether and Hex. The compound 3-5 has the following ¹H NMR result and a molecular weight of about 639.86 by MALDI-TOF:

¹H NMR (400 MHz, CDCl₃) δ 7.80 (d, J=7.6 Hz, 1H), 7.72-7.64 (m, 3H), 7.56 (d, J=6.9 Hz, 1H), 7.50 (s, 1H), 7.47 (d, J=7.5 Hz, 1H), 7.24-7.18 (m, 2H), 7.10-7.04 (m, 2H), 6.37 (s, 1H), 5.15 (s, 1H), 2.67-2.51 (m, 4H), 0.79-0.66 (m, 24H); HRMS (MALDI-TOF) found 639.86.

(5) Synthesis Step of Compound 3 from Compound 3-5

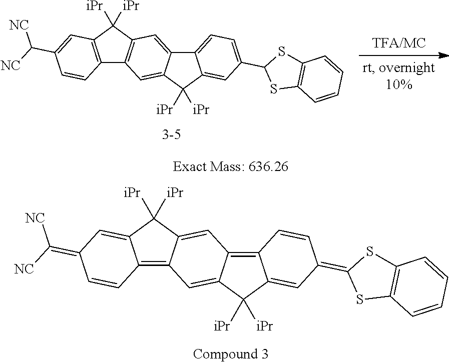 [SMILES: CC(C)C1(C(C)C)c2cc(C(C#N)C#N)ccc2-c2cc3c(cc21)-c1ccc(C2Sc4ccccc4S2)cc1C3(C(C)C)C(C)C ; CC(C)C1(C(C)C)c2cc(=C(C#N)C#N)ccc2=c2cc3c(cc21)=c1ccc(=C2Sc4ccccc4S2)cc1C3(C(C)C)C(C)C]

Compound 3-5 (25 mg, 0.039 mmol) is put in a 5 ml round-bottomed flask at room temperature, and 2 ml of TFA/MC(=1:1) is added thereto and then stirred at room temperature overnight. The solvent is removed from the reaction solution by using a rotary. A solid obtained therefrom is washed with diethyl ether to obtain 2.55 mg of Compound 3 with a yield of 10%. Compound 3 has the following H NMR result and a molecular weight of about 636.076 by MALDI-TOF:

¹H NMR (400 MHz, TFA-d) δ 8.29-8.24 (m, 2H), 8.16 (d, J=6.1 Hz, 2H), 7.97 (d, J=10.6 Hz, 2H), 7.90 (s, 1H), 7.89-7.82 (m, 2H), 7.59 (s, 1H), 7.50 (d, J=7.0 Hz, 1H), 2.80-2.53 (m, 4H), 0.79 (d, J=6.1 Hz, 12H), 0.75-0.66 (m, 12H); HRMS (MALDI-TOF) found 636.076.

### Preparation Synthesis Example: Synthesis of Intermediate Compounds

### Preparation Synthesis Example 1: Synthesis of Compound 4-1

Compound 4-1 is prepared according to Reaction Scheme 4 with reference to the method disclosed in Advanced Synthesis & Catalysis (2010), 352(18), 3267 to 3274.

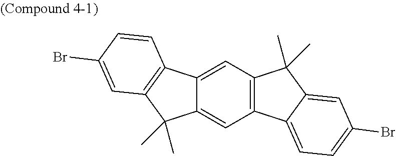 [SMILES: CC1(C)c2cc(Br)ccc2-c2cc3c(cc21)-c1ccc(Br)cc1C3(C)C]

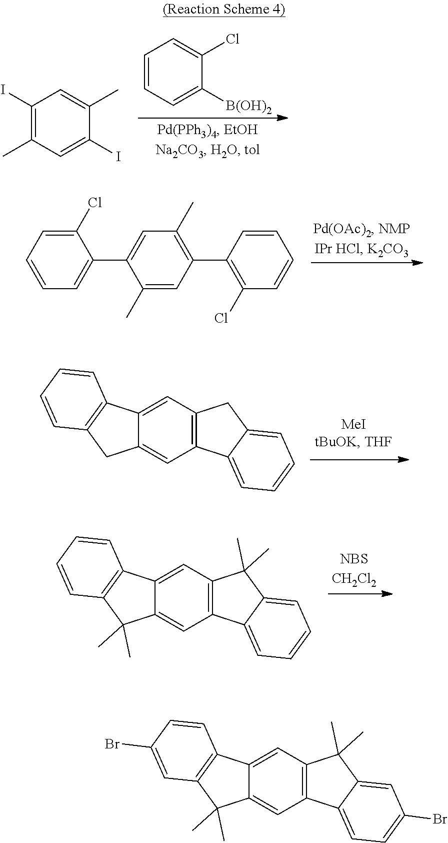

When Compound 4-1 instead of Compound 3-1 is used as a starting material in the same method as described in Synthesis Example 3, prepared is a compound according to some example embodiments, in which a dimethyl group is substituted instead of a diisopropyl group substituted in the rings of Compound 3 of Synthesis Example 3.

### Preparation Synthesis Example 2: Synthesis of Compound 5-1

Compound 5-1 is prepared according to Reaction Scheme 5.

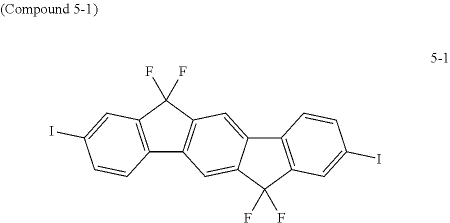 [SMILES: FC1(F)c2cc(I)ccc2-c2cc3c(cc21)-c1ccc(I)cc1C3(F)F]

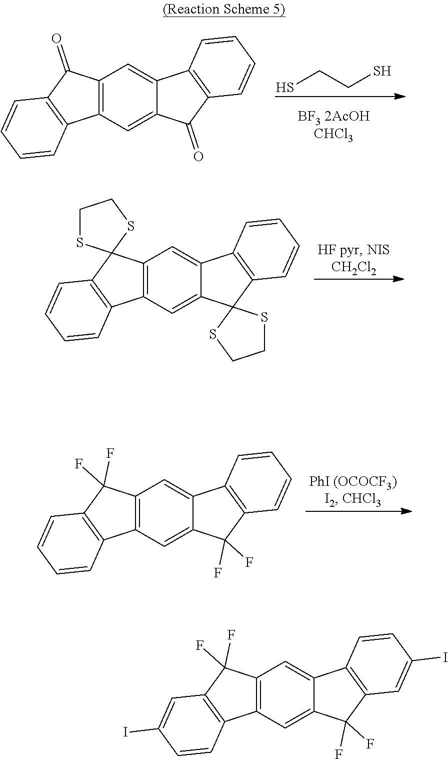

When Compound 5-1 instead of Compound 3-1 is used as a starting material in the same method as described in Synthesis Example 3, prepared a compound according to some example embodiments, in which a difluoro group is substituted instead of a diisopropyl group substituted in the rings of Compound 3 of Synthesis Example 3.

### Preparation Synthesis Example 3: Synthesis of Compound 6-1

Compound 6-1 is prepared according to Reaction Scheme 6.

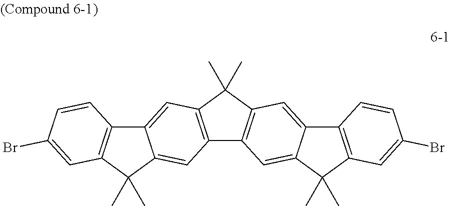 [SMILES: CC1(C)c2cc(Br)ccc2-c2cc3c(cc21)-c1cc2c(cc1C3(C)C)-c1ccc(Br)cc1C2(C)C]

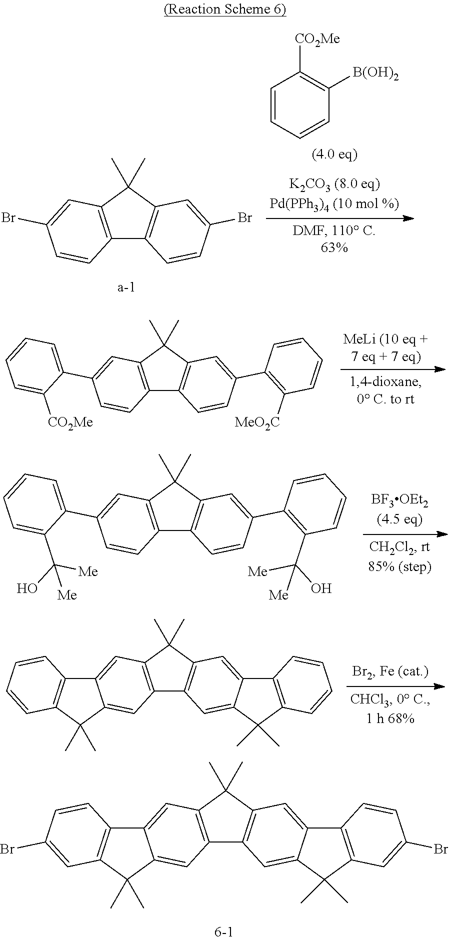

Hereinafter, each reaction step of Reaction Scheme 6 is described in detail.

(1) Synthesis Step of Compound 6-1-1 from Compound a-1

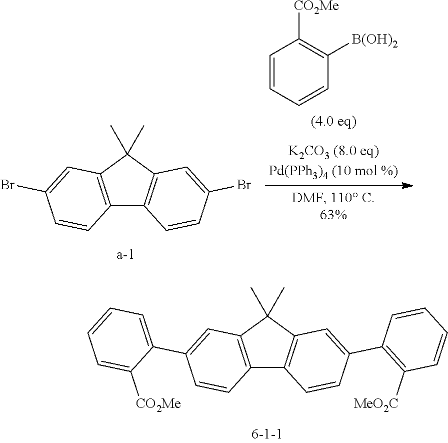 [SMILES: COC(=O)c1ccccc1B(O)O ; CC1(C)c2cc(Br)ccc2-c2ccc(Br)cc21 ; COC(=O)c1ccccc1-c1ccc2c(c1)C(C)(C)c1cc(-c3ccccc3C(=O)OC)ccc1-2]

In a 250 ml round-bottomed flask dried with anhydrous flame, 2,7-dibromo-9,9-dimethyl-9H-fluorene (5.0 g, 14.202 mmol, 1.00 eq), 2-(Methoxycarbonyl)phenylboronic acid (10.224 g, 56.808 mmol, 4.00 eq), K₂CO₃ (15.702 g, 113.617 mmol, 8.00 eq), and Pd(PPh₃)₄ (1.641 g, 1.420 mmol, 10 mol %) are sequentially dissolved in DMF (150 ml). After purging with nitrogen for 10 minutes to remove internal air, the reaction solution is heated to 110° C. and stirred overnight. After checking the reaction progress with TLC (EA:Hex=1:5) and then, cooling the reaction solution to room temperature, the reaction is terminated by using H₂O. Subsequently, DMF is removed by using an air gun. An organic layer is extracted therefrom with CH₂Cl₂ and an aqueous solution of NaCl, dried with MgSO₄, Celite-filtered, and concentrated under a reduced pressure. Subsequently, 4.1 g of a targeted ester compound 6-1-1 is obtained with a yield of 63% through column chromatography (dichloromethane:hexane=3:1). Compound 6-1-1 has the following ¹H NMR result:

R_{f}: 0.5 (SiO₂, hexane/EtOAc=5:1); ¹H NMR (400 MHz, CDCl₃) δ 7.82 (d, J=7.3 Hz, 2H), 7.77 (d, J=7.4 Hz, 2H), 7.56 (t, J=7.3 Hz, 2H), 7.48 (d, J=8.1 Hz, 2H), 7.43 (t, J=7.2 Hz, 2H), 7.37 (s, 2H), 7.33 (d, 2H), 3.62 (s, 6H), 1.52 (s, 6H).

(2) Synthesis Step of Compound 6-1-2 from Compound 6-1-1

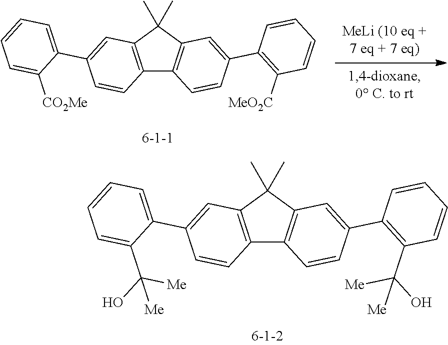 [SMILES: COC(=O)c1ccccc1-c1ccc2c(c1)C(C)(C)c1cc(-c3ccccc3C(=O)OC)ccc1-2 ; CC(C)(O)c1ccccc1-c1ccc2c(c1)C(C)(C)c1cc(-c3ccccc3C(C)(C)O)ccc1-2]

In a 250 ml round-bottomed flask dried with anhydrous flame, Compound 6-1-1 (3.7 g, 7.999 mmol, 1.00 eq) is dissolved in THE (114 ml). After cooling to 0° C., CH₃Li (3.1 M in diethoxymethane) (25.8 ml, 79.99 mmol, 10.00 eq) is added thereto in a dropwise fashion. The mixture is slowly heated to room temperature and stirred overnight. After checking the reaction through TLC (ethyl acetate:hexane=1:5), the reaction is terminated by using an NH₄Cl saturated aqueous solution. An organic layer is extracted therefrom with CH₂Cl₂ and a saturated aqueous solution of NH₄Cl, dried with Na₂SO₄, and concentrated under a reduced pressure. The obtained crude compound is dissolved in THE (114 ml) in the 250 ml round-bottomed flask dried with anhydrous flame. After cooling to 0° C., CH₃Li (3.1 M in diethoxymethane) (18.06 ml, 55.99 mmol, 7.00 eq) is added thereto in a dropwise fashion. The obtained mixture is slowly heated to room temperature and stirred overnight. After examining the reaction for one day, carrying out work-up in the same manner, and once more reacting the obtained crude compound with 7.0 eq of MeLi, the reaction is terminated by using an NH₄Cl saturated aqueous solution. An organic layer is extracted therefrom by using CH₂Cl₂ and an NH₄Cl saturated aqueous solution, dried with Na₂SO₄, and concentrated under a reduced pressure to obtain a crude alcohol compound 6-1-2. Compound 6-1-2 is used in the subsequent reaction without additional purification and has the following ¹H NMR result:

R_{f}: 0.3 (SiO₂, hexane/EtOAc=3:1); ¹H NMR (400 MHz, CDCl₃) δ 7.76 (d, J=7.9 Hz, 2H), 7.69 (d, J=8.0 Hz, 2H), 7.40 (s, 2H), 7.37 (d, J=7.9 Hz, 2H), 7.34-7.27 (m, 6H), 7.16 (d, J=7.4 Hz, 2H), 1.49 (s, 18H).

(3) Synthesis Step of Compound 6-1-3 from Compound 6-1-2

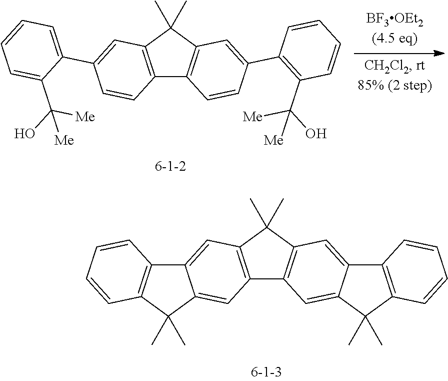

In a 100 ml round-bottomed flask dried with anhydrous flame, the alcohol compound 6-1-2 (5.1 g, 11.024 mmol, 1.00 eq) is dissolved in CH₂Cl₂ (55.1 ml). After cooling to 0° C., BF₃OEt₂ (6.12 ml, 49.609 mmol, 4.50 eq) is added dropwise thereto and then stirred at room temperature for 30 minutes. After checking the reaction through TLC (ethyl acetate:hexane=1:3), the reaction is terminated with TEA. An organic layer is extracted therefrom with CH₂Cl—₂ and H₂O, dried with MgSO₄, and concentrated under a reduced pressure. After adding 15 mL of methanol thereto at 0° C. and then, stirring at room temperature for 1 hour, a solid therefrom is filtered to obtain 4.0 g of Compound 6-1-3 with a yield of 85% in the 2^{nd} step from Compound 6-1-1. Compound 6-1-3 has the following ¹H NMR result:

R_{f}: 0.8 (SiO₂, hexane/EtOAc=3:1); ¹H NMR (400 MHz, CDCl₃) δ 7.82 (s, 2H), 7.78 (d, J=5.8 Hz, 2H), 7.77 (s, 1H), 7.45 (d, J=6.7 Hz, 2H), 7.36 (t, J=7.5 Hz, 2H), 7.31 (t, J=7.5 Hz, 3H), 1.62 (s, 6H), 1.57 (s, 12H), 1.54 (s, 6H).

(4) Synthesis Step of Compound 6-1 from Compound 6-1-3

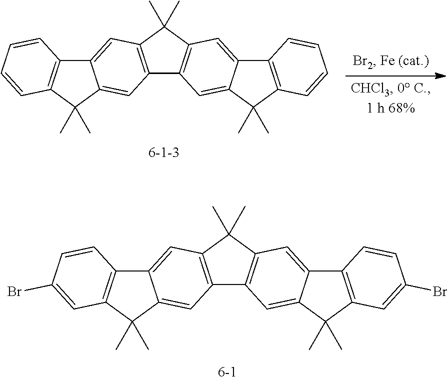

In a 25 ml round-bottomed flask dried with anhydrous flame, Compound 6-1-3 (1.0 g, 2.344 mmol, 1.00 eq) is dissolved in CHCl₃ (7 ml). After cooling to 0° C., Br₂ (a 1.0 M solution in CHCl₃) (4.92 ml, 4.92 mmol, 2.10 eq) is added dropwise thereto and then, stirred for 2 hours. After checking the reaction through TLC (ethyl acetate:hexane=1:3), the reaction is terminated by using aq.Na₂S₂O₅. An organic layer is extracted therefrom with CH₂Cl—₂ and H₂O, dried with MgSO₄, and concentrated under a reduced pressure. After washing with ether:Hex (1:12) at 0° C. and stirring at room temperature for 1 hour, a solid is filtered therefrom to obtain 938 mg of Compound 6-1 with a yield of 68%. Compound 6-1 has the following ¹H NMR result:
R_{f}: 0.8 (SiO₂, hexane/EtOAc=3:1); ¹H NMR (400 MHz, CDCl₃) δ 7.79 (s, 2H), 7.73 (s, 2H), 7.63 (d, J=7.6 Hz, 2H), 7.56 (s, 2H), 7.48 (d, J=8.9 Hz, 2H), 1.60 (s, 6H), 1.56 (s, 12H).

### Preparation Synthesis Example 4: Synthesis of Compound 7-1

Compound 7-1 is prepared according to Reaction Scheme 7.

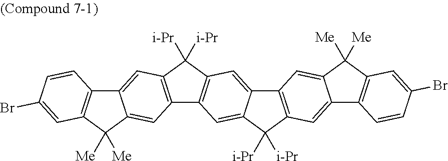 [SMILES: CC(C)C1(C(C)C)c2cc3c(cc2-c2cc4c(cc21)-c1cc2c(cc1C4(C(C)C)C(C)C)-c1ccc(Br)cc1C2(C)C)C(C)(C)c1cc(Br)ccc1-3]

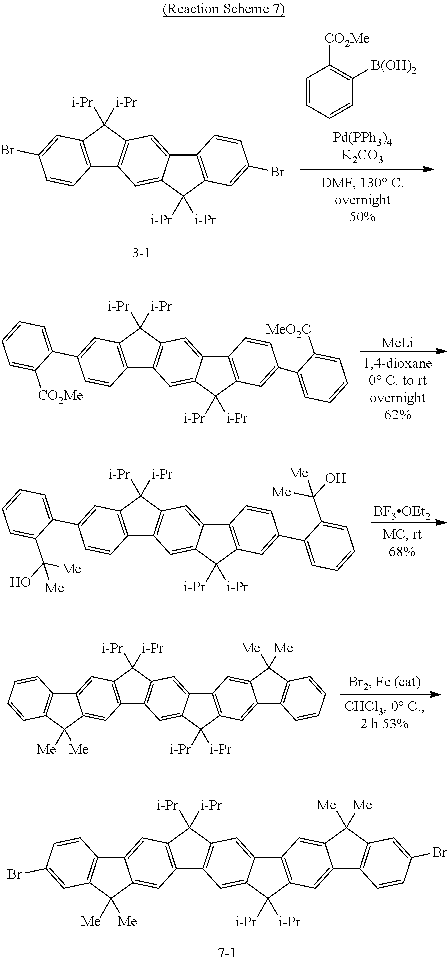

Hereinafter, each reaction step of Reaction Scheme 7 is described in detail.

(1) Synthesis Step of Compound 7-1-1 from Compound 3-1

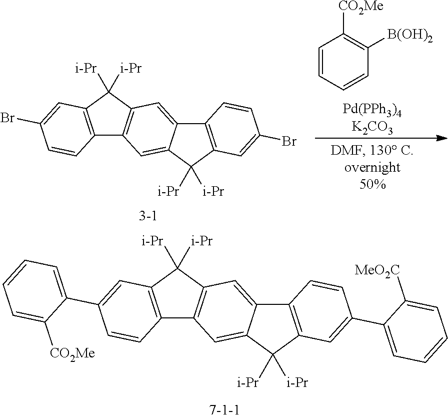 [SMILES: CC(C)C1(C(C)C)c2cc(Br)ccc2-c2cc3c(cc21)-c1ccc(Br)cc1C3(C(C)C)C(C)C ; COC(=O)c1ccccc1B(O)O ; COC(=O)c1ccccc1-c1ccc2c(c1)C(C(C)C)(C(C)C)c1cc3c(cc1-2)C(C(C)C)(C(C)C)c1cc(-c2ccccc2C(=O)OC)ccc1-3]

In a two-neck round-bottomed flask dried with anhydrous flame, Compound 3-1 (5 g, 8.61 mmol, 1.0 eq) is dissolved in DMF (100 mL, 0.086 M). Subsequently, boronic acid (4.65 g, 25.84 mmol, 3.0 eq), Pd(Ph₃)₄ (1 g, 0.86 mmol, 0.1 eq), and K₂CO₃ (7.1 g, 51.68 mmol, 6.0 eq) are sequentially added thereto and then refluxed at 130° C. After examining the reaction for one day, when the reaction is completed, after slowly cooling to room temperature, DMF is all removed therefrom by blowing air. After adding 150 mL of MC to a crude compound therefrom and then, stirring at room temperature for 3 hours, a solid (product) not dissolved therein is filtered therefrom. After removing the solvent of a filtrate therefrom, Compound 7-1-1 with a yield of 50% is obtained by once more conducting recrystallization with MC/Hex/ether. Compound 7-1-1 has the following ¹H NMR result:

¹H NMR (400 MHz, CDCl₃) δ 7.82 (d, J=7.6 Hz, 2H), 7.76 (d, J=7.7 Hz, 2H), 7.73 (s, 2H), 7.57-7.535 (t, J=7.6, 2H), 7.47-7.403 (dt, J=7.6, 4H), 7.35 (s, 3H), 7.32 (d, J=1.5 Hz, 3H), 3.64 (s, 6H), 0.79 (dd, J=14.4, 6.5 Hz, 24H).

(2) Synthesis Step of Compound 7-1-2 from Compound 7-1-1

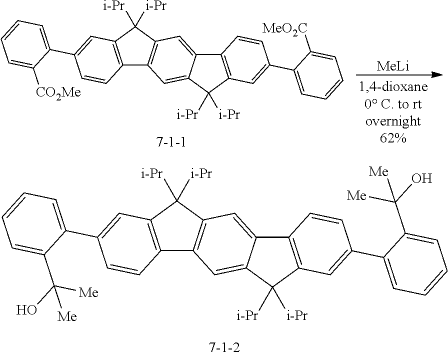 [SMILES: "MeLi" ; COC(=O)c1ccccc1-c1ccc2c(c1)C(C(C)C)(C(C)C)c1cc3c(cc1-2)C(C(C)C)(C(C)C)c1cc(-c2ccccc2C(=O)OC)ccc1-3 ; CC(C)C1(C(C)C)c2cc(-c3ccccc3C(C)(C)O)ccc2-c2cc3c(cc21)-c1ccc(-c2ccccc2C(C)(C)O)cc1C3(C(C)C)C(C)C]

In a round-bottomed flask dried with anhydrous flame, Compound 7-1-1 (2.68 g, 3.88 mmol, 1.0 eq) is diluted in 1,4-dioxane (35 mL) and then, cooled to −78° C. Subsequently, a 3.1 M MeLi solution in diethoxymethane (12.5 mL, 38.80 mmol, 10.0 eq) is slowly added thereto in a dropwise fashion and then, slowly heated to room temperature. After examining the reaction and then terminating the reaction with aq. NH₄Cl reaction, the resultant is extracted with MC and water and then, dried with MgSO₄. The obtained crude compound is diluted in 48 mL of 1,4-dioxane in the round-bottomed flask dried with anhydrous flame and then cooled to −78° C. A 3.1 M MeLi solution in diethoxymethane (8.8 mL, 27.16 mmol, 7.0 eq) is slowly added thereto in a dropwise fashion and then, slowly heated to room temperature. Upon observing the reaction for one day, the reaction is worked-up in the same manner, and the obtained crude compound is once more reacted with 7.0 equivalent of MeLi to converge it to a product as much as possible. After terminating the reaction with an aqueous solution of NH₄Cl, an organic layer is extracted with MC and water, and a crude compound is obtained therefrom by removing the solvent and washed with MeOH to obtain Compound 7-1-2 with a yield of 62%. Compound 7-1-2 has the following ¹H NMR result:

¹H NMR (400 MHz, CDCl₃) δ 7.75 (s, 3H), 7.71-7.697 (d, 2H), 7.40 (s, 4H), 7.31 (d, J=7.5 Hz, 3H), 7.15 (d, J=6.7 Hz, 2H), 2.61 (s, 4H), 1.54 (d, J=18.0 Hz, 12H), 0.83 (m, 24H).

(3) Synthesis Step of Compound 7-1-3 from Compound 7-1-2

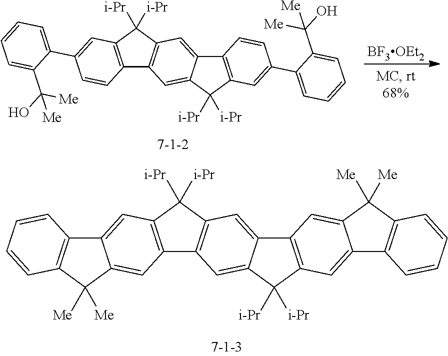

In a round-bottomed flask dried with anhydrous flame, Compound 7-1-2 (1.28 g, 1.86 mmol, 1.0 eq) is dissolved in 37 mL of MC, and BF₃OEt₂ (1.17 mL, 9.29 mmol, 5.0 eq) is slowly added thereto in a dropwise fashion. After checking the reaction with TLC (EA:Hex 1:10) and then, cooling to 0° C., the reaction is terminated with TEA. The resultant is extracted with MC and water and then dried with MgSO₄. After removing the solvent with a rotary reduced pressure evaporator, 15 mL of methanol is added to the obtained crude compound and then, stirred at room temperature for 1 hour. Subsequently, after leaving it in a freezer for a day, a solid produced therein is filtered to obtain Compound 7-1-3 with a yield of 68%. Compound 7-1-3 has the following ¹H NMR result:

¹H NMR (400 MHz, CDCl₃) δ 7.75 (s, 8H), 7.46-7.448 (d, J=7.1 Hz, 2H), 7.39-7.343 (m, 2H), 7.32-7.303 (d, J=6.9 Hz, 2H), 2.75-2.62 (m, 4H), 1.59 (s, 12H), 0.84 (m, 24H).

(4) Synthesis Step of Compound 7-1 from Compound 7-1-3

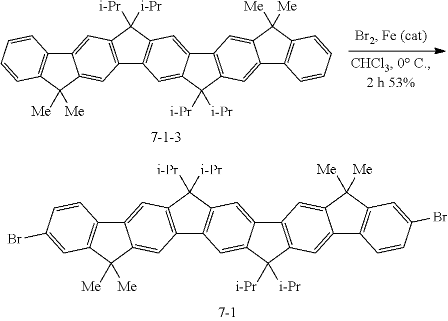

In a round-bottomed flask dried with anhydrous flame, Compound 7-1-3 (800 mg, 1.22 mmol, 1.0 eq) is dissolved in 24 mL of CHCl₃ and then, cooled to 0° C. A 1.0 M Br₂ solution in CHCl₃ (2.56 mL, 2.56 mmol, 2.1 eq) is slowly added thereto in a dropwise fashion. After checking the reaction with TLC (EA:Hex 1:10), the reaction is terminated with aq.Na₂S₂O₅. The resultant is extracted with MC and water and then dried with MgSO₄. The obtained crude compound is washed with ether:Hex 1:9 to obtain Compound 7-1 with a yield of 53%. Compound 7-1 has the following ¹H NMR result:

¹H NMR (400 MHz, CDCl₃) δ 7.77-7.68 (m, 5H), 7.63-7.61 (d, J=8.9 Hz, 2H), 7.56 (s, 2H), 7.48-7.46 (d, J=8.6 Hz, 2H), 2.68 (m, 4H), 1.57-1.55 (d, J=6.5 Hz, 12H), 0.83 (s, 24H).

### Preparation Synthesis Example 5: Synthesis of Compound 8-1

Compound 8-1 is prepared according to Reaction Scheme 8.

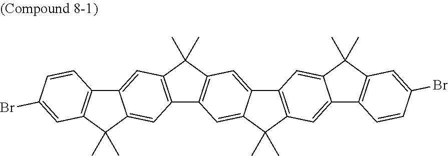 [SMILES: CC1(C)c2cc(Br)ccc2-c2cc3c(cc21)-c1cc2c(cc1C3(C)C)-c1cc3c(cc1C2(C)C)-c1ccc(Br)cc1C3(C)C]

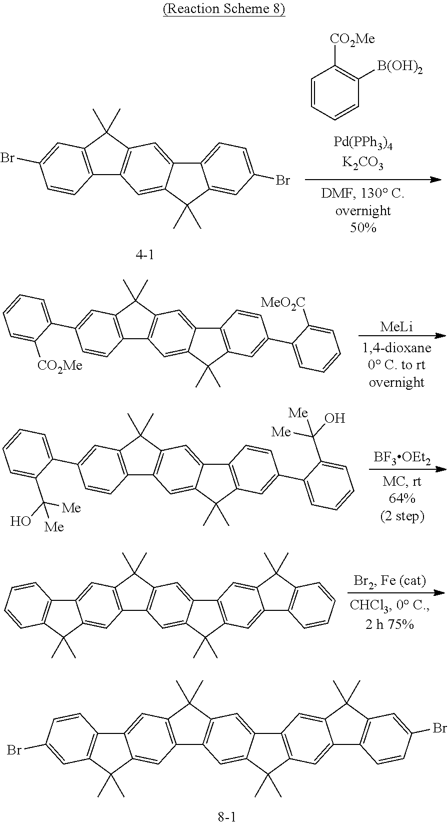

Intermediate 8-1 is synthesized in the same manner as in Reaction Scheme 7 except that Compound 4-1 is used instead of Compound 3-1 as a starting material. Hereinafter, each reaction step of Reaction Scheme 8 is described in detail.

(1) Synthesis Step of Compound 8-1-1 from Compound 4-1

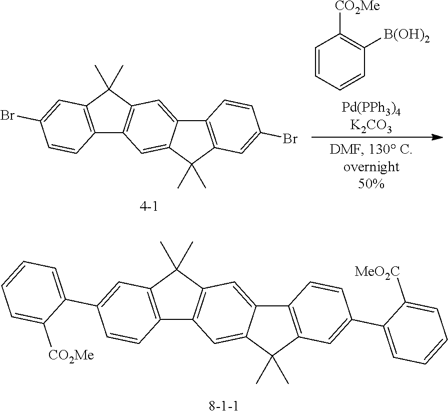 [SMILES: CC1(C)c2cc(Br)ccc2-c2cc3c(cc21)-c1ccc(Br)cc1C3(C)C ; COC(=O)c1ccccc1B(O)O ; COC(=O)c1ccccc1-c1ccc2c(c1)C(C)(C)c1cc3c(cc1-2)C(C)(C)c1cc(-c2ccccc2C(=O)OC)ccc1-3]

In a 250 ml round-bottomed flask dried with anhydrous flame, 2,8-dibromo-6,6,12,12-tetramethyl-6,12-dihydroindeno[1,2-b]fluorene (5.0 g, 10.68 mmol, 1.00 eq) which is Compound 4-1, (methoxycarbonyl)phenylboronic acid (6.73 g, 37.38 mmol, 3.50 eq), K₂CO₃ (10.33 g, 74.76 mmol, 7.00 eq), and Pd(PPh₃)₄ (1.234 g, 1.068 mmol, 10 mol %) are sequentially dissolved in DMF (150 ml). After purging with nitrogen for 10 minutes to remove internal air, the reaction solution is heated to 110° C. and stirred overnight. After checking the reaction with TLC (EA:Hex=1:5), after cooling the reaction solution to room temperature, the reaction is terminated by using H₂O. Subsequently, after removing DMF with an air gun, an organic layer is extracted therefrom with CH₂Cl₂ and a NaCl aqueous solution and then concentrated under a reduced pressure. After adding MC to the obtained crude product and then, stirring at room temperature for 3 hours, a solid (product) not dissolved but left there is filtered. After removing the solvent of a filtrate therefrom, 2.7 g of an ester compound 8-1-1 is obtained with a yield of 50% by once more conducing recrystallization with MC. Compound 8-1-1 has the following ¹H NMR result:

R_{f}: 0.4 (SiO₂, hexane/EtOAc=5:1); ¹H NMR (400 MHz, CDCl₃) δ 7.82 (d, J=4.0 Hz, 2H), 7.81 (d, J=2.5 Hz, 2H), 7.79 (s, 2H), 7.56 (t, J=7.1 Hz, 2H), 7.50 (d, J=7.6 Hz, 2H), 7.43 (t, J=7.6 Hz, 2H), 7.37 (s, 2H), 7.36 (d, J=9.1 Hz, 2H), 3.61 (s, 6H), 1.54 (s, 18H).

(2) Synthesis Step of Compound 8-1-2 from Compound 8-1-1

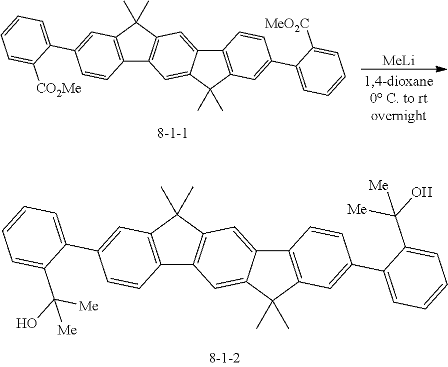 [SMILES: "MeLi" ; COC(=O)c1ccccc1-c1ccc2c(c1)C(C)(C)c1cc3c(cc1-2)C(C)(C)c1cc(-c2ccccc2C(=O)OC)ccc1-3 ; CC(C)(O)c1ccccc1-c1ccc2c(c1)C(C)(C)c1cc3c(cc1-2)C(C)(C)c1cc(-c2ccccc2C(C)(C)O)ccc1-3]

In a 50 ml round-bottomed flask dried with anhydrous flame, Compound 8-1-1 (800 mg, 1.539 mmol, 1.00 eq) is dissolved in 1,4-dioxane (21.98 ml). After cooling to 0° C., CH₃Li (3.1 M in diethoxymethane) (4.97 ml, 15.39 mmol, 10.00 eq) is added thereto in a dropwise fashion. The resultant is slowly heated to room temperature and stirred overnight. After checking the reaction with TLC (ethyl acetate:Hexane=1:3), the reaction is terminated by using a NH₄Cl saturated aqueous solution. An organic layer is extracted therefrom with CH₂Cl₂ and a NH₄Cl saturated aqueous solution and then concentrated under a reduced pressure. The obtained crude compound is put in the 50 ml round-bottomed flask dried with anhydrous flame and dissolved in 1,4-dioxane (21.98 ml). After cooling to 0° C., CH₃Li (3.1M in diethoxymethane) (348 ml, 10.78 mmol, 7.00 eq) is added thereto in a dropwise fashion and then, slowly heated up to room temperature and stirred overnight. After examining the reaction for a day and carrying out work-up in the same manner, the obtained crude compound is once more reacted with 7.0 eq of MeLi, and then, the reaction is terminated by using a NH₄Cl saturated aqueous solution. An organic layer is extracted therefrom with CH₂Cl₂ and an NH₄Cl saturated aqueous solution, dried with Na₂SO₄, and concentrated under a reduced pressure to obtain a crude compound 8-1-2 in the alcohol step. Compound 8-1-2 is used in the subsequent reaction with purification and has the following ¹H NMR result:

R_{f}: 0.3 (SiO₂, hexane/EtOAc=3:1); ¹H NMR (400 MHz, CDCl₃) δ 7.80 (s, 2H), 7.79 (d, J=7.9 Hz, 2H), 7.69 (d, J=7.8 Hz, 2H), 7.40 (s, 2H), 7.37 (d, J=6.3 Hz, 2H), 7.34-7.27 (m, 4H), 7.18 (d, J=7.0 Hz, 2H), 1.54 (s, 12H), 1.50 (s, 12H).

(3) Synthesis Step of Compound 8-1-3 from Compound 8-1-2

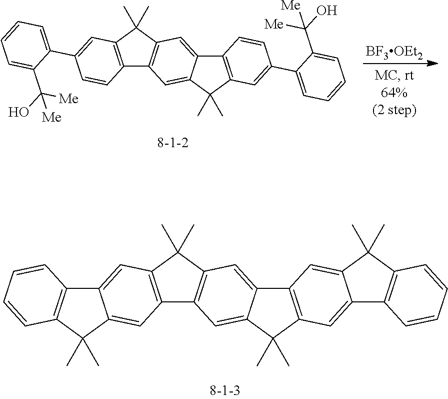

In a 100 ml round-bottomed flask dried with anhydrous flame, Compound 8-1-2 (2.45 g, 4.233 mmol, 1.00 eq) is dissolved in CH₂Cl₂ (42.33 ml). After cooling to 0° C., BF₃OEt₂ (2.61 ml, 21.165 mmol, 5.00 eq) is added dropwise thereto and then, stirred at room temperature for 30 minutes. After checking the reaction with TLC (ethyl acetate:Hexane=1:3), the reaction is terminated with TEA. An organic layer is extracted therefrom with CH₂Cl₂ and H₂O, and concentrated under a reduced pressure. After adding ethyl acetate:hexane (1:12) thereto at 0° C., the mixture is stirred at room temperature for 1 hour, and a solid therein is filtered to obtain 1.46 g of Compound 8-1-3 with a yield of 64% form Compound 8-1-1 in the 2^{nd} step. Compound 8-1-3 has the following ¹H NMR result:

R_{f}: 0.8 (SiO₂, hexane/EtOAc=3:1); ¹H NMR (400 MHz, CDCl₃) δ 7.83 (s, 2H), 7.83 (s, 2H), 7.78 (d, J=5.3 Hz, 2H), 7.77 (s, 2H), 7.45 (d, J=8.1 Hz, 2H), 7.36 (t, J=7.1 Hz, 2H), 7.31 (t, J=6.8 Hz, 2H), 1.64 (s, 12H), 1.54 (s, 12H).

(4) Synthesis Step of Compound 8-1 from Compound 8-1-3

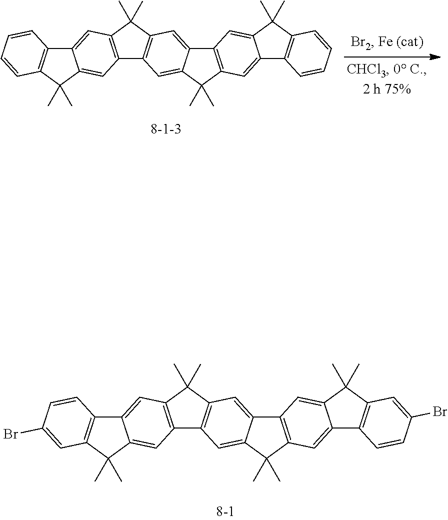

In a 50 ml round-bottomed flask dried with anhydrous flame, Compound 8-1-3 (504 mg, 0.929 mmol, 1.00 eq) is dissolved in CHCl₃ (18.58 ml). After cooling to 0° C., Br₂ (1.0 M solution in CHCl₃) (1.95 ml, 1.95 mmol, 2.10 eq) is added thereto in a dropwise fashion, and stirred for 2 hours. After checking the reaction with TLC (ethyl acetate:hexane=1:3), the reaction is terminated with an aqueous solution of Na₂S₂O₅. An organic layer is extracted therefrom with CH₂Cl₂ and H₂O, and concentrated under a reduced pressure. After adding EtOH thereto at 0° C., the mixture is stirred for 1 hour, and a solid therein is filtered to obtain 488 mg of Compound 8-1 with a yield of 75%. The compound 8-1 has the following ¹H NMR result:
R_{f}: 0.8 (SiO₂, hexane/EtOAc=3:1); ¹H NMR (400 MHz, CDCl₃) δ 7.82 (s, 2H), 7.78 (d, J=32.1 Hz, 2H), 7.74 (s, 2H), 7.63 (s, 2H), 7.60 (d, J=22.2 Hz, 2H), 7.48 (s, 2H), 1.57 (s, 24H).

### Evaluation I: Light Absorption Characteristics

(1) Comparison of Absorption Spectra in Solution of Compounds According to Synthesis Examples and Comparative Synthesis Examples

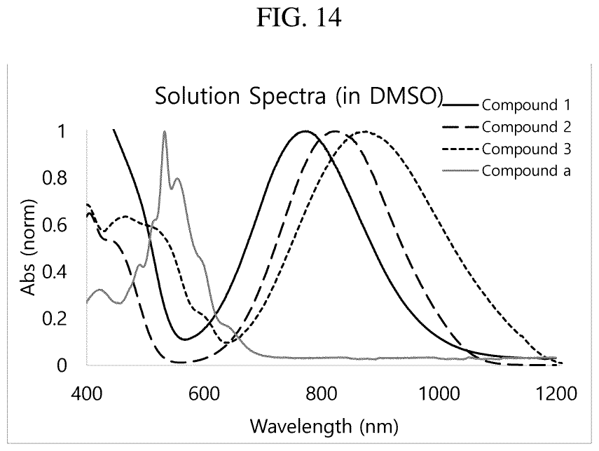

After dissolving Compound 1, Compound 2, Compound 3, and Compound a prepared in Synthesis Example 1, Synthesis Example 2, Synthesis Example 3, and Comparative Synthesis Example 1, respectively, in DMSO (dimethyl sulfoxide), absorption spectra are measured, and the results are shown in FIG. 14.

As seen from FIG. 14, while the maximum peak absorption wavelength of Compound a according to Comparative Synthesis Example 1 is less than 600 nm in the infrared region, the maximum peak absorption wavelengths of Compound 1 and Compound 2 according to some example embodiments in the infrared region exist around 800 nm. In particular, the maximum absorption peak of Compound 1 is less than 800 nm, the maximum absorption peak of Compound 2 is greater than or equal to 800 nm, and the maximum absorption peak of Compound 3 exists around 900 nm. These results show that the compound according to some example embodiments (e.g., Synthesis Examples 1 to 3) has excellent light absorption characteristics in the near-infrared region.

(2) Prediction of Light Absorption Characteristics According to Simulation Calculation Results

The results of calculating the light absorption characteristics (e.g., peak absorption wavelength) of compounds according to some example embodiments are shown in Table 1.

TABLE 1

| | 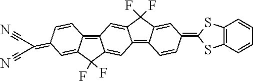 [SMILES: N#CC(C#N)=c1ccc2c(c1)C(F)(F)c1cc3c(cc1=2)C(F)(F)c1cc(=C2Sc4ccccc4S2)ccc1=3] | 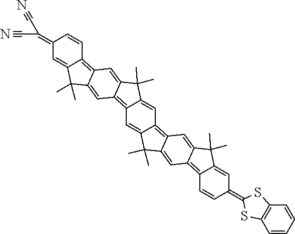 [SMILES: CC1(C)c2cc(=C(C#N)C#N)ccc2=c2cc3c(cc21)=c1cc2c(cc1C3(C)C)=c1cc3c(cc1C2(C)C)=c1ccc(=C2Sc4ccccc4S2)cc1C3(C)C] |
|---|---|---|
| Calculation Method | (nm) | (nm) |
| Exact Mass | 540 | 756 |
| b3lyp/6-311g (d,p) | 800 | 1354 |
| wb97xd/6-311g (d,p) | 842 | 1737 |
| m062x/6-311g (d,p) | 807 | 1471 |
| m11/6-311g (d,p) | 854 | 1895 |
| lc-wpbe/6-311g | 837 | 1884 |
| (d,p) | | |

Referring to Table 1, the compounds according to some example embodiments are low molecular weight substances of less than or equal to 800 g/mol or less and can absorb light in a long wavelength range of greater than or equal to 800 nm. In particular, by increasing the conjugate length, light in a longer wavelength range of greater than or equal to 1,300 nm can be absorbed, and even if the number of repeating units in the conjugate structure increases to 4, the molecular weight is only 756 g/mol, which shows that it can be advantageously applied to the deposition process by maintaining a low molecular weight.

### Examples and Comparative Examples: Manufacture of Photoelectric Devices

ITO is deposited on a glass substrate by sputtering to form an anode with a thickness of 150 nm. Subsequently, the compounds according to Synthesis Examples 1 to 3 and the compound according to Comparative Synthesis Example 1 are co-deposited on the anode at a 1:1 volume ratio with C60, respectively, to form an active layer (photoelectric conversion layer) with a thickness of 150 nm. C60 is deposited on the active layer to form an auxiliary layer with a thickness of 30 nm. ITO is sputtered on the auxiliary layer to form a 7 nm thick cathode, aluminum oxide (Al₂O₃) is deposited on the cathode to form a 50 nm-thick anti-reflection layer, and then sealed with a glass plate to produce photoelectric devices according to Synthesis Examples 1 to 3 and Comparative Synthesis Example 1.

While the inventive concepts have been described in connection with what is presently considered to be practical example embodiments, it is to be understood that the inventive concepts are not limited to such example embodiments. On the contrary, the inventive concepts are intended to cover various modifications and equivalent arrangements included within the spirit and scope of the appended claims.

<Description of symbols>

| | |
|---|---|
| 10, 101: first electrode | 20, 102: second electrode |
| 30, 103: active layer | 40: first auxiliary layer, |
| 45: second auxiliary layer | 50a, 50b, 50c: photo-sensing device |
| 55: charge storage | 70a, 70b, 70c: (blue, red, and green) |
| | color filter |
| 60: lower insulation layer | 80: (upper) insulation layer |
| 85: trench | 95: dual bandpass filter |
| 100, 200: photoelectric device | 100A: first infrared light sensor |
| 110: semiconductor substrate | 120: second infrared light sensor |
| 300, 400, 500, 600, 700, 800, | |
| 900, 950, 970: organic sensor | |

What is claimed is:

1. A compound represented by Chemical Formula 1: 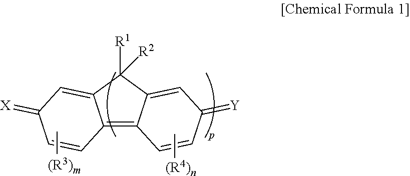
wherein, in Chemical Formula 1,
R¹ and R² are each independently hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³ and R⁴ are each independently deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
m and n are each independently one of integers of 0 to 3,
p is one of integers of 1 to 5, and
X and Y are each independently an electron-acceptor moiety, an electron-donor moiety, or any combination thereof,
provided that, when p is 1 and X and Y are a same electron-acceptor moiety, both R¹ and R² are not simultaneously a substituted or unsubstituted C1 to C30 saturated aliphatic hydrocarbon group,
provided that, when p is 1 and one of X or Y is an electron-acceptor moiety and another one of X or Y is an electron-donor moiety, both R¹ and R² are not simultaneously hydrogen or a methyl group, and
provided that, when p is 2, X and Y do not include a same electron-acceptor moiety.

2. The compound of claim 1, wherein the electron-acceptor moiety is represented by (CN)₂—C═*, wherein * is a portion linked to a position of X in Chemical Formula 1 or a position of Y in Chemical Formula 1.

3. The compound of claim 1, wherein the electron-donor moiety is represented by Chemical Formula 2: 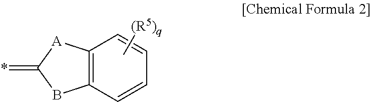
wherein, in Chemical Formula 2,
A and B are each independently oxygen (O), sulfur (S), selenium (Se), or tellurium (Te),
R⁵ is deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to a position of X in Chemical Formula 1 or a position of Y in Chemical Formula 1.

4. The compound of claim 1, wherein
R¹ to R⁴ are each independently deuterium, a halogen, a cyano group, a nitro group, a substituted or unsubstituted C1 to C10 alkyl group, a substituted or unsubstituted C6 to C20 aryl group, or any combination thereof,
m and n are each independently an integer of 0 or 1, and
p is one of integers of 1 to 4.

5. The compound of claim 1, wherein
p is 1,
one of X or Y is an electron-acceptor moiety and another one of X or Y is an electron-donor moiety, and
R¹ and R² are each independently a halogen, a cyano group, a nitro group, a substituted or unsubstituted C2 to C20 alkyl group, a substituted or unsubstituted C6 to C20 aryl group, or any combination thereof.

6. The compound of claim 1, wherein
p is 1,
X and Y are each a same electron-donor moiety,
R¹ and R² are each independently hydrogen, a halogen, a cyano group, a nitro group, a substituted or unsubstituted C1 to C20 alkyl group, a substituted or unsubstituted C6 to C20 aryl group, or any combination thereof, and
R¹ and R² are not simultaneously a substituted or unsubstituted C1 to C20 alkyl group.

7. The compound of claim 1, wherein
p is 2,
X is represented by (CN)₂—C═*, wherein * is a portion linked to a position of X in Chemical Formula 1, and
Y is represented by Chemical Formula 2: 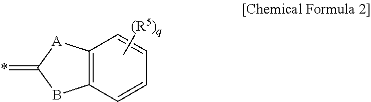
wherein, in Chemical Formula 2,
A and B are each independently oxygen (O), sulfur (S), selenium (Se), or tellurium (Te),
R⁵ is deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to a position of Y in Chemical Formula 1.

8. The compound of claim 1, wherein
p is 2, and
both X and Y are represented by Chemical Formula 2: 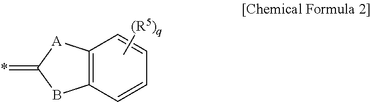
wherein, in Chemical Formula 2,
A and B are each independently oxygen (O), sulfur (S), selenium (Se), or tellurium (Te),
R⁵ is deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to a position of X in Chemical Formula 1 or a position of Y in Chemical Formula 1.

9. The compound of claim 1, wherein
R¹ and R² are each independently a halogen, a substituted or unsubstituted C1 to C5 alkyl group, or any combination thereof,
m and n are each independently 0 or 1,
p is one of integers of 1 to 4,
X and Y are each independently (CN)₂—C═*, 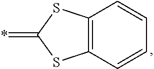 [SMILES: *=C1Sc2ccccc2S1]
or any combination thereof, wherein * is a portion linked to a position of X in Chemical Formula 1 or a position of Y in Chemical Formula 1,
when p is 1, at least one of R¹ or R² is a halogen, and
when p is 2, X and Y are different from each other.

10. The compound of claim 1, wherein Chemical Formula 1 is represented by one or more of Chemical Formulas 1-1 to 1-3: 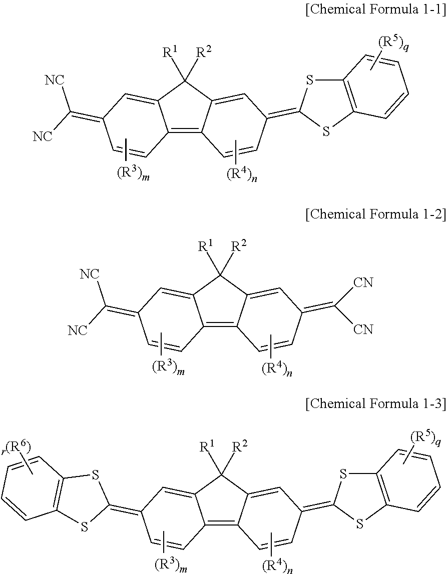
wherein, in Chemical Formulas 1-1 to 1-3,
R¹ and R² are each independently hydrogen, deuterium, a halogen, or a substituted or unsubstituted C1 to C30 alkyl group,
R³ and R⁴ are each independently deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
m and n are each independently one of integers of 0 to 3,
R⁵ and R⁶ are each independently deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q and r are each independently one of integers from 0 to 4,
in Chemical Formula 1-1, R¹ and R² are not simultaneously hydrogen or a methyl group, and
in Chemical Formula 1-2, R¹ and R² are not simultaneously a substituted or unsubstituted C1 to C30 alkyl group.

11. The compound of claim 1, wherein Chemical Formula 1 is represented by Chemical Formula 1-4 or Chemical Formula 1-5: 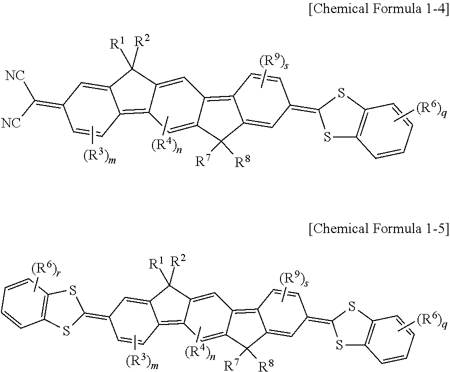
wherein, in Chemical Formula 1-4 and Chemical Formula 1-5,
R¹, R², R⁷, and R⁸ are each independently hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³, R⁴, and R⁹ are each independently deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R⁵ and R⁶ are each independently deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
m and s are each independently one of integers of 0 to 3,
n is one of integers of 0 to 2, and
q and r are each independently one of integers of 0 to 4.

12. The compound of claim 1, wherein Chemical Formula 1 is represented by Chemical Formula 1-6 or Chemical Formula 1-7: 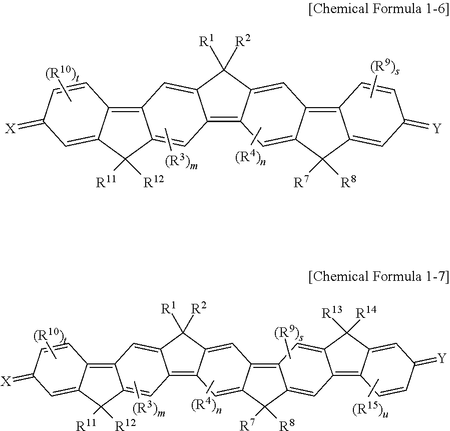
wherein, in Chemical Formula 1-6 and Chemical Formula 1-7,
R¹, R², R⁷, R⁸, R¹¹, R¹², R¹³, and R¹⁴ are each independently hydrogen, deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
R³, R⁴, R⁹, R¹⁰, and R¹⁵ are each independently deuterium, a halogen, a hydroxy group, a cyano group, a nitro group, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, a substituted or unsubstituted C1 to C30 alkoxy group, a substituted or unsubstituted C7 to C30 aryloxy group, or any combination thereof,
X and Y are each independently an electron-acceptor moiety, an electron-donor moiety, or any combination thereof,
in Chemical Formula 1-6, m and n are each independently one of integers of 0 to 2, and s and t are each independently one of integers of 0 to 3, and
Chemical Formula 1-7, m, n, and s are each independently one of integers of 0 to 2, and t and u are each independently one of integers of 0 to 3.

13. The compound of claim 12, wherein
the electron-acceptor moiety is represented by (CN)₂—C═*, wherein * is a portion linked to a position of X in Chemical Formula 1-6 or Chemical Formula 1-7 or a position of Y in Chemical Formula 1-6 or Chemical Formula 1-7, and
the electron-donor moiety is represented by Chemical Formula 2: 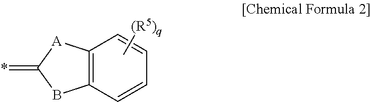
wherein, in Chemical Formula 2,
A and B are each independently oxygen (O), sulfur (S), selenium (Se), or tellurium (Te),
R⁵ is deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C6 to C30 aromatic hydrocarbon group, a substituted or unsubstituted C3 to C30 hetero aromatic hydrocarbon group, or any combination thereof,
q is one of integers of 0 to 4, and
* is a portion linked to the position of X in Chemical Formula 1-6 or Chemical Formula 1-7 or the position of Y in Chemical Formula 1-6 or Chemical Formula 1-7.

14. The compound of claim 1, wherein a molecular weight of the compound ranges from about 250 g/mol to about 1,000 g/mol.

15. The compound of claim 1, wherein a peak absorption wavelength of the compound ranges from about 750 nm to about 3,000 nm.

16. A near-infrared absorbing, near-infrared blocking, or near-infrared absorbing and blocking film comprising the compound of claim 1.

17. A photoelectric device, comprising:
a first electrode and a second electrode facing each other, and
an active layer between the first electrode and the second electrode,
wherein the active layer comprises the compound of claim 1.

18. A photoelectric device, comprising:
a first electrode and a second electrode facing each other,
an active layer between the first electrode and the second electrode, and
at least one charge auxiliary layer between the active layer and the first electrode and/or between the active layer and the second electrode,
wherein the at least one charge auxiliary layer includes the compound of claim 1.

19. An image sensor comprising the photoelectric device of claim 17.

20. An electronic device comprising the photoelectric device of claim 17.
